(19) United States
(12) Patent Application Publication
(10) Pub. No.: US 2017/0319132 A1
(43) Pub. Date: Nov. 9, 2017

(54) DEVICES AND METHODS FOR USE WITH PHYSIOLOGICAL MONITORING GARMENTS

(71) Applicant: L.I.F.E. CORPORATION S.A., Luxembourg (LU)

(72) Inventors: Gianluigi LONGINOTTI-BUITONI, Riverside, CT (US); Andrea ALIVERTI, Como (IT)

(73) Assignee: L.I.F.E. CORPORATION S.A., Luxembourg (LU)

(21) Appl. No.: 15/516,138

(22) PCT Filed: Oct. 1, 2015
(86) PCT No.: PCT/IB2015/002074

§ 371 Date: Mar. 31, 2017

Related U.S. Application Data
(60) Provisional application No. 62/058,519, filed on Oct. 1, 2014.
(60) Provisional application No. 62/080,966, filed on Nov. 17, 2014.
(60) Provisional application No. 62/097,560, filed on Dec. 29, 2014.
(60) Provisional application No. 62/194,731, filed on Jul. 20, 2015.

(51) Int. Cl. A61B 5/00; A61B 5/0205

(52) U.S. Cl. CPC A61B 5/6804; A61B 5/0205; A61B 5/0002; A61B 5/0022

(57) ABSTRACT

A wearable garment including a sensor management system (SMS) network allowing scalable numbers of sensors for communication with a wearable phone. Methods of using the SMS networks are also disclosed.

### CROSS REFERENCE TO RELATED APPLICATIONS

This application claims priority to U.S. provisional patent application No. 62/058,519, filed on Oct. 1, 2014, titled (“DEVICES AND METHODS FOR USE WITH PHYSIOLOGICAL MONITORING GARMENTS”); U.S. provisional patent application No. 62/194,731, filed Jul. 20, 2015 (titled “FLEXIBLE FABRIC RIBBON CONNECTORS FOR GARMENTS WITH SENSORS AND ELECTRONICS”); U.S. provisional patent application No. 62/080,966, filed Nov. 17, 2014 (titled “PHYSIOLOGICAL MONITORING GARMENTS”); and U.S. provisional patent application No. 62/097,560, filed Dec. 29, 2014 (titled “STRETCHABLE, CONDUCTIVE TRACES AND METHODS OF MAKING AND USING SAME”). Each of these applications is herein incorporated by reference in its entirety.

The apparatuses and methods described herein may be related to the following applications: U.S. patent application Ser. No. 14/023,830, filed on Sep. 11, 2013, titled “WEARABLE COMMUNICATION PLATFORM;” U.S. patent application Ser. No. 14/331,185, filed on Jul. 14, 2014, titled “METHODS OF MAKING GARMENTS HAVING STRETCHABLE AND CONDUCTIVE INK;” U.S. patent application Ser. No. 14/331,142, filed on Jul. 14, 2014, titled “COMPRESSION GARMENTS HAVING STRETCHABLE AND CONDUCTIVE INK;” U.S. Provisional Patent Application No. 61/699,440, filed Sep. 11, 2012, titled “SMARTWEAR SYSTEM;” U.S. Provisional Patent Application No. 61/862,936, filed on Aug. 6, 2013, titled “WEARABLE COMMUNICATION PLATFORM;” and U.S. Provisional Patent Application No. 61/950,782, filed on Mar. 10, 2014, titled “PHYSIOLOGICAL MONITORING GARMENTS.” Each of the above applications is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The disclosure herein relates to smart garments, and in particular, electrical connectors for such garments having multiple integrated electrical components (including sensors).

### BACKGROUND

In recent years the development of wearable electronics has dramatically expanded. Computers with ever-faster computer processors enabled faster communication with increased processing speed and improved analysis of vast quantities of data. In addition, sensor technology has also rapidly expanded how patients have been monitored, even by non-professionals. The development of various sensors enabled a variety of measurements to be taken and analyzed by a computer to generate useful information. The use of medical sensing technology in combination with various communications platforms may provide new and interesting ways for people, including patients, to be monitored or to monitor themselves and communicate the results of the monitoring with their physician or caregiver.

Cardiovascular and other health-related problems, including respiratory problems may be detected by monitoring a patient. Monitoring may allow early and effective intervention, and medical assistance may be obtained based on monitored physiological characteristics before a particular health issue becomes fatal. Unfortunately, most currently available cardiovascular and other types of health monitoring systems are cumbersome and inconvenient (e.g., impractical for everyday use) and in particular, are difficult or impractical to use for long-term monitoring, particularly in an unobtrusive manner.

Clothing that includes sensors have been previously suggested. See, e.g., US2007/0178716 to Glaser et al., which describes a “modular microelectronic-system” designed for use with wearable electronics. US2012/0071039 to Debock et al. describes interconnect and termination methodology fore-textiles that include a “conductive layer that includes conductors includes a terminal and a base separately provided from the terminal. The terminal has a mating end and a mounting end.” US2005/0029680 to Jung et al. describes a method and apparatus for the integration of electronics in textiles. These wearable electronic garments are limited however, in their ability to comfortably and accurately link electronics (including sensors) on the garment.

It has been proposed that patient health parameters, including vital signs (such as ECG, respiration, blood oxygenation, heart rate, etc.) could be actively monitoring using one or more wearable monitors, however, to date such monitors have proven difficult to use and relatively inaccurate. Ideally such monitors could be unobtrusively worn by the subject (e.g., as part of a garment, jewelry, or the like). To date, the wearable electronics garments proposed all suffer from a number of deficits, including being uncomfortable, difficult to use and manufacture, and providing inaccurate results. For example, in applications such as US 2012/0136231, a number of individual electrodes are positioned on the garment and connected to a processor by woven conductive fibers or the like; although such garments “require . . . consistent and firm conductive contact with the subject's skin,” in order to provide accurate readings, such designs require that the garment be restrictive in order to prevent movement of the garment (and thus sensors) contacting these skin regions. Such a configuration rapidly becomes uncomfortable, particularly in a garment that would ideally be worn for many hours or even days. In addition, even such tightly worn garments often move relative to the wearer (e.g., slip or ride up). Further, devices/garments such as those described in the prior art are difficult and expensive to manufacture, and are often rather “fragile”, preventing robust usage and washing. Finally, such devices/garments typically do not allow processing of manual user input directly on the garment, but either relay entirely on passive monitoring, or require an interface of some sort (including off-garment interfaces).

The use of garments including one or more sensors that may sense biometric data have not found widespread use. In part, this may be because such garments may be limited in the kinds and versatility of the inputs that they accept, as well as limits in the comfort, and form factor of the garment. For example, sensors, and the leads providing power to and receiving signals from the sensors have not been fully integrated with the garment in a way that allows the garment to be flexible, attractive, practical, and above all, comfortable. For example, most such proposed garments have not been sufficiently stretchable. Finally, such proposed garments are also limited in the kind of data that they can receive, and how they process the received information.

What is needed are apparatuses (including garments) having multiple sensors that may be comfortably worn, yet provide relatively accurate and movement-insensitive measurements over a sustained period of time. It would also be beneficial to provide garments that can be easily and inexpensively manufactured.

In particular, what is needed are stretchable and conductive connectors that can be attached or applied onto a garment. These stretchable, conductive connectors may be used even with the most stretchable of fabrics, and/or with compression fabrics/compression garments, and moved through numerous stretch/relaxation cycles with the underlying fabric without breaking and while maintaining a stable electrical connection over time and use. The apparatuses, including devices and systems including them described herein may address some or all of the problems identified above.

In the last twenty years, the development of mobile telecommunications devices have has dramatically expanded and modified the ways in which people communicate. Computers with ever-faster computer processors enabled faster communication with increased processing speed and improved analysis of vast quantities of data. In addition, sensor technology has also rapidly expanded how patients have been monitored, even by non-professionals. The development of various sensors enabled a variety of measurements to be taken and analyzed by a computer to generate useful information. In recent years, the use of medical sensing technology in combination with various communications platforms has provided new and interesting ways for people, including patients, to be monitored or to monitor themselves and communicate the results of the monitoring with their physician or caregiver. For example, mobile devices such as smart phones have enabled mobile device users to communicate remotely and provided some ability to obtain, analyze, use, and control information and data. For example, a mobile device user may be able to use application software (an “app”) for various individualized tasks, such as recording their medical history in a defined format, playing a game, reading a book, etc. An app may work with a sensor in a mobile device to provide information that a user wants. For example, an app may work with an accelerometer in a smart phone and determine how far someone walked and how many calories were burned during the walk.

The use of a mobile communications platform such as a smartphone with one or more such biometric sensors has been described in various contexts. For example, U.S. Publication No. US2010/0029598 to Roschk et al. describes a “Device for Monitoring Physical Fitness” that is equipped with a heart rate monitor component for detecting heart rate data and an evaluation device for providing fitness information that can be displayed by a display device and is derived by a processing unit, embodied for reading in and including supplementary personal data. U.S. Publication No. US2009/0157327 to Nissila describes an “Electronic Device, Arrangement, and Method of Estimating Fluid Loss” that is equipped with “an electronic device comprising: a processing unit configured to receive skin temperature data generated by a measuring unit, to receive performance data from a measuring unit, and to determine a theoretical fluid loss value on the basis of the received performance data.”

Similarly, clothing that includes sensors have been previously suggested. See, e.g., U.S. Publication No. US2007/0178716 to Glaser et al., which describes a “modular microelectronic-system” designed for use with wearable electronics. U.S. Publication No. US2012/0071039 to Debock et al. describes interconnect and termination methodology fore-textiles that include a “conductive layer that includes conductors includes a terminal and a base separately provided from the terminal. The terminal has a mating end and a mounting end.” U.S. Publication No. US2005/0029680 to Jung et al. describes a method and apparatus for the integration of electronics in textiles.

For example, cardiovascular and other health-related problems, including respiratory problems may be detected by monitoring a patient. Monitoring may allow early and effective intervention, and medical assistance may be obtained based on monitored physiological characteristics before a particular health issue becomes fatal. Unfortunately, most currently available cardiovascular and other types of health monitoring systems are cumbersome and inconvenient (e.g., impractical for everyday use) and in particular, are difficult or impractical to use for long-term monitoring, particularly in an unobtrusive manner.

It has been proposed that patient health parameters, including vital signs (such as ECG, respiration, blood oxygenation, heart rate, etc.) could be actively monitoring using one or more wearable monitors, however, to date such monitors have proven difficult to use and relatively inaccurate. Ideally such monitors could be unobtrusively worn by the subject (e.g., as part of a garment, jewelry, or the like). Although such garments have been proposed, see, e.g., U.S. Publication No. 2012/0136231, these garments suffer from a number of deficits, including being uncomfortable, difficult to use, and providing inaccurate results. For example, in applications such as U.S. Publication No. 2012/0136231, a number of individual electrodes are positioned on the garment and connected to a processor by woven conductive fibers or the like; although such garments “require . . . consistent and firm conductive contact with the subject's skin,” in order to provide accurate readings, such designs require that the garment be restrictive in order to prevent movement of the garment (and thus sensors) contacting these skin regions. Such a configuration rapidly becomes uncomfortable, particularly in a garment that would ideally be worn for many hours or even days. In addition, even such tightly worn garments often move relative to the wearer (e.g., slip or ride up). Further, devices/garments such as those described in the prior art are difficult and expensive to manufacture, and are often rather “fragile”, preventing robust usage and washing. Finally, such devices/garments typically do not allow processing of manual user input directly on the garment, but either relay entirely on passive monitoring, or require an interface of some sort (including off-garment interfaces).

The use of garments including one or more sensors that may sense biometric data have not found widespread use. In part, this may be because such garments may be limited in the kinds and versatility of the inputs that they accept, as well as limits in the comfort, and form factor of the garment. For example, sensors, and the leads providing power to and receiving signals from the sensors have not been fully integrated with the garment in a way that allows the garment to be flexible, attractive, practical, and above all, comfortable. For example, most such proposed garments have not been sufficiently stretchable. Finally, such proposed garments are also limited in the kind of data that they can receive, and how they process the received information.

Thus, existing garments (e.g., devices and wearable sensing apparatuses) and processes for analyzing and communicating the physical and emotional status of an individual may be inaccurate, inadequate, limited in scope, unpleasant, and/or cumbersome.

What is needed are apparatuses (including garments) having one more sensors that may be comfortably worn, yet provide relatively accurate and movement-insensitive measurements over a sustained period of time. It would also be beneficial to provide garments that can be easily and inexpensively manufactured. Finally it may be beneficial to provide garments and apparatuses that are completely safe for use, despite containing electrical components that may be charged or re-charged, including charging from line power.

Described herein are devices, including connectors, that may be used to connect a garment including a one or more sensors and/or a sensor management system (including a microprocessor) to a charger only when the garment is not being worn. Such a feature is important, because it prevents the risk of shock or other hazard to a subject wearing the garment.

### SUMMARY

Described herein are wearable garments (e.g., shirts, vests, harnesses, bras, pants, shorts, scarves, hats, etc.), and particularly garments configured to be worn on a torso, that may include a sensor network distributed over the garment for sensing one or more physiological parameters and/or wearer actions. The vast amount to sensor data that may be generated, often at different data rates, noise levels, formats and locations on the body, may present a dilemma when designing these garments so that they can be both wearable (e.g., comfortable, lightweight, washable, etc.) and accurate (e.g., providing reproducible, medical-sensor grade) outputs. The apparatuses (systems and methods) described herein may provide a solution. In particular, the sensor management system (SMS) networks described herein may address these concerns. These SMS apparatuses may provide distributed network of sensors that may have their (often analog) data locally processed (e.g., at one or more secondary or sub-SMS nodes) before being digitally transmitted to a primary (central, core or overseer) SMS node for further processing and/or aggregation and passing on to a mobile telecommunications device (phone) for presentation, transmission, storage and/or analysis. The phone may connect physically and be mounted to the primary SMS, which may be worn (via the SMS) on the garment in a non-obtrusive location.

For example, described herein are garments that include such apparatuses, such as a garment comprising: a garment body formed of a first fabric; a plurality of sensors permanently affixed to the garment body; a primary sensor management system (SMS) module, wherein the primary SMS module includes: a phone securement connector, a plurality of primary sensor data inputs, a plurality of electrical output connectors, and a housing enclosing processing circuitry coupled to the plurality of primary sensor data inputs and the plurality of electrical output connectors; a plurality of secondary SMS nodes, wherein the secondary SMS nodes each include: a plurality of node sensor inputs receiving input from one or more of the plurality of sensors, one or more node sensor outputs and node processing circuitry comprising a digitizer and an encoder configured to sample analog sensor data from the node sensor inputs and encode a digital representation of the analog sensor data for transmission on the one or more node sensor outputs; and one or more elastic electrical connectors attached to the garment body and connecting the plurality of secondary SMS nodes to the primary SMS module, wherein the one or more elastic electrical connectors each include: an elongate strip of a second fabric, and a plurality of insulated wires extending along a length of one side of the elongate strip in a sinusoidal or zig-zag pattern; wherein the primary SMS module is affixed to the garment body on an outer central back portion of the garment body so that the primary SMS module is positioned on a wearer's upper back when the wearer is wearing the garment.

The housing of the primary SMS module may include a projection region configured to prevent access to a charging port on a phone attached to the phone securement connector.

These apparatuses in particular, may provide the ability to handle a much larger number of sensors (and sensor data inputs) than the number of pins (data inputs) on the primary SMS, allowing the primary SMS to remain small, by distributing the SMS function to sub-nodes that then converge to the primary SMS. The sub-nodes may be very small and low-profile, and may be integrated into the shirt (e.g., as a small chip or circuit board that can be kept soft, thin and non-obtrusive) without offending the fit and comfort for the wearer. For example, the plurality of sensors may comprise a plurality of n sensors and the plurality of sensor data Inputs comprises m sensor data inputs, where m is less than n. The number of sensors (n) may be much greater than the number of data inputs (m) on the SMS, e.g., n may be between 5 and 1000 (e.g. n is between a lower limit of 5, 6, 7, 8, 9, 10, 11, 12, 13, 14, 15, 20, 25, 30 and an upper limit of 10, 20, 30, 40, 50, 60, 70, 80, 90, 100, 150, 200, 250, 300, 350, 400, 500, 600, 700, 800, 900, 1000, where the lower limit is less than the upper limit). The number of sensor data inputs in the primary SMS may be, for example, 2, 3, 4, 5, 6, 7, 8, 9, 10, 11, 12, 13, 14, 15, etc.

As mentioned, the garment body may be any garment, particularly torso-worn garments such as a shirt, a bra, a harness, etc.

Any sensors may be included as part of these apparatuses. For example, the plurality of sensors may include one or more of: stretch sensors, pressure sensors, capacitive sensors, inductive sensors, electrodes, touch point sensors, accelerometers, inertial measurement unit (IMU) sensors, etc. Sensors may be sensors to detect electrical activity, motion, stretch (e.g., respiration), light, temperature, etc. Although some examples of such sensors are provided herein, these should not be limiting. In addition, in some variations the secondary SMS node may be matched or adapted for use with a particular type of sensor. For example, the SMS node may be an accelerometer secondary SMS node, that is configured to process (digitize and transmit) movement/acceleration data. The circuitry associated with the secondary SMS node may be adapted to process the particular sensor data it receives. For example, a secondary SMS node may be adapted to received electrical (e.g., galvanic skin response, ECG, EMG, etc.) data and to filter, amplify, sample (e.g., analog to digital convert, digitize, etc.) the particular data received. Alternatively, a secondary SMS node may be generic to many different types of sensor data and may process it dynamically (or may adjust to the received data). Data from the secondary SMS nodes may be encoded as digital information in any appropriate protocol. In some variations the data is encoded with error correction (e.g., hamming), or optimized for transmission to the primary SMS node, etc. Data may be multiplexed (e.g., data from multiple sensors may be combined (averaged, etc.) or kept separate (time divided) for transmission to the primary SMS. Sensor data transmitted by the secondary SMS node may be encoded with additional information, including labels, such as labels indicating where the data originated, time stamps, quality indicators, etc.

In general, the garments described herein are for use with a mobile telecommunications device (e.g., phone, smartphone, etc.) that may be directly coupled with the primary SMS node. However, in general, the systems and devices described herein may also be used with a processor that is not directly (physically) connected to the primary SMS node, including wireless (Bluetooth, Wifi, etc.) connection to such processors. For example, the primary SMS node may communicate wirelessly directly and wirelessly with a smartphone or other processor. In some variations the primary SMS may itself be a phone or have telecommunications capabilities.

In general, however, the apparatus may include a phone securement connector that is configured to removably secure a phone to the garment. For example, the phone securement connector may include one or more of: a magnetic connector or a mechanical connector.

Any appropriate output connectors may be used. For example, the plurality of electrical output connectors may comprise pin connectors (e.g., pogo pin connectors, etc.).

The processing circuitry of the primary SMS may be configured to serially transfer digitized sensor data received by the primary sensor data inputs to one or more of the electrical output connectors.

Any of the garments described herein may include a phone having a mate for the phone securement connector, to releasably secure the phone to the primary SMS sensor module.

The primary sensor management system may be integrated on an outer portion of the garment body so that the phone securement connector is exposed.

As mentioned, the secondary SMS nodes may be soft and/or low profile. For example, they may not include a housing (e.g., rigid or hard housing) but may be covered in a soft material, such as a foam or epoxy, e.g., covering the processing circuitry and/or connections. For example, all or some (e.g., each) of the plurality of secondary SMS nodes may not be covered by a housing.

In general, the network of primary and secondary SMS nodes may be configured so that the local secondary SMS nodes sample and/or analyze sensor data at a different (typically higher) rate than the transmission rate to/from the primary SMS. For example, the processing circuitry of each secondary SMS node may be configured to sample received analog sensor data at a first rate and to transmit the digital representation of the analog sensor data at a second data rate that is slower than the first data rate.

In any of these variations the flow of information between primary and secondary SMS nodes (and to/from the phone if attached) may be bi-directional. For example, the primary SMS node (independently or via the phone) may transmit control parameters to the secondary SMS nodes. For example, the data sampling rates of the secondary SMS, the data transmission rates (from the secondary to primary SMS), etc. may be controlled by communication from the primary to the secondary SMS nodes. Confirmation of transmission from secondary to primary (or primary to secondary) nodes may also be transmitted and processed. Rules for encoding data may also be transmitted from the primary to secondary SMS nodes. One or both of the primary sensor management system and the secondary SMS node may include (e.g., as part of its circuitry) a memory for storing sensor data, representations of sensor data, or sensor data and representations of sensor data.

In general, the secondary (and in some variations the primary) SMS nodes may be integrated with the connector (e.g., elastic connector, fabric connector, etc.) on the fabric substrate (strip) that is also connected to the fabric forming the garment body. For example, the one or more elastic electrical connectors may be adhesively attached to the garment body. At least some of the plurality of secondary SMS nodes may be affixed to the elongate strip of a second fabric of the one or more electrical connectors.

Each of the insulated wires may be electrically insulated with a thermoremovable insulator. The insulated wires may comprise a bundle of insulated wires that are attached to one side of the elongate strip of second fabric by a stitch at each of a peak and a trough of the sinusoidal or zig-zag pattern, and wherein the length between peak and trough stitches is between about 1 mm and 15 mm. The first fabric and the second fabric may be formed from the same material or from different materials. For example, either of both fabrics may comprise a compression fabric.

Although many or all of the sensors in the garment may be connected to the primary SMS module (e.g., primary SMS node) through connection to a secondary SMS node, in any of these variations one or more sensors may be connected directly (or may also be directly connected) to the primary SMS node. For example, at least one of the sensors of the plurality of sensors may be connected directly to a sensor data input of the primary SMS module.

The primary SMS module housing may include an interface portion, the interface portion including a sensor module interface and a charging interface; wherein the sensor module interface further configured to receive the signals from the plurality of interactive sensors such that the charging interface is fully or partially covered. The charging interface may be configured to receive electrical energy from an electrical energy source configured to engage with the interface portion and charging interface such that the sensor module interface is partially or fully covered. The interface portion, charging interface, and sensor module interface may be configured such that the sensor module interface and charging interface cannot be used simultaneously.

In any of these variations, the primary SMS module may include a battery. Alternatively or additionally, the primary SMS module may receive power from the phone, when connected. The primary SMS may distribute power to the other SMS nodes.

The one or more connectors (e.g., elastic electrical connectors) may have a maximum thickness of less than about 2 mm. The sinusoid or zig-zag pattern of the wires in the connectors may have an amplitude from about 0.5 mm to 15 mm (e.g., from peak to trough). The one or more elastic electrical connectors may have an adhesive coating comprising a hot melt film having a low melting point. The one or more elastic electrical connectors may have an adhesive coating having a thickness of between 10 and 200 micrometers thick.

For example, a garment may include: a garment body formed of a first fabric; a plurality of n sensors permanently affixed to the garment body; a primary sensor management system (SMS) module, wherein the primary SMS module includes: a phone securement connector, a plurality of m primary sensor data inputs, where m is less than n, a plurality of electrical output connectors, and processing circuitry coupled to the plurality of primary sensor data inputs and digitally encoding sensor output for transmission from the plurality of electrical output connectors; a plurality of secondary SMS nodes, wherein the secondary SMS nodes each include: a plurality of node sensor inputs receiving input from one or more of the plurality of sensors, one or more node sensor outputs and node processing circuitry comprising a digitizer and an encoder configured to sample analog sensor data from the node sensor inputs and encode a digital representation of the analog sensor data for transmission on the one or more node sensor outputs; and one or more electrical connectors connecting the plurality of secondary SMS nodes to the primary SMS module, wherein the one or more elastic electrical connectors each include: a plurality of insulated wires extending in a sinusoidal or zig-zag pattern; wherein the primary SMS module is affixed to the garment body on an outer central back portion of the garment body so that the primary SMS module is positioned on a wearer's upper back when the wearer is wearing the garment.

For example, the garments described herein may include: a garment body formed of a first fabric; a plurality of n sensors permanently affixed to the garment body; a primary sensor management system (SMS) module, wherein the primary SMS module includes: a phone securement connector, a plurality of m primary sensor data inputs, where m is less than n, a plurality of electrical output connectors, and processing circuitry coupled to the plurality of primary sensor data inputs and the plurality of electrical output connectors; a plurality of secondary SMS nodes, wherein the secondary SMS nodes each include: a plurality of node sensor inputs receiving input from one or more of the plurality of sensors, one or more node sensor outputs and node processing circuitry connected to the plurality of node sensor inputs and the one or more node sensor outputs; and one or more electrical connectors connecting the plurality of secondary SMS nodes to the primary SMS module.

Further, garments including sensors or other wearable electronics, such as those described herein (including as incorporated by reference) may require close contact between electronics (and sensors) and human body; in such situations it may be important to avoid the possibility of charging the device(s), including components such as phone module batteries, while the device is worn.

Describe herein is one solution, involving the use of four pins that is placed in the same area as the twelve pins used to connect phone module and the female connector that may be placed on the garment (e.g., shirt). These pins represent four phone module USB lines (VBUS, D+, D− and GND) and may be used to both recharge the battery and exchange data (e.g. with a laptop)

For example, in some variations of a wearable electronics system, a charger, a dock, and a USB cable may all be part of the USB charger and part of the final product package. Two variations of schematics of this are shown in FIGS. 2A-2B. In FIG. 2A, the charger has a USB interface in order to be connected with the dock through a USB cable. In this example, the dock includes a port (e.g., a micro USB port) on the side, and an interface (e.g., a four pogo pin interface) on top to be connected with a phone module in a similar way as a female connector does. The dock case (ID) may follow the same basic shape and dimensions of the female connector. For example, FIG. 3A shows an example of the Charge dock ID. FIG. 3B shows an example of an SMS ID that is placed on the garment (e.g., shirt).

In this example, the charger dock and female connector share a very similar ID, which may be useful for safety reasons. To avoid the possibility of charging the phone module battery as long as the device (garment) is worn by users. If users are wearing the garment (e.g., compression shirt or other compression garment), they will not be able to connect the phone module to the charger dock, because phone module and female connector are connected. Similarly, if charger dock and phone module are connected together, users will not be able to connect phone module and female connector. This safety solution may be mandatory for medical certification. As shown in FIG. 4, a phone module and female connector in connected position. In this example, there is no possibility to connect the dock charger.

As mentioned above, also described herein are strips of elastic electrical connectors that may be used to connect multiple electrical devices on a garment having integrated electrical devices (including sensors). These strips of elastic electrical connectors may be adhesively applied to a garment (or a fabric to forma garment) and may be comfortably worn while providing robust electrical connection.

For example, described herein are elastic electrical connector devices for incorporating into a garment to connect multiple electrical components in the garment. Such devices may include: an elongate strip of fabric substrate having a first side and a second side; a plurality of wires extending along a length of the first side of the elongate strip of fabric substrate in a sinusoidal or zig-zag pattern, wherein each of the wires is electrically insulated, and wherein the plurality of wires are attached to the first surface by a stitch at a peak and a trough of the sinusoidal or zig-zag pattern; and an adhesive coating the first side.

As used herein, a sinusoidal pattern is a curve that describes a repeating (or oscillating) pattern, and may broadly include zig-zag, saw-tooth, (e.g., triangular), smooth, or other repeating waves having a peak and a trough, where the peak and trough are connected by non-vertical paths (e.g., excluding purely square waveforms). Thus, in general the oscillating pattern of the wires in any of the apparatuses (e.g., devices, garments, etc.) described herein may be referred to as an oscillating pattern having a series of longitudinally repeating peaks and troughs, wherein each peak is followed by an adjacent trough and separated by a longitudinal distance (e.g., greater than 0.1 mm, 0.5 mm, 1 mm, etc.) and separated by a vertical distance (e.g., amplitude).

Any of these elastic electrical connector device for incorporating into a garment to connect multiple electrical components in the garment may include: an elongate strip of fabric substrate having a first side with a length; a bundle of wires that are twisted together extending along the length of the first side of the elongate strip of fabric substrate in a sinusoidal or zig-zag pattern, wherein each of the wires is electrically insulated with a thermoremovable insulator, and wherein the bundle of wires are attached to the first surface by a stitch at each peak and trough of the sinusoidal or zig-zag pattern wherein the length between peak and trough stitches is between about 1 mm and 15 mm; and an adhesive coating the first side.

The elastic electrical connector may be a generally thin strip (e.g., ribbon, band, etc.) that may be relatively thin and narrow. For example, the strip may have a maximum thickness of less than about 2 mm (e.g., less than about 1.9 mm, less than about 1.8 mm, less than about 1.7 mm, less than about 1.6 mm, less than about 1.5 mm, less than about 1.4 mm, less than about 1.3 mm, less than about 1.2 mm, less than about 1.1 mm, less than about 1.0 mm, etc.).

The elastic electrical connector may be any appropriate length and thickness. For example, the elastic electrical connector (the elongate strip of fabric substrate of the elastic electrical connector) may be between about 0.6 mm and about 3 cm wide, and greater than about 10 cm long. The length may extend for meters, including greater than 1 m, greater than 2 m, greater than 3 m, etc. the elastic electrical connector may be spooled up so that it may be cut to fit and conveniently used in a variety of fabrications.

The plurality of wires comprises a bundle of wires twisted together. In some variations, the plurality may be wires arranged in parallel. The plurality of wires generally includes between 2 and 20 (e.g., between 2 and 18, 2 and 17, 2 and 16, 2 and 15, 2 and 14, 2 and 13, 2 and 12, 2 and 11, 2 and 10, 2 and 9, 2 and 8, 2, etc.). In general, each of the wires is individually coded along its outer length, so that it may be distinguished from the other wires. For example, each wire may be a distinct color and/or pattern (e.g., printed on the outer visible surface of the wire. When the plurality is a bundle of wires, the wires are typically individually electrically insulated. Thus, the bundle is not encased or enclosed as a group, so that they can be individually separated out from the bundle, though pulled out of the stich or attachment holding them to the substrate fabric.

As mentioned, each wire is typically individually electrically insulated, and this electrical insulation may be configured as a thermoremovable insulator that can be removed by application of a relatively low heat, as applied during soldering. Thus, the wires may not need to be separately stripped or removed of the insulation. For example, the wires may be made of a copper wire that is electrically insulated with a polyurethane.

The wires are typically attached on one side of the substrate (fabric) in a sinusoidal pattern, or more specifically a zig-zag pattern. For example, the sinusoid or zig-zag pattern may have an amplitude (from peak to trough, measured in a direction normal to the zig-zag pattern) that is from about 0.2 mm to 20 mm (e.g., from 0.5 mm to about 15 mm, etc.). The distance between the peak and trough measured along the sinusoidal (e.g., zig-zag) pattern, e.g., a length between peak and trough stitches, may be between about 0.5 mm and about 20 mm (e.g., between about 1 mm and 15 mm, etc.).

The elastic electrical connector typically has a relaxed configuration (e.g., unstretched) and a stretched configuration. The garment may be stretched up to about 100% (2x) or more (e.g., 200%, 300%, etc.) of its relaxed configuration without breaking one of the connecting wires.

In some variations, it is helpful that the wires (e.g., bundle of wires) are held to the garment by one or more stitches at the peak and trough of the sinusoidal pattern, as through stitches around the wires that pass through the substrate. This configuration may allow the stitches to act as eyelets that the wires may slide, while still maintaining the shape of the sinusoid.

In any of the elastic electrical connectors described herein the adhesive coating may be a relatively thin adhesive coating. For example, the adhesive coating may comprise a hot melt film having a low melting point. The adhesive coating may have a thickness of between 10 and 200 micrometers thick (e.g., 20 and 190, 30 and 180, 40 and 170, 50 and 160, 60 and 150, etc., or any thickness between 10 and 200 micrometers. The actual thickness may depend on the material, though thinner coatings are preferred. The adhesive is configured to secure the elastic electrical connector to the garment that it will form a part of. Thus, any appropriate garment-compatible (and somewhat elastic and/or flexible) adhesive may be used. For example, the adhesive coating comprises a hot melt film having a melting point of between about 130° C. and 200° C.

In any of these variations, the substrate fabric may be formed of the same fabric as the garment to which the elongate strip of fabric substrate is to be attached, including a stretchable fabric substrate. For example, the elongate strip of fabric substrate may comprise a polyamide/elastane blend fabric (e.g., 74% polyamide, 26% elastane).

Any of these devices (elastic electrical connectors) may include a removable backing on the first side covering the adhesive. The back may be paper (e.g., waxed paper), plastic, or the like, and may be peeled off to expose the adhesive.

Also described herein are elastic electrical connector device for incorporating into a garment to connect multiple electrical components in the garment, the device comprising: an elongate strip of fabric substrate having a first side and a second side; a plurality of wires extending along a length of the first side of the elongate strip of fabric substrate in a sinusoidal or zig-zag pattern, wherein each of the wires is electrically insulated, and wherein the plurality of wires are attached to the first surface; and an adhesive coating the first side.

Method of making these elastic electrical connectors are also described herein. A method of forming an elastic electrical connector that may be applied to a garment to connect multiple electrical components of the garment may include: attaching an elongate bundle of wires to a first surface of an elongate strip of fabric in a sinusoidal or zig-zag pattern comprising alternating peaks and troughs, wherein the wires are each electrically insulated, and wherein the bundle is attached to the first surface by at least one stitch at each peak and trough of the sinusoidal or zig-zag pattern, wherein the length between peak and trough stitches is between about 1 mm and 15 mm; applying an adhesive coating the first side; and covering the adhesive coating with a removable backing.

Also described herein are garments made using the elastic electrical connectors described herein. For example, a garment may include: a first fabric; a plurality of electrical components on the first fabric; and at least one elastic electrical connector comprising: an elongate strip of a second fabric substrate having a first side; a plurality of wires extending along a length of the first side of the elongate strip of fabric substrate in a sinusoidal or zig-zag pattern, wherein each of the wires is electrically insulated, and wherein the plurality of wires are attached to the first surface by a stitch at a peak and a trough of the sinusoidal or zig-zag pattern, and an adhesive coating the first side; wherein the each electrical component is connected to one or more wire in the at least one electrical connector. In general, the electrical components described herein that may be connected by the elastic electrical connectors may include any appropriate electrical component, and in particular (but not limited to) a sensor.

A method of forming a garment may include: adhesively attaching one or more elastic electrical connector to a first fabric, each elastic electrical connector comprising: an elongate strip of a second fabric substrate having a first side; a plurality of wires extending along a length of the first side of the elongate strip of fabric substrate in a sinusoidal or zig-zag pattern, wherein each of the wires is electrically insulated, and wherein the plurality of wires are attached to the first surface by a stitch at a peak and a trough of the sinusoidal or zig-zag pattern, and an adhesive coating the first side; and attaching a plurality of electrical components to the first fabric, wherein each electrical component is connected to at least one wire of the one or more elastic electrical connector.

### BRIEF DESCRIPTION OF THE DRAWINGS

FIG. 1A shows one variation of a garment configured as a shirt, including multiple sensors, which does not include the network of SMS nodes described herein. FIG. 1B is another example of shirt having a plurality of sensors distributed thereon.

FIGS. 2A and 2B show schematics for chargers for a phone module that may be used (e.g., attached to) a garment as described herein.

FIG. 3A shows a schematic of a phone connector dock for docking onto a primary SMS module, shown in FIG. 3B.

FIG. 4 is an example of the outer housing of a phone that may be used with the SMS apparatuses (and garments including them) as described herein.

FIGS. 5A and 5B illustrate connection of a phone to a primary SMS module in a garment, shown as docking onto a back portion of the garment between the user's upper shoulder blades when the garment is worn.

FIG. 6 is a schematic of the connection between a phone (on left) and a primary SMS module (on right).

FIG. 7 is a table illustrating some illustrative specification parameters for a phone that may be used with any of the apparatuses described herein.

FIG. 8 is a table illustrating potential components that may be used with an exemplary SMS network apparatus (e.g., referred to herein as an “X10Y” device or system).

FIG. 9 schematically illustrates one example of a microcontroller that may be part of a SMS node and/or phone as described herein.

FIG. 10 is a table describing some of the terms used in the illustration of FIG. 9.

FIG. 11 is a schematic illustration of a phone that may be used with any of the apparatuses described herein.

FIG. 12 is a listing of various input/output connections (pins) between a phone and a primary SMS module in one example.

FIG. 13 shows the pins described in FIG. 12.

FIG. 14 schematically illustrates another phone example.

FIG. 15 is a back view of another example of the phone of FIG. 14.

FIG. 16 is a sectional view through the phone of FIGS. 14 and 15.

FIGS. 17 and 18 show perspective views of the outer housing (FIG. 17) and inner portion (FIG. 18) of one example of a phone that may be used as described herein.

FIG. 19 is a back view of the phone of FIGS. 17 and 18.

FIG. 20 is a table showing inputs and outputs for the phone of FIGS. 17-19.

FIG. 21 is an example of a pin layout as described herein. FIG. 22 is a table showing the pin names for the arrangement of FIG. 21.

FIG. 23 is a table listing components for one exemplary variation of an SMS node (primary and/or secondary) as described herein. Any of the SMS nodes described may be configured as primary and/or as secondary unless the context indicates otherwise.

FIG. 24 is a schematic illustration of a circuit for a primary SMS.

FIG. 25 is a table listing outputs for an exemplary SMS (primary SMS module).

FIG. 26 illustrates one example of the electrical connector configuration of a primary SMS module as described.

FIG. 27 is an example of a schematic of a primary SMS module circuit board.

FIG. 28A is an outer view of an exemplary housing of a primary SMS module; FIG. 28B is a perspective view of an inside of the housing shown in FIG. 28A.

FIG. 29A is a top schematic view of one variation of a primary SMS module; FIG. 29 B shows a top perspective view of this primary SMS module.

FIG. 30A is a bottom view of view of one variation of a primary SMS module; FIG. 30 B shows a perspective view of the primary SMS module shown in FIG. 30A.

FIG. 31A is a schematic illustration of an elastic electrical connector device for incorporating into a garment to connect multiple electrical components in the garment, shown in a top view.

FIG. 31B is a side view of the electrical connector shown in FIG. 31A.

FIG. 32 illustrates a roll of elastic electrical connector such as the connector shown in FIG. 31A.

FIG. 33 is a schematic illustration of another example of an elastic electrical connector for use in connecting multiple electrical components to a garment.

FIG. 34 shows one example of a bundle of insulated (enameled) wires of an elastic electrical connector connected on one side of a fabric material forming an elastic electrical connector.

FIG. 35 is another example of a bundle of insulated (enameled) wires of an elastic electrical connector connected on one side of a fabric material forming an elastic electrical connector.

FIG. 36 illustrates one example of a secondary SMS node connected to an elastic electrical connector such as those described herein to electrically connect one or more sensors (not shown) with electrical component of the secondary SMS node (e.g., a printed circuit board, or PCB, a UART BUS, multiplexer, encode, sampler/digitizer/A-D converter, clock, memory, etc.).

FIG. 37 illustrates the use an elastic electrical connector such as those described herein to electrically connect with another electrical component (e.g., an external connector).

FIG. 38 illustrates the use an elastic electrical connector such as those described herein to electrically connect with an electrical component (e.g., a strain gauge).

FIG. 39 illustrates the use an elastic electrical connector such as those described herein to electrically connect with an electrical component (e.g., electrodes).

FIGS. 40A-40C illustrate data characterizing the electrical properties and behavior of one example of an elastic electrical connector as described herein. FIG. 40A is a graph showing test results illustrating the voltage through wires of a flexible (fabric) connector having four wires, over repeated cycles of stretching (up to 3000 cycles). FIG. 40B graphically illustrates an example of a connector having six wires. FIG. 40C illustrates an example of a connector having 8 connectors.

FIG. 41 is a schematic section through a bundle of six insulated (enameled) wires that may be used to form an elastic electrical connector.

FIG. 42 is a schematic section through a bundle of four insulated (enameled) wires that may be used to form an elastic electrical connector.

FIG. 43 illustrates on elongate strip of fabric configured as an elastic electrical connector.

FIG. 44 shows an enlarged view of the proximal end of the elastic electrical connector device of FIG. 43, showing the ends of six insulated wires forming the wire bundle arranged in a zig-zag pattern along the length of the elastic electrical connector.

FIG. 45 is an enlarged view of a stretch sensor such as the one shown in FIG. 38, electrically connected to two of the wires of an elastic electrical connector.

FIG. 46 is an example of an elastic electrical connector shown connected to multiple electrical components, including a body ground pad, ECG electrode, and breath sensor. Additional electrical components may also be added.

FIGS. 47A-47F illustrate assembly of an electrode sensor. These figures are showing the new electrode assembly (in this case is the EMG electrode). Take note that this new structure is common to all of our electrodes and basically it is made applying, by thermal process, the ink sensor on a fabric base (not elastic) with glue film (the same of the ribbon) on the other side. This “multilayer” gets holed, then the rivet is inserted in the hole.

FIG. 48 illustrates machining of an electrode sensor by riveting a connector in contact with a conductive ink forming the sensor.

FIGS. 49A and 49B show an EMG electrode. FIG. 49A shows the front (wearer-facing) surface, while FIG. 49B shows the back surface that will be attached to the garment. The sensor may be connected to a connector (e.g., a SPIDON as described herein) and then applied to the garment.

FIG. 50 illustrates soldering of an EMG electrode such as the ones shown in FIGS. 49A and 49B to an electrical connector device by connecting (soldering) at the cap of the attached rivet.

FIG. 51 shows one example of a sensor management system (SMS), shown as a primary SMS module, including a housing, connected to a fabric (e.g., garment), to which an elastic electrical connector may also be connected.

FIG. 52 illustrates an assembled primary SMS module connector and housing attached to a fabric (garment).

FIG. 53 shows a top of a primary SMS module housing.

FIG. 54 shows a bottom of a primary SMS module housing.

FIG. 55 shows another view of a top of a primary SMS housing including an epoxy resin for waterproofing.

FIG. 56 illustrates different housing configurations (e.g., left 20 poles, middle 24 poles, and right 28 poles) for an SMS housing.

FIG. 57A illustrates a multimedia module device (MMM device) mating with an SMS connector, show in partial cross-section.

FIG. 57B shows the MMM device and SMS connector fully mated.

FIG. 58 shows a solder layer of an SMS microcontroller.

FIG. 59 shows a component layer of an SMS microcontroller.

FIG. 60 shows the SMS microcontroller of FIGS. 58 and 59 housed within a primary SMS module housing.

FIG. 61 is another illustration, similar to that shown in FIG. 36, of a secondary SMS node connected to an elastic electrical connector as described herein.

FIG. 62 illustrates another example of electrodes connected to an elastic electrical connector, similar to that shown in FIG. 39.

FIG. 63 illustrates an elastic electrical connector connected to a six-pole female connector and a splitter PCB.

FIG. 64 shows four elastic electrical connectors, each electrically connected to the primary SMS module (connector).

FIG. 65 illustrates an apparatus for use as part of a garment that includes a primary SMS module, a plurality of secondary SMS nodes, and elastic connectors as described herein. This apparatus (referred to herein as a ‘spydon system’) including multiple (e.g., 5) secondary SMS nodes that each contain a plurality of conductive wires connected or connectable distally to multiple different electrical components (e.g., sensors) and connected at a proximal end to the secondary SMS nodes (connectors) and from there to a primary SMS connector in a housing; this entire network may be adhesively and/or otherwise transferred and connected to a fabric to form a wearable garment.

FIGS. 66-69 illustrate an alternative variation of an SMS (FIG. 68) and housing (FIGS. 66, 67 and 69) for the SMS circuitry.

FIGS. 70A-70C illustrate examples of conductive thread sewn into a substrate (e.g., fabric); FIG. 70A shows different patterns of stitches, having different pitches and widths (angles);

FIG. 70B shows an example of five parallel conductive threads that may connect to five different sensors. FIG. 70C shows an example of a single conductive thread (wire).

FIG. 71 illustrates one example of a wired ribbon (an elastic electrical connector) that may be used to connect a stretchable fabric.

FIG. 72 illustrates the attachment of a conductive elastic ribbon formed as shown in above, to a stretch sensor using two wires from the elastic electrical connector.

FIGS. 73, 74 and 75 illustrate one method of making a sealed conductive ribbon (elastic electrical connector) including a stretch sensor coupled to an elastic electrical connector.

FIGS. 76, 77 and 78 show examples of elastic electrical connector that may be adhesively attached to a garment to connect multiple electrical components.

FIG. 79 illustrates one example of a garment including an SMS apparatus as described herein, showing a back view of a garment worn on a torso.

FIG. 80 is an alternative view of the garment of FIG. 79, with another phone connected to the garment via the primary SMS connector.

FIGS. 81A-81C show back, front, and side views, respectively, of one variation of a phone that may be used with the systems described herein.

FIG. 82 is a block schematic of a phone such as the one shown in FIGS. 81A-81C.

FIG. 83 shows a bottom view of a phone such as the one shown in FIG. 82.

FIG. 84 is a circuit diagram of one variation of a control circuit for a phone.

FIG. 85 schematically illustrates I/O ports for a phone that may be used as described herein.

FIG. 86 illustrates one method for controlling power of a phone and SMS apparatus as described herein.

FIG. 87 is an example of a charger that may be used.

FIG. 88 shows exemplary connections on a phone between a phone and a primary SMS module.

FIGS. 89, 90 and 91 illustrates the connections between an exemplary phone and a primary SMS module.

FIGS. 92A-92C illustrate top, front and side views, respectively, of one variation of a primary SMS module.

FIG. 93 is a block diagram of one example of a primary SMS apparatus connected to a phone and a second garment (“tights”).

FIG. 94 is an example of a PCB of an SMS as described.

FIG. 95 is an example of a portion of a primary SMS module.

FIG. 96 is an example of second portion of the housing of a primary SMS module.

FIG. 97 illustrates connection of two portions of an SMS module.

FIG. 98 shows an output connector (pin) layout for one variation of a primary SMS module.

FIG. 99 is a table labeling the pins shown in FIG. 98.

### DETAILED DESCRIPTION

In some example, described herein are phone modules (e.g., PCBA and its plastic external case), from here on referred to as “phone module” (which may be for use with Android, iPhone, or other mobile telecommunications devices), and electronic apparatuses (PCBA and its plastic external case), that may be used with any of the Sensor Management Systems (“SMS”) networks described herein. In general, these SMS networks may be used as part of a garment and may include: a primary SMS module, a plurality of secondary SMS nodes distributed on the garment and receiving input from a plurality of sensors on the garment, and flexible (e.g., elastic) connectors) connecting the network of secondary SMS nodes to the primary SMS nodes; the phone may connect to the primary SMS module.

During a normal usage, a phone module and an SMS may stay connected and work together. The SMS may be attached to a piece of fabric, from here on referred as a garment or fabric band (for simplicity). The fabric band typically embeds a series of biometric sensors (e.g. heart rate sensor) that will be managed and controlled by the SMS. Examples of the manner in which the SMS may be attached to the fabric band and exemplary specifications for the sensors may be found, for example in U.S. patent application Ser. No. 14/023,830, titled “WEARABLE COMMUNICATION PLATFORM;” filed on Sep. 11, 2013; U.S. patent application Ser. No. 14/331,185, filed on Jul. 14, 2014, titled “METHODS OF MAKING GARMENTS HAVING STRETCHABLE AND CONDUCTIVE INK;” U.S. patent application Ser. No. 14/331,142, filed on Jul. 14, 2014, titled “COMPRESSION GARMENTS HAVING STRETCHABLE AND CONDUCTIVE INK.”

In general, a fabric band may be worn by users, putting the sensors in direct contact with the users' skin and the SMS in close contact with their backs, just under the neck (FIGS. 5A and 5B). FIG. 5A shows the phone module (B) positioning in respect to the SMS (A) and user's back.

In this example, eight neodymium magnets embedded into the SMS case as well as in the phone module case will magnetically couple these two parts; moreover the phone module will be inserted into a pocket, for extra stability (FIG. 5B), showing a phone module positioning on the fabric band; in this example, a pocket may improve the stability.

In addition to this magnetic/mechanical coupling, twelve pins on each part (female pins on the SMS and male pins on the phone module) may also guarantee an electrical connection between them. Specifications for the electrical connections will be given below. The phone module may actually have sixteen pins and not twelve as the SMS; the additional four pins may be used as a custom USB port. Again, more details about this custom USB port will be given into the next sections.

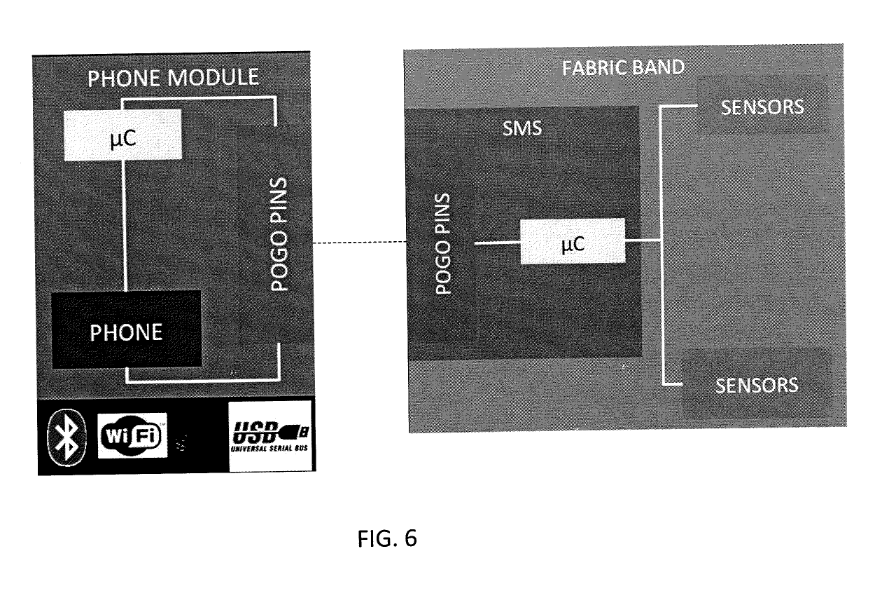

A block diagram of the whole wearable device is shown in FIG. 6. The dashed line represents a magnetic (and electric) coupling between the two parts.

A customized phone module may be used, or a generic one. For example, a phone module may include the technical specifications listed below. A phone module could be a standard smartphone, or could be modified to operate without touch screen display and other components, such as cameras and external speakers. In some variations the phone module may include an additional custom electronic part to interact with SMS and sensors. As shown, a phone module may be connected to an SMS, which is placed on the user's back. During normal usage, the phone module will stay attached onto the user's back. The technical specifications and requirements discussed in this section are divided into the following sub-sections: 1. General phone module specifications; 2. Specifications for the additional electronic part to be added on the phone module PCBA, from here on referred as “X10Y electronics”; 3. Requirements for the elements positioning on the phone module PCBA; 4. Software and Firmware requirements 5. Specification for the phone module case; 6. Other requirements.

### General Phone Module Specifications

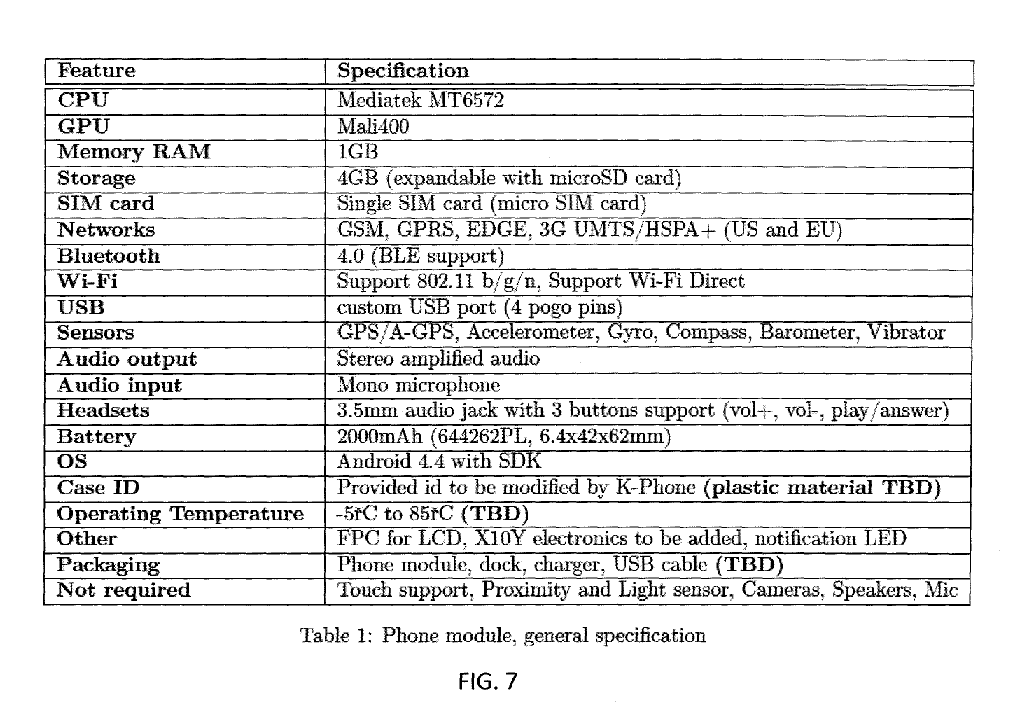

Since a phone module could be a standard smartphone, it may include most of the main specifications typical of any other smartphone on the marker, however, some components will not be needed. An exemplary list of the included phone module features given below and Table 1 (FIG. 7) provides a less detailed list of those features in one example. Not that necessary (required)/not necessary features are specific to this example and may be included or excluded in other examples.

CHIPSET. Mediatek MT6572 is considered a good compromise between performances and final cost.

MEMORY. The internal memory (RAM) should be IGB, while the internal storage may be 4 GB, expandable with external microSD cards (T-Cards).

SIM CARD. A single slot for microSIM card is useful.

NETWORKS. Despite LTE support is not required, 3G networks may be supported. In addition, it is helpful to have a wide network compatibility. Simultaneous support for networks of different global areas (US and EU) is required. A single version for the US and EU markets is useful.

BLUETOOTH. Bluetooth 4.0 is required, since it is useful to have a Bluetooth Low Energy support.

WI-FI. Wi-Fi antenna must support 802.11 b/g/n (1×1). Wi-Fi Direct support may also be also included.

USB. A custom USB connector may be placed inside the phone module cavity (see phone module id as reference). Four male pogo pins will be used as USB port. The correspondent four female pins will be placed on a dock, part of the phone module charger (see the charger sections as reference).

SENSORS. The sensors may be: GPS (A-GPS support), accelerometer, gyro, compass, barometer, and vibrator.

AUDIO INPUT. The audio input line is mono, and may be amplified.

AUDIO OUTPUT. The audio output lines may be stereo, and may be amplified.

HEADSETS. A 3.5 mm audio jack should to be places in the upper part of the PCBA (see following sections as reference). The phone module may support a three buttons headset (volume+, volume−, play/answer).

BATTERY. The battery capacity may be 2000 mAh. The selected dimensions are, for example: 6.4×42×62 mm (644262PL).

OS. The phone module may have Android 4.4 or greater. The Android OS may have the following characteristics: root, English language, no lock screen, and open Bluetooth (no user confirmation for Bluetooth pairing requests).

CASE ID. The phone module case may be made of plastic. Different plastic materials may be taken into account as it will be discussed into the “external case specifications” section. (e.g. add USB pins, add audio jack, add access to SIMcard slot).

OTHER. FPC for connecting LCD screen may be added on the phone module PCBA.

This feature may be used during testing phase and not be available for final users. An additional electronic part (X10Y electronics) may be added on the phone module PCBA (see following sections as reference). Lastly, support for a multicolor notification LED may be included.

NOT REQUIRED. Cameras (front and back), proximity sensor, ambient light sensor, external speakers and microphone are not required.

### X10Y Electronics

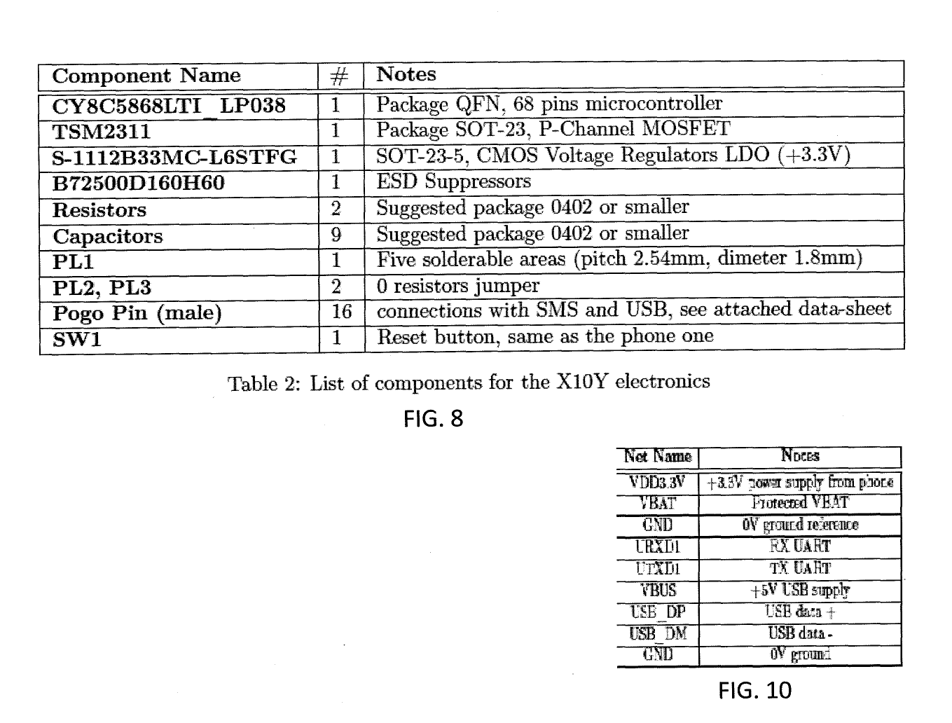

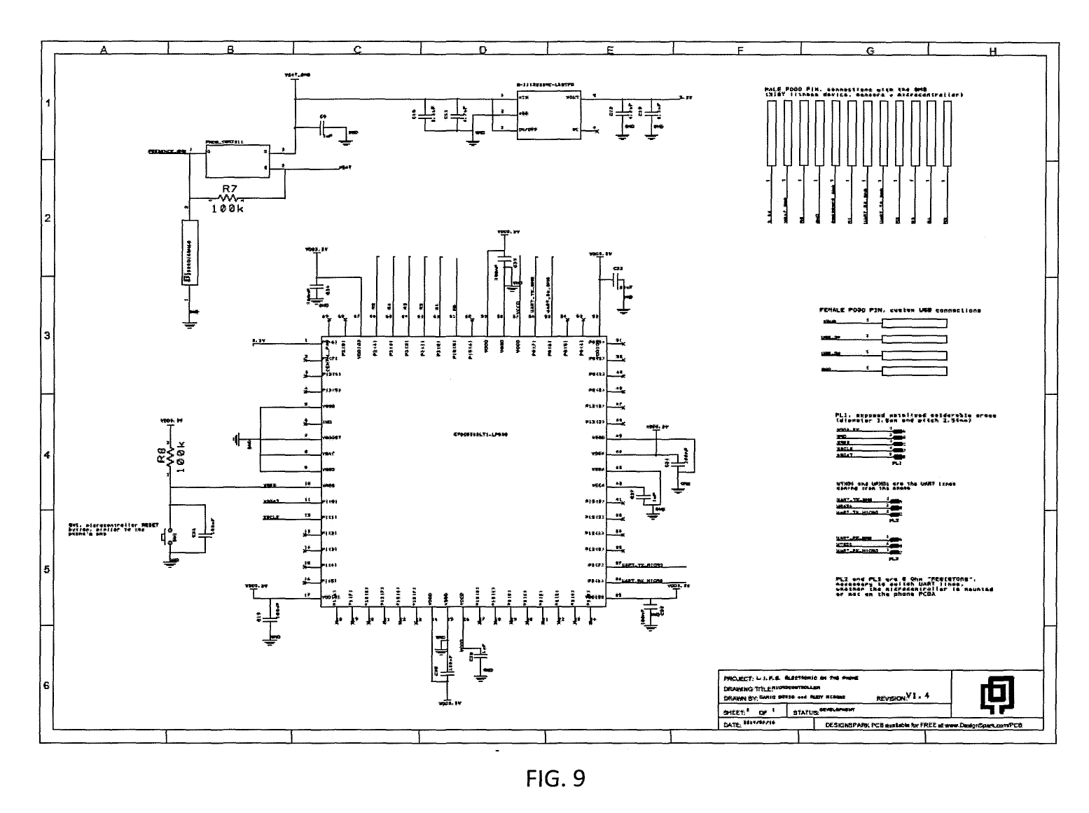

X10Y electronics is an additional electronic part that may be placed on the phone PCBA. The phone module PCBA may be then given by the combination of two electronic parts: X10Y electronics and Android phone electronics. In this example, eleven different electronic components define the X10Y electronics schematic. Table 2 (FIG. 8) shows a list of these components and their amounts. FIG. 9 shows an example of an X10Y electronics schematic. The two electronic parts (X10Y electronics and phone electronics) may be connected by nine electrical lines, or nets (Table 3, FIG. 10). These nets ensure a digital communication, a supply for the SMS (phone battery supplies X10Y electronics and SMS), and a USB line for data exchange (e.g. with a laptop) and battery charging. More details about the USB connector and charger will be given into the next sections.

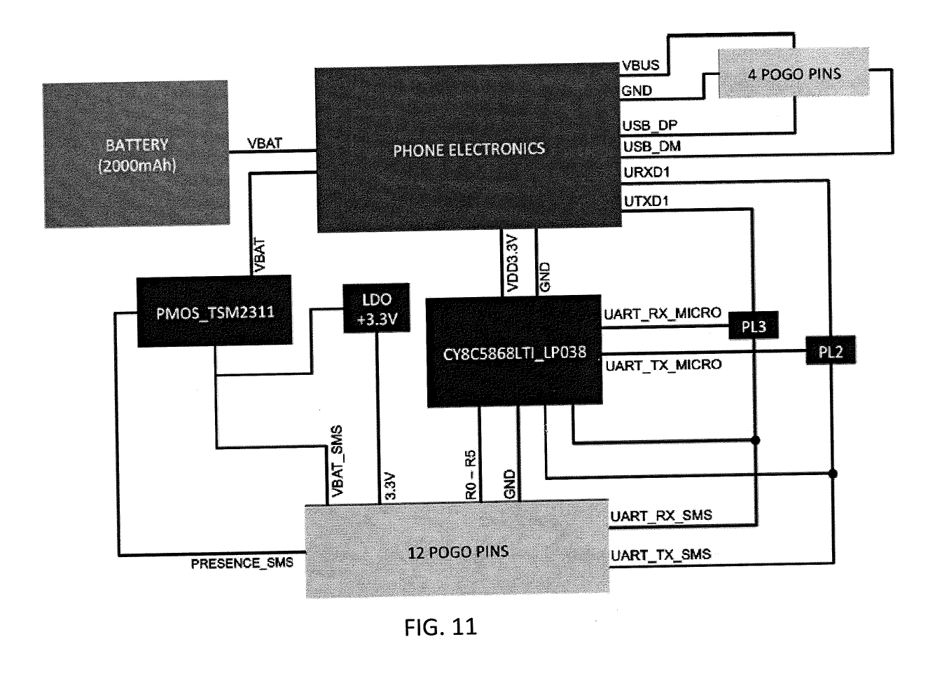

The digital communication line may be based on a serial TTL UART, with a baud rate of 921600 and a logic voltage level of +3.3V. FIG. 11 shows the block diagram of the connections between X10Y electronics and phone electronics. FIG. 11 is a block diagram of connections between X10Y electronics and phone module electronics.

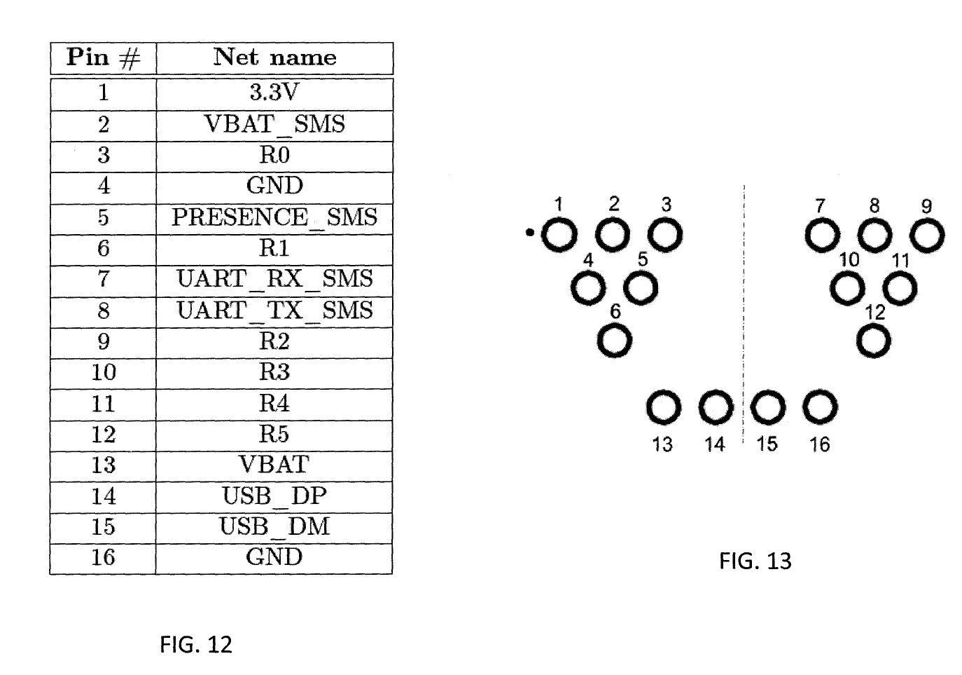

The pinout for the sixteen pogo pins placed on the phone module PCBA is shown in Table 4 (FIG. 12). Table 4 shows one example of phone module pogo pins pinout. FIG. 13 shows the pins numbering seen from the bottom of the phone module id: pin number 1 is always marked by a dot and should be taken as reference from here on. Net names used are referred to as in FIG. 9. FIG. 13 shows Pogo pins numbering for the phone module PCBA, view from the bottom.

### PCBA Dimensions and Elements Position

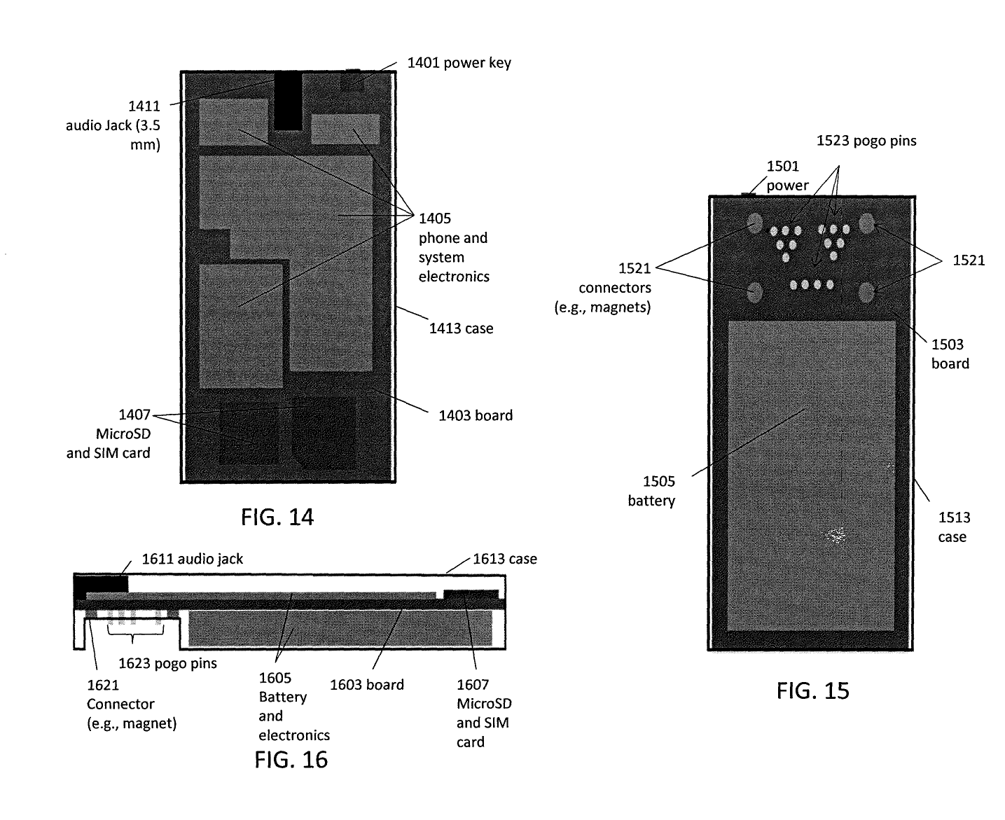

The position of each element on the phone module PCBA may vary, and the phone ID may be kept as small as possible. The following figures: FIGS. 14-16, respectively show top, bottom and lateral-section views of one variation of a phone module PCBA and relative elements placed in the exemplary positions. Shape and dimensions of these figures may not be representative. For example, FIG. 14 shows a top view of the phone module PCBA. FIG. 15 shows a bottom view of the phone module PCBA. FIG. 16 is a lateral-section view of the phone module PCBA.

FIG. 14 shows the top view of the phone module PCBA (in green) and external case (black line). In red, the power button. In black, the 3.5 mm audio jack. In grey the phone module electronics. In blue, microSIM card and microSD slots. Lastly, in purple, the microUSB connector. FIG. 15 shows the bottom view of the phone module PCBA (in green) and external case (black line). In orange four neodymium magnets (see the attached data-sheet). In yellow, sixteen male pogo pins. Lastly, in gold, the phone battery. FIG. 16 shows the lateral-section view of the phone module PCBA (in green) and external case (black line). The same color scheme as before was also used here.

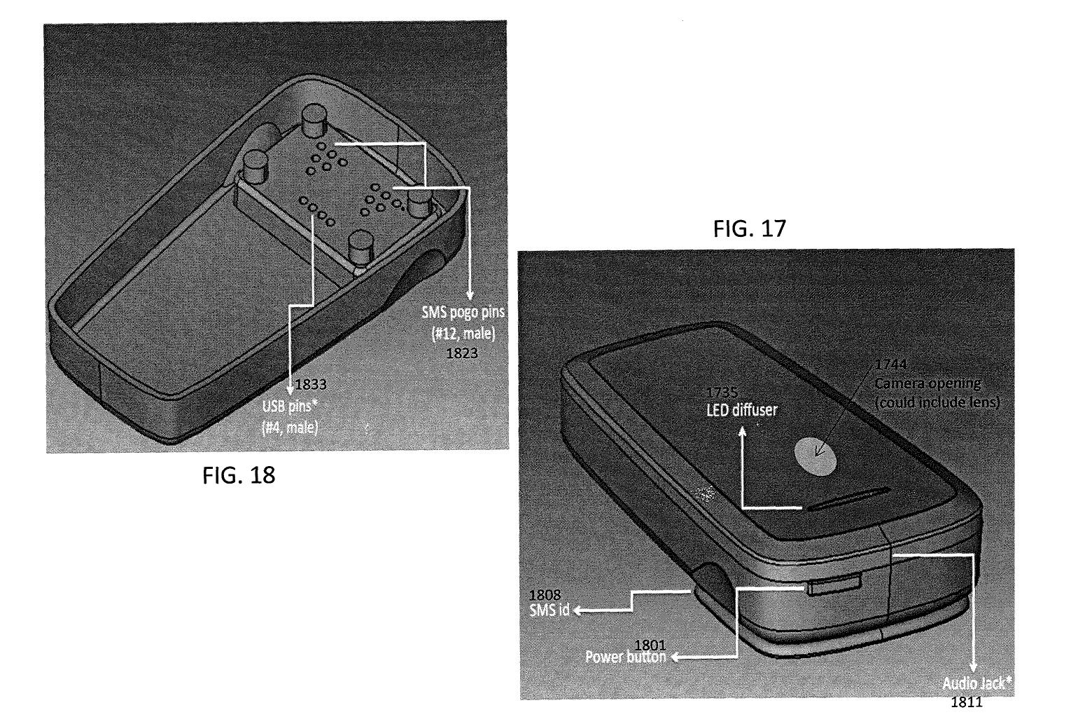

The main functions of the phone module USB port are: charging the battery and exchanging data. Thus, thus device may be configured to prevent a user from charging the battery while the device is being worn. For example a custom USB connector may be included inside the phone module cavity, where the pogo pins are placed. In this way, the user will be prevented from changing the battery as long as the device is connected to the SMS (and the garment). This is shown and described in greater detail below with reference to FIGS. 17-19.

### Software and Firmware Specification

A simplified Android ROM may be used to improve the final user experience and phone module performances. During mass production, this customized ROM may be loaded on every phone module. Phone modules may have the following characteristics: root, English language, no lockscreen, and open Bluetooth (no user confirmation for Bluetooth pairing requests).

### External Case Specification: Case Id and Md

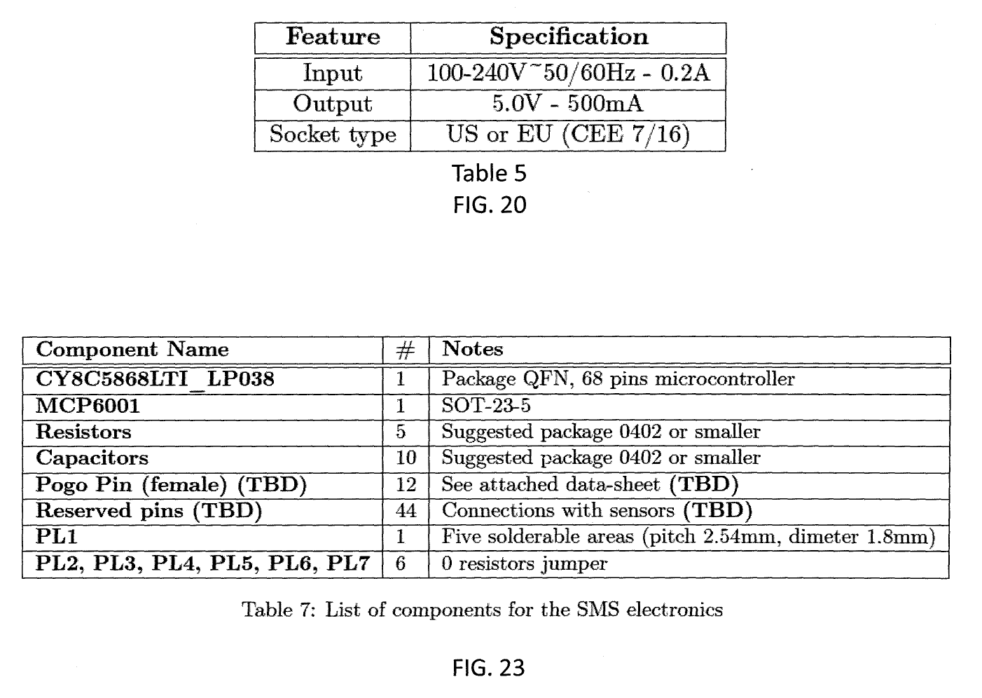

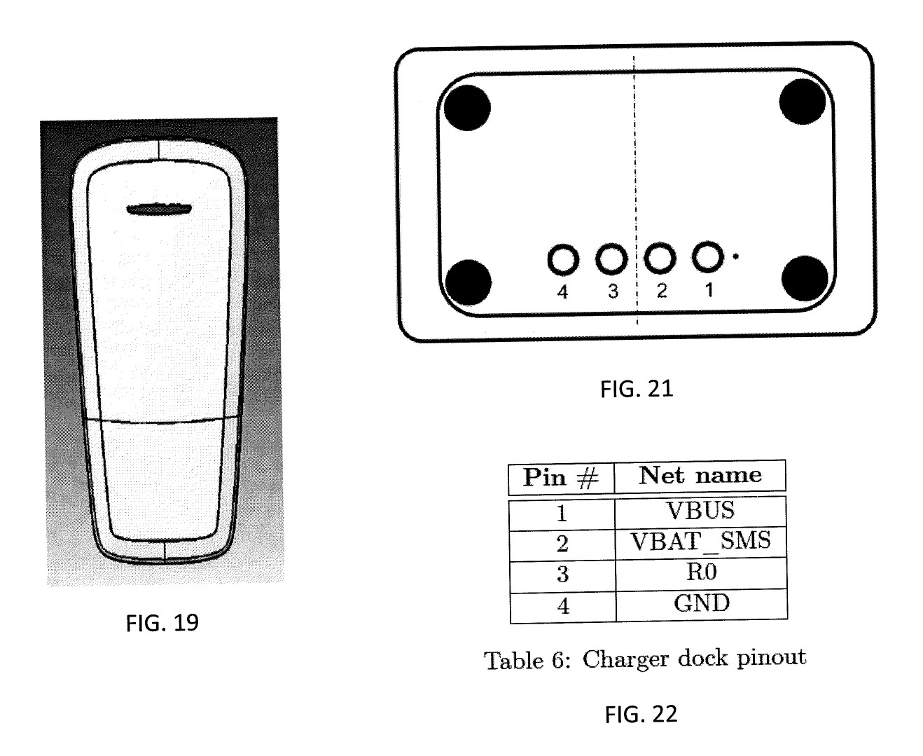

An example of a phone module case id is shown in FIG. 17. The phone module id involves two main parts: a lower shell (FIG. 18) and an upper shell. The lower shell may be attached and locked to the upper one by screws. This may avoid any unnecessary access to the internal PCBA and battery. On the contrary, access to microSIM card and microSD (T-Card) slots may be guaranteed, as well as the access to audio jack. The SIMcard slot (and microSD) access may be similar to that shown in FIG. 19. The holes for the twelve pogo pins to communicate with the SMS are visible in FIG. 18. The holes for the four additional pins (custom USB port) are also indicated. The position of these four pogo pins may match the position of the pins placed on the charger dock (see charger section as reference). The provided id also hosts the housings for four neodymium magnets in the lower shell (FIG. 18i).

An LED diffuser may also be included, e.g., placed on the upper shell. This diffused may guarantee the view of the notification LED light. The external case may be made of plastic. Materials e.g., (plastic coated by rubber material) can give a nice soft touch while keeping flexibility and stiffness. In addition, considering that they do not have a glossy surface, they could improve the grip and stability between phone module case and SMS.

### Power Key

Considering the phone module architecture, the power key may be the primary (or only) way users will be able to directly interact with the phone. Its functionality assumes a key role for the user's experience. Here following a series of functionalities related to the use of the power key:

Power ON. The phone should be turned on by long pressing the power key for 3 seconds while the phone is turned off. If the turning on action is correctly performed, the notification LED will be turned on for a fixed amount of time (e.g. 3 seconds).

Power OFF. The phone should be turned off by long pressing the power key for 3 seconds while the phone is turned on. If the turning off action is correctly performed, the notification LED will be turned on for a fixed amount of time (e.g. 3 seconds). At the Android OS level, it may be important to avoid any sort of confirmation dialog for switching off the phone. The phone must be turned straight off after pressing the power key for 3 seconds without any other user interaction.

Check battery charge. The battery charge should be checked by a short press of the power key both with the phone turned on and off. The user should be then always able to check the current state of the battery charge.

### Notification LED

The notification LED may be a multicolor LED used to interact with users by giving simple information. Here following a series of functionalities related to the notification LED:

Power ON. As said the notification LED should be turned on for a fixed amount of time (e.g. 3 seconds) if the phone is correctly turned on. In this case the LED light is green.

Power OFF. As said the notification LED should be turned on for a fixed amount of time (e.g. 3 seconds) if the phone is correctly turned off. In this case the LED light is red.

Check battery charge. By single pressing the power key the user can check the current battery charge. The battery may assume three states: charged (between 100% and 45%), half charged (between 45% and 15%) and discharged (between 15% and 0%). Depending and the state the notification LED will assume different a color: green for charged state, yellow for half charge state, and red for discharged state. Considering that the user may not be aware of the current battery state if the charge runs under a pre-fixed level of 5% the notification LED should be persistently blink in red with a frequency of 0.2 Hz.

Other notifications. Other notifications could be taken into account while developing our custom Android ROM. L.I.F.E. will need to have access to the notification LED.

The phone module will have all the connectivity features (2G/3G, Bluetooth, WiFi, GPS) typical of any other Android smartphone on the market. Of course, those connectivity features will involve the use of antennas. The phone module may be placed and left on the user's back, as was shown above.

The phone module may be kept in close contact with the human body, thus it may be important to configure the devices so that normal phone functionality does not create unexpected effects (e.g. overheating).

### Phone Module Charger (TBD)

Considering the close contact between electronics (and sensors) and human body it may be important to avoid the possibility of charging the phone module battery as long as the device is worn by users. The solution described herein involves the placing of four addition pogo pins in the same area as the twelve pogo pins used to connect phone module and SMS. Those pins should be connected to the four phone module USB lines (VBUS, D+, D− and GND) and will be used to both recharge the battery and exchange data (e.g. with a laptop).

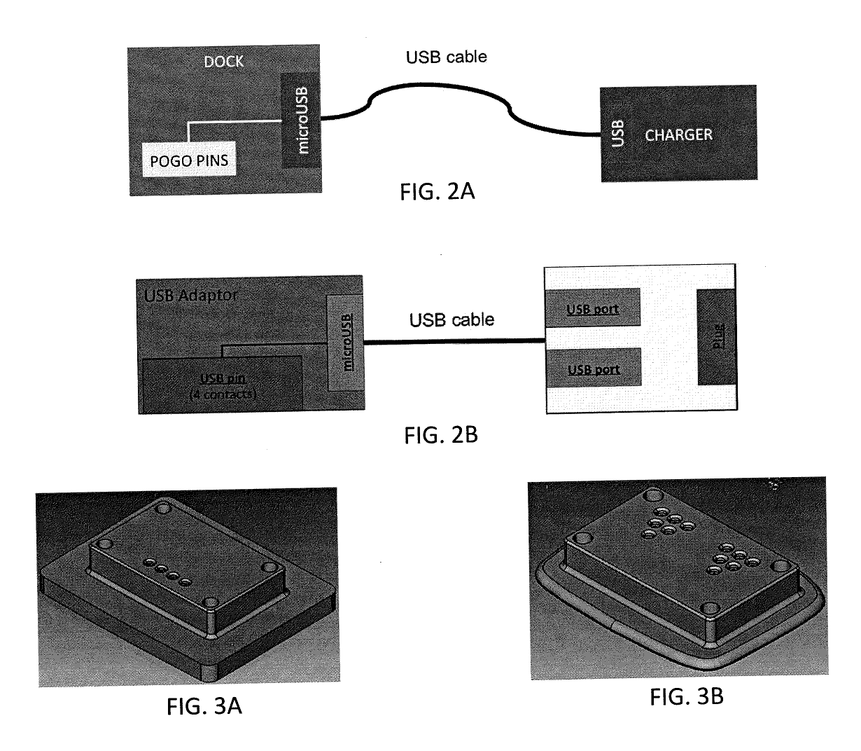

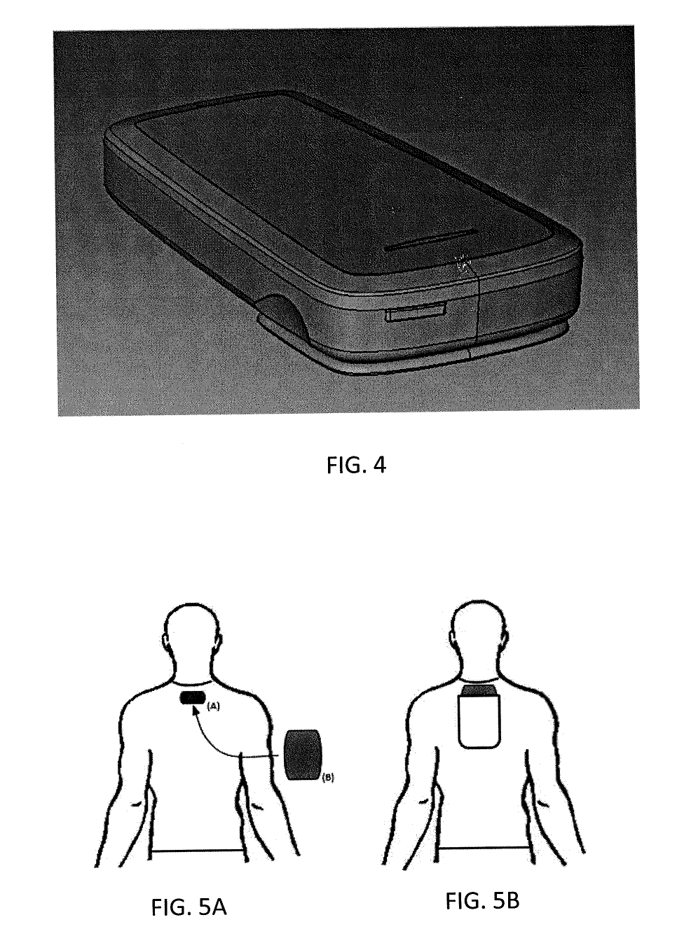

A charger, a dock, and a USB cable will be part of the phone module charger and will be all part of the final product package, as illustrated above in FIGS. 2A-2B. The charger may have a USB interface in order to be connected with the dock through a USB cable. The charger socket may be changed depending on the target market. For example: US and EU (CEE 7/16). As reference, some of the possible specifications for the charger are shown in Table 5 (FIG. 20).

A dock may have a microUSB port on the side, and a four pogo pins interface on top necessary to connect with the phone module in a similar way as the SMS will do. The dock id may be developed by following the same basic shape and dimensions of the SMS id. It is clear that the fact that charger dock and SMS share a similar id has a specific reason: for safety reasons it is extremely important to avoid the possibility of charging the phone module battery as long as the device is worn by users. If the user is wearing the compression garment (e.g., fabric band) and phone module and SMS are connected, she/he will not be able to connect the phone module to the charger dock. Similarly, if charger dock and phone module are connected together, the user will not be able to connect phone module and SMS.

FIG. 3A shows an example of a dock id. It is clear that this part shares a similar shape with the SMS id (see next section), despite some modifications are required: four female pogo pins (instead of twelve) and a higher height of the base support. It should be noted that the four pins placed here will have to match the four pogo pins on the phone module id (used as custom USB port). The system may not require any sort of electronic component inside the dock, the USB lines coming from the four pins may be connected to the microUSB port following the requirements shown in FIG. 21 and Table 6 (FIG. 22). It should be noted that once again some magnets may be embedded into the dock id in order to keep it attached to the phone module.

### SMS Network

An SMS network generally includes a primary SMS module or device (or portion of a device) able to acquire data from a series of sensor placed on the device (e.g., compression fabric) described above. An SMS may be placed on the fabric and may communicate with the phone module through a serial UART line.

As already seen for the phone module, the primary SMS module may include any connector (e.g., four neodymium magnets) in order to guarantee a (e.g., magnetic) coupling between the two parts. Twelve pins (female pogo pins) will then allow the two part to be electrically connected.

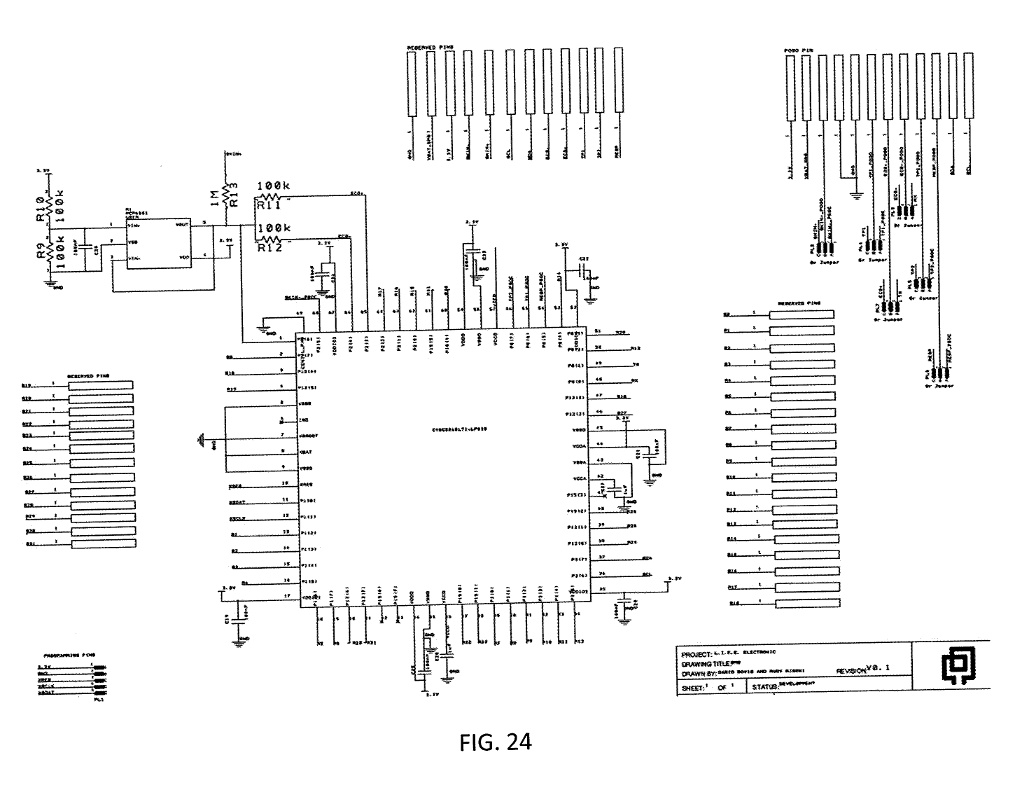

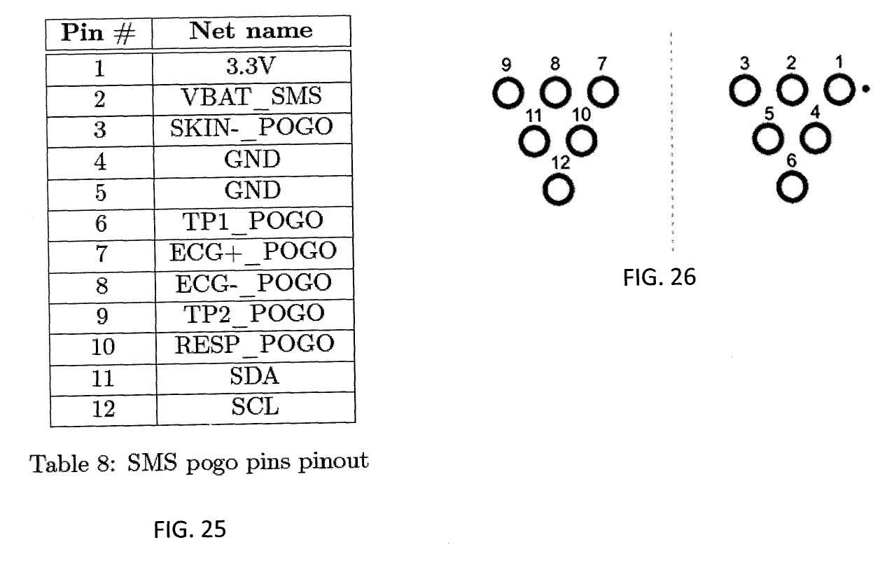

Eight different electronic components may be included as part of an SMS schematic. Table 7 (FIG. 23) shows a list of these components and their numerosity, while FIG. 24 shows an example of an SMS schematic. The pinout for the twelve pogo pins placed on the SMS PCBA is shown in Table 8 (FIG. 25). FIG. 26 shows the pins numbering from a top view: pin number 1 is always marked by a dot and should be taken as reference from here on. Net names used should be referred to FIG. 24.

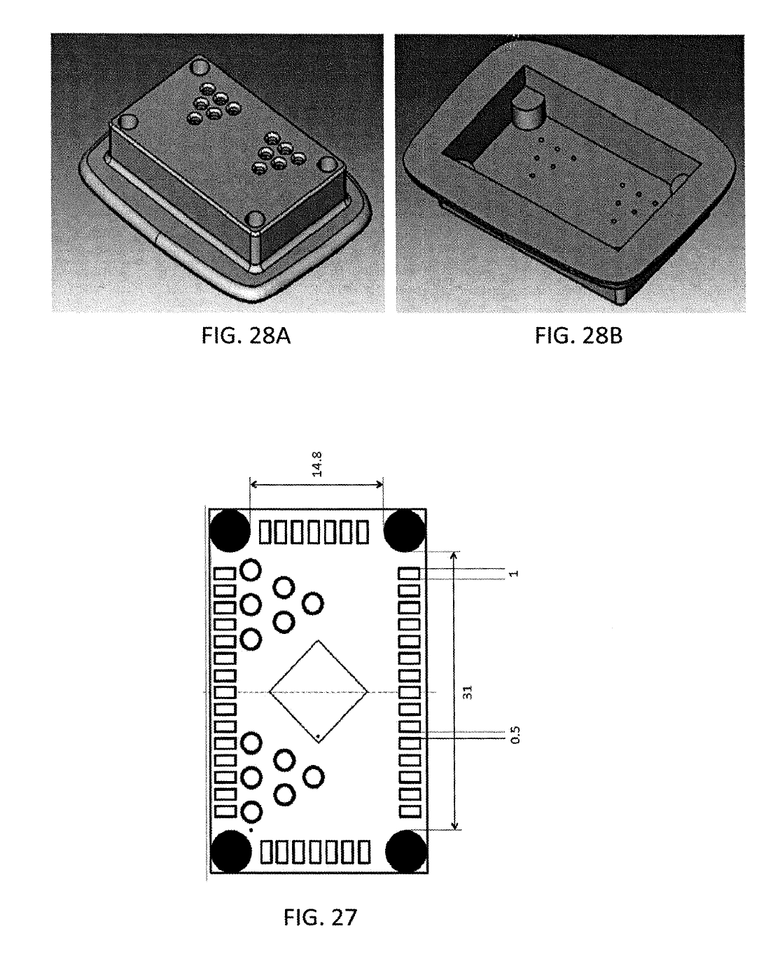

The layout of the SMS PCBA may follow a series of specifications and requirements. As reference, an example of the required position for microcontroller and solderable areas onto the PCBA is shown in FIG. 27. Exemplary dimensions of the PCBA are shown in FIG. 27.

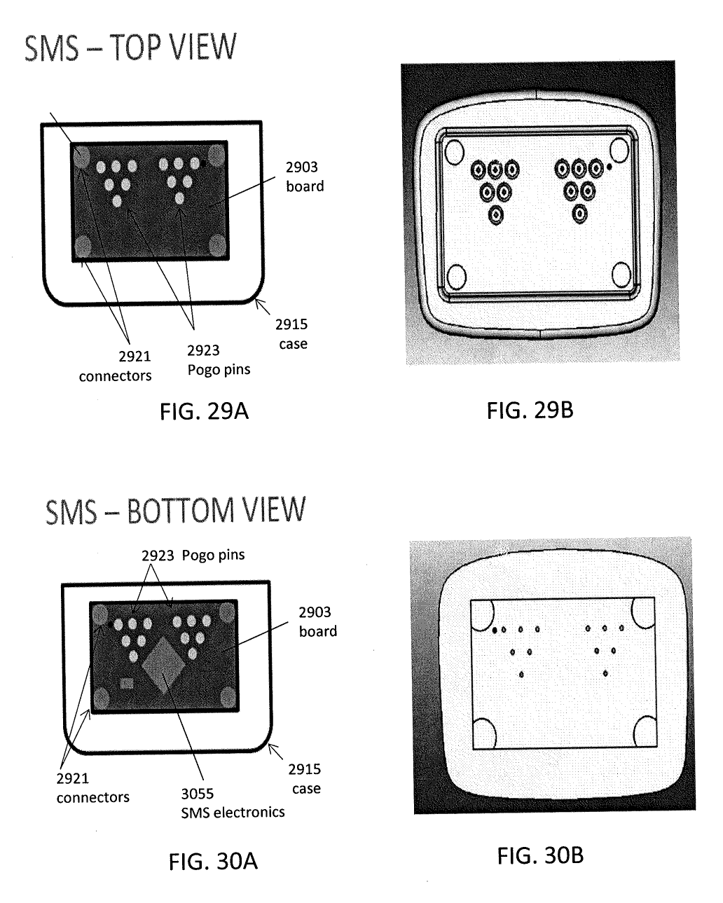

An exemplary SMS case id is shown in FIGS. 28A and 28B. Holes for twelve pogo pins and housings for four neodymium magnets are well visible in FIG. 29A. Considering that this part will be place in close contact with the human body, the material choice plays a crucial role for the user experience. The external SMS case is intended to be made of plastic, and/or may be conditioned, altered or treated to give a better “touch experience” and grip with the phone module case.

FIGS. 29A-30B illustrate top and bottom views, respectively of an exemplary SMS. In this example, the SMS PCBA may be placed in direct contact with the SMS case, and thus all the electronic components may be placed on the PCB bottom layer. The SMS pogo pins may go through the PCBA. Pin number 1 is shown marked with a dot.

In general, a Manager System (SMS) may be placed directly onto the garment (e.g., shirt, shorts or in any other component of the wearable device, i.e. balaclava, socks, gloves, etc.). The SMS may include an electronic board. Connections to the SMS may be made by semi-rigid materials (e.g., Kapton) that may be included as part of the garment.

An SMS that is integrated into the garment (as opposed to being provided by a separate device such as a smartphone) may provide numerous advantages. For example, an integrated SMS can manage a larger number of connections with the different sensors, and may processes the signals and communicates with the phone by means of a single mini-USB cable (e.g., independently of the number of signals processed). No matter the number of sensors that will be included in future devices (e.g., shirt, thighs, gloves, socks, balaclava, etc.), the connection between SMS and sensor module (e.g., phone) may always be based on a single 5-pin USB connection, thus substantially reduce the size of the female and male connectors from the device to the phone module. In a typical configuration, an SMS connects to a male connector through a UART (Universal Asynchronous Receiver-Transmitter) module and the male connector communicates to the mobile through another UART and an UART-to-USB module (see attached schematic and drawings).

An integrated SMS can be placed in different locations on the garment. For example, it may be placed at the base of the neck between shoulder blades, on the lumbar region on the thighs or even on the socks, gloves, balaclava, etc.

An SMS may also be configured to communicate with different phones for the device.

As mentioned, an integrated SMS may also allow you to have more connections (pins) to connect to different sensors/outputs. For example, an accelerometer may need 5 pins if you have the SMS present in a sensor module (e.g., mobile phone); an SMS integrated into the shirt may need fewer connectors, for example, such an SMS may need only 2 pins. With more sensors, without an integrated SMS the number of connectors may become unfeasible.

In general, the SMS may be a module (chip) that manages the signals from and to the sensors, and may act as an interface between the communication system (sensor module configured from a phone, etc.) and sensors. The SMS may manage the connection and interfaces between them. For example, and integrated SMS may include physical connections to sensors and may manage the way in which the signals are processed and sent between sensors and a sensor module and/or other analysis or control components. The SMS may also include or may connect to a multiplexer to alternate readings between various sensors to which it is connected.

In some variations, a SMS may provide proper power supply to passive sensors or active sensors. An SMS may take power from the mobile systems through a port such as a USB port. An integrated SMS may communicate from one side to a sensor module (e.g., communications systems/phone, etc. configured as a sensor module) through a USB port. The SMS may act as an interface or a bridge between the sensors and the sensor module.

In addition, any of the integrated SMSs described may be configured to include on-board processing (e.g., preprocessing), including, but not limited to: amplification, filtering, sampling (control of the sampling rate), and the like; typically basic pre-processing. An integrated SMS may also encode signals from the one or more sensors. In some variations the SMS may include a microcontroller on board. Further, and integrated SMS may also generally manage communication protocols to/from any or all of the sensors, and may make an analog to digital conversion (if the signals are analog) and may also communicate with a comm port of a USB, before going to the USB. For example, an SMS may be configured to convert the signal into UART to the USB signal protocol.

In addition or alternatively, any of the integrated SMSs may be configured as a signal receiver/transmitter. For example, an SMS integrated into the garment may be adapted to convert parallel signals to serial signals (in the order of the data).

As mentioned, an integrated SMS may be placed in any position on a garment, e.g., on or near the neck region, or more peripherally. Although the SMSs describe herein are referred to as “integrated” SMSs, these SMSs may be included on or in the garment (e.g., in a pocket or enclosure, though in some variations it is not physically connected/coupled to the fabric, but is instead placed on the garment. Thus, any of these SMSs may instead be referred to as dedicated or specific SMSs rather than (or in addition to) integrated SMSs. For example, the SMS may be placed under the female connector (housed inside the female connector), as part of the garment. When you wash the garment the SMS may get washed with the connector and the chip; the pins and SMS are waterproofed.

In some variations, the connectors (e.g., pins/ports) of the SMS are adapted to water resistant/water proof. For example, the pins used may make connections that are waterproof, e.g., with connections that only open when you engage the male pin, but are otherwise closed and waterproofed.

In any of these integrated SMSs, the SMS is a part of the garment, and are worn with the garment; the SMS module may pre-process the signal(s) to prepare them for transfer.

Thus, in any of the garments described, an SMS (Sensor Management System) may be included that is positioned on each garment (onboard/dedicated), rather than separate from the garment, e.g., as part of a separate sensor module, such as a general-purpose smartphone that may be held in a pocket on the garment, as previously described. Each garment may have an SMS (chip/microchip) that allows the garment to have connectors (female and male) with a numbers of pins (inputs/outputs) so that data from all the sensors in the garment (shirts, tights and accessories, such as gloves, socks, balaclava, etc.) may be first processed by the SMS and then sent through a connection (e.g., as few as 1 or 2 pins, or more) to the phone/communication module. In general, some of the sensors and components of the garments described herein may individually require multiple connections and thus a dedicated SMS may be very useful. For example, an IMU may require 5 pins and as many as 20 IMUs (or more) may be included as part of a garment, in addition to other sensors. Thus, the use of a dedicated SMS may allow the garment to manage a large number of data connections/contacts.

### Sensor Manager/Module

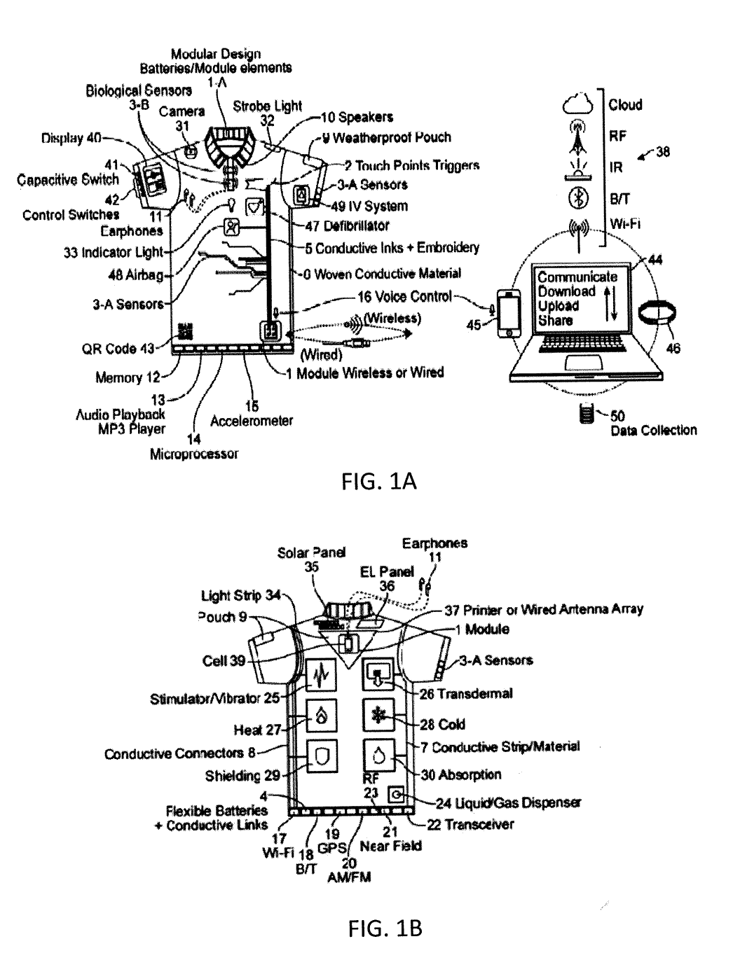

Any of the apparatuses (e.g., garments) described herein may include a sensor manager network (SMS network), that connects the sensors (including electrodes, etc.) on the garment to a processor, including in some variations a smartphone or other mobile device. The sensor manager system nodes and/or primary module may be a printed circuit board (PCB) that is part of the sensorized compression garment (e.g., shirt) and may be embedded into a rigid case placed on the shirt back, e.g., just under the neck as illustrated in FIGS. 1A, 1B and 2A and 2C. It is mainly responsible for collecting and elaborating the data coming from the sensors placed all around the shirt.

The sensor module may include different elements arranged on a PCB, such as a microcontroller (e.g., CY8C5 microcontroller (68 pin)) and all the connections with a phone module (metallized drill), tights (exposed solderable metal area) and sensors (connection with threads, e.g., conductive threads).

For example, electrical signals coming from the sensors may be carried by conductive threads sewed onto the shirt fabric or onto a tape (e.g., patch) made of the same material. All of these threads may arrive to the SM PCB and can be connected to it using connectors, or sewed/soldered around metallized drills. In contrast to the SMS illustrated here, an SM architecture in which sensors are connected directly to the Phone module would involve a relatively high number of pins (e.g., one for each trace/thread coming from the sensors). This may limit the number and type of sensors and could compromise the system stability. The architecture described herein allows connection of traces (e.g., threads) coming from the sensors directly to a microcontroller, using different types of connections that can be placed on the SM PCB. This way, all the sensors signals may be collected (aggregated) by the microcontroller, which will then communicate the processed data to the mobile processor (e.g., a smartphone) module by using only two pins, for holding a digital UART communication. This solution does not limit the type of number of sensors.

A Sensor Management System (SMS) may be located in the garment rather than on the module/phone. Thus, the number of pins remains constant even if the number of sensors varies between garments or accessories. For example the numbers of pins may remain constant (e.g., at 10-15) by adapting the specific SMS to generically work with different mobile processors (phones).

Also described herein are elastic electrical connector devices for incorporating into a garment to connect multiple electrical components in the garment, methods of making these elastic electrical connectors, garments including elastic electrical connectors and methods of making such garments.

An elastic electrical connector may be referred to herein as an elastic strip connector, a fabric strip connector, or the like. Generally, the elastic electrical connectors described herein may include a fabric substrate (e.g., cut or formed into an elongate strip of fabric substrate). This substrate may be elastic (e.g., it may be made of a stretchable fabric). A plurality of wires may be attached to one side of the fabric, and the plurality of wires may be attached in a sinusoidal (e.g., zig-zag) pattern along the length of the elastic electrical connector. For example, the elastic electrical connector may include a plurality of wires extending along a length of the first side of the elongate strip of fabric substrate in a sinusoidal or zig-zag pattern. The wires may be attached to the substrate by sewing or stitching. In some variations, the wires are attached by adhesive (instead of or in addition to stitching). For example, the plurality of wires may be attached to the first surface by one or more stitches at the peaks and troughs of the sinusoidal or zig-zag pattern.

In general, there may be spacing between the attachment points at the peak and troughs (e.g., between the stitches) holding the bundle of wires to the substrate in the sinusoidal or zig-zag pattern. This spacing may be greater than 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 20 mm, etc. (e.g., between about 1 mm and 15 mm); this spacing may be distance between durable attachment sites (e.g., stitches). The spacing between attachment points may along the length of the substrate may vary, or it may be constant. Leaving the bundle of wires (which may be twisted together) may make the wires easier to separate out for attachment to an electrical component as will be described below. Note that even in variations in which the wires are not referred to herein as attached, the wires of the elastic electrical connector may be considered as unattached, as the adhesive may not securely hold the wire(s) to the substrate between the peaks and troughs. In general, any of the variations described herein (unless otherwise specified) may include an adhesive on one or both sides of the elastic electrical connector, including the side to which the zig-zag/sinusoidal wires (wire bundle) is attached.

In some variations the adhesive may hold (or help hold) the plurality of wires or bundle of wires in the sinusoidal pattern as described. For example, the plurality of wires may be embedded within adhesive that holds (or helps hold) the wires in the sinusoidal (e.g., zig-zag, sawtooth, etc.) oscillating pattern yet allow individual wires to be removed from the adhesive and the substrate individually, e.g., by pulling, for cutting and attaching to an electrical device such as a sensor. In any of the variations including adhesive, the adhesive may help hold the plurality (e.g., bundle) of wires in the oscillating pattern along the substrate while still permitting individual wires to be removed from the side (e.g., back) of the substrate for attachment, leaving the other wires in the oscillating pattern. Thus, the adhesive strength (e.g., tensile or pull-off adhesive strength) of a wire held to the substrate (or within the substrate) may be relatively low, allowing it to be manually removed without damaging the individual wire or disrupting the oscillatory pattern of the other wires on the substrate.

Each of the wires of the elastic electrical connector may be electrically insulated. In particular, the insulation layer on the wire may be thermo-removable, so that just heating (e.g., by soldering, e.g., greater than 200 degrees C., greater than 250 degrees C., greater than 300 degrees C., greater than 350 degrees C., greater than 400 degrees C., etc.) may remove the insulation from the wire at the heated portion, leaving the rest of the wire(s) insulated.

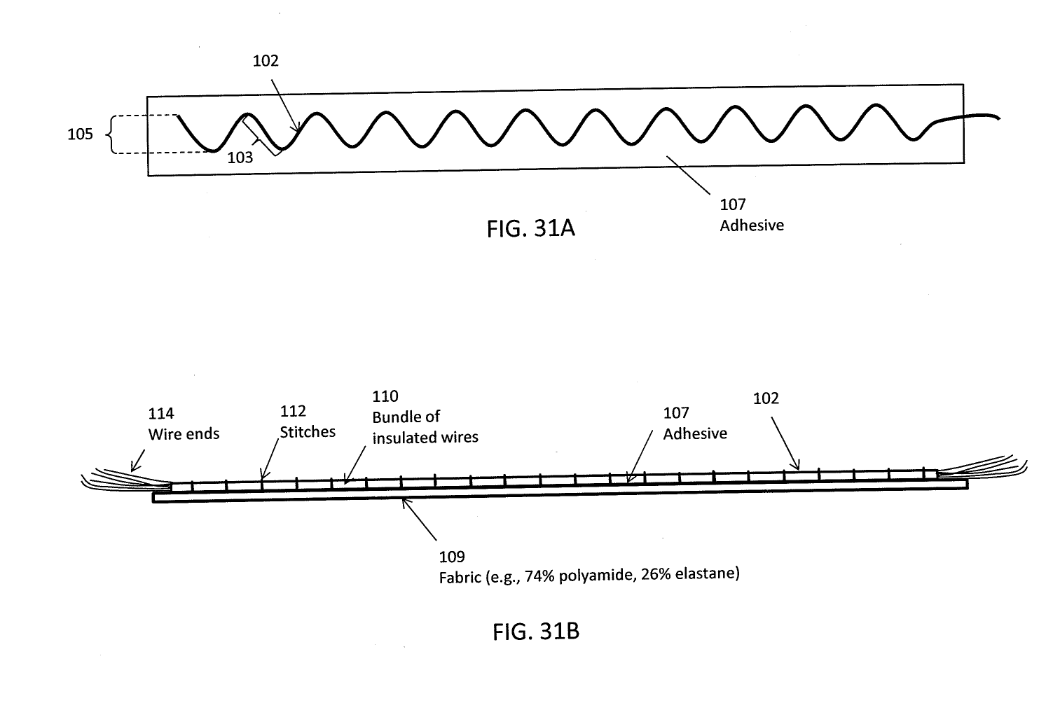

For example, FIG. 31A illustrates, schematically, one variation of an elastic electrical connector. In this example, the elastic electrical connector device includes a strip of elastic fabric 109, an adhesive 107, and a bundle of wires 102. Any number of wires (e.g., between 2 and 20, 2 and 19, 2 and 18, 2 and 17, 2 and 16, 2 and 15, 2 and 14, etc.) may be included in the connector device. In this example, the zig-zag/sinusoidal bundle of wires may have an amplitude 105 (from trough to peak) of between about 0.5 to 15 mm (or more, e.g., 17, 18, 19, 20, 21, 22, 23, 24, 25, etc., mm). The stitch length 103, or distance between trough and peak along the wire(s) may be between about 1 mm to 15 mm.

The electrical connectors described herein may allow deformation (elongation, twisting, curling, etc.) of the electrical connections. Shortly, this is achieved by embedding a bundle of electrical wire in a fabric sandwich held together by the thermo-adhesive. The thickness of the finished spidon may be important for wearable comfort. For example, the thickness applied may be between about 0.5 and 2 mm. (typically <2 mm).

Because of the arrangement of the zig-zag (sigmoidal) assembly may have material property advantages. For example, maximum elongation (which is dictated by the mechanical properties of the chosen substrate fabric) may increase. The geometry of the ZIG.ZAG pattern is optimized to ensure maximum elongation of the fabric in the long direction (Zig-zag direction) (i.e., the ZIG-ZAG is not the weak-link).

The amplitude and stitch-lengths of the patterns used to form the elastic electrical connector. For example, the device (e.g., elastic electrical connector) may be optimized to meet the above constraint and to support 3000 stress cycles, e.g., having a guaranteed elongation: of between about 80% to 400. The values for range of angles between the lines of wire extending between peak and trough of usually between 30 and 110 degrees.

The substrate used may be any appropriate substrate. For example the material used may be, e.g., Lycra, and other synthetic fibers. For example in some variations the fabric comprises a mixture of fabrics, such as a mixture of a synthetic (e.g., polyester) and another material (e.g. Lycra or elastin), e.g., around 25-40% of elastin or Lycra with the remainder being polyester. The fabric in some ways acts as a limiter, limiting the maximum stretch of connector to the maximum stretch of the fabric used, or less.

As mentioned, any appropriate glue (adhesive) may be applied to the back of the elastic electrical connector. For example, the adhesive may be applied to a thickness of between about 20 and 300 microns (e.g., between about 80-100 microns, between about 50-200 microns, between about 100-200 microns, etc.)

As will be described in more detail below, to connect a wire to an electrical component, the wire maybe cut and removed from the bundle at the cut end so that it can be electrically connected. The wires may be coded (e.g., color/pattern coded), and the proper wire may be cut (e.g., with a scalpel or scissor) and then when soldered directly; the application of the solder (heat) may remove e.g., by evaporation, the insulation. In general, the wires in the bundle are not fused or enclosed together, but may be secured as a bungle only at the apexes (peaks and troughs) of the sinusoidal pattern, e.g., by a stitch. This may allow the wires to be individually separated and pulled out of the bundle (and out of the stitches holding the pattern, e.g., by pulling the cut end from the bundle, allowing them to be easily identified and attached to an electrical component, such as a sensor or PCB.

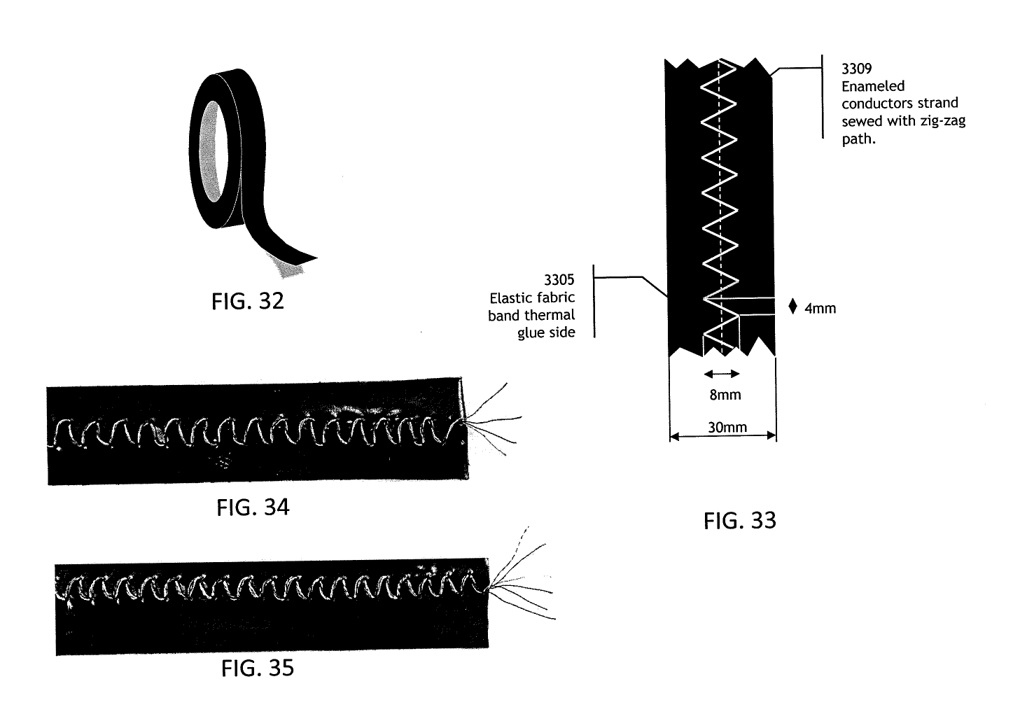

Overall, the strip of fabric forming the device may be cut into fabric strips of any length and width. E.g., strips may generally be between 3-4 cm widths (e.g., as thin as possible). Likewise, the length may be varied. In some examples (e.g., FIG. 32), a roll of elastic electrical connector may be made and cut to order during fabrication of the garments described herein.

This elastic electrical connectors may also be referred to as fabric ribbons or fabric ribbon connectors, and may include the conductive zig-zag (e.g., sinusoidal) enameled, twisted wires. The purpose of the elastic electrical connector is to deliver signals and electricity in every needed part of a garment. There are numerous advantages to this type of elastic electrical connector: every single wire/conductor can be easily connected to a sensor, an electrode or an electronic board without having to strip the wire's jacket, or remove the fabric protection or others. This is possible because the strand on enameled, twisted wires (composed from 2 to up to more than 8 wires) is sewed on the glued side of the ribbon and can be easily worked on (cut, stripped of protection, welded, attached, . . . ) before being thermally applied to the garment. Therefore only a single simple operation is needed in the production process: removing the cut wire's insulation so that it can be welded to electrodes, sensors or any electronic or electrical parts.

Moreover, this allows us to prepare the “harnesses” with all the required connections in advance, to test it and to then ‘attach’ it (the ‘harness’ or SPIDON assembly) to the garment in one single/efficient/low-cost operation much like is done in the car manufacturing for the electrical distribution. In contrast to other devices and methods for connecting electrical components on a wearable garment, the elastic electrical connectors described herein are relatively thin (e.g., less than 2 mm, less than 1.9 mm, less than 1.8 mm, less than 1.7 mm, less than 1.6 mm, less than 1.5 mm, etc.). In contrast, other connectors are too thick which may prevent the comfort needed in compression or tight clothes. Other connectors are also described as woven inside the ribbon, thus the connections can only be done at the beginning or at the end of the ribbons so many different ribbons are needed. Further, it may be very difficult and time consuming to cut the ribbon at the desired dimensions and strip out the wires without damaging them. In some cases the wires may not have insulation, thus they have to be sewed separately limiting the ribbon width to the number of wires, moreover the ribbon risks to generate short-circuit effects when in contact with sweat or rain.

The fabrication of the conductive ribbon as described herein may start with the coupling of a thermo adhesive film with the fabric: the two coupled materials pass then between two hot metal rollers that melt the glue onto the fabric side. A fabric reel normally has a dimension of 140 cm width and a length of about 70 m: after the glue coupling process, the reel can be cut in smaller reels sized to the desired width (FIG. 31A-31B). The ribbon reels come out with fabric on the external side and glue (protected with silicone-paper film) on the internal side.

Using a special custom designed sewing machine, the conductors strand is sewn over the glue side of the ribbon (FIG. 33) after the protection film is removed. The sewn ribbon has a standard length from 5 up to 8 meters depending on the size of the spool and the capacity of the sewing machine as well as on the number of wires inside the strand being used. Depending on the application, the strand can be sewn in the center of the ribbon (FIG. 34) or on one side (FIG. 35). The center sewing is normally used for UART BUS distribution where a local uP on board of a PCB (FIG. 36) or an external connection (FIG. 37) are needed.

The side sewing may be used for sensors (FIG. 38, showing a strain gauge) and electrodes (FIG. 39) connections in the copper adhesive pads in order to use the free space of the ribbon for cover and seal the contact area.

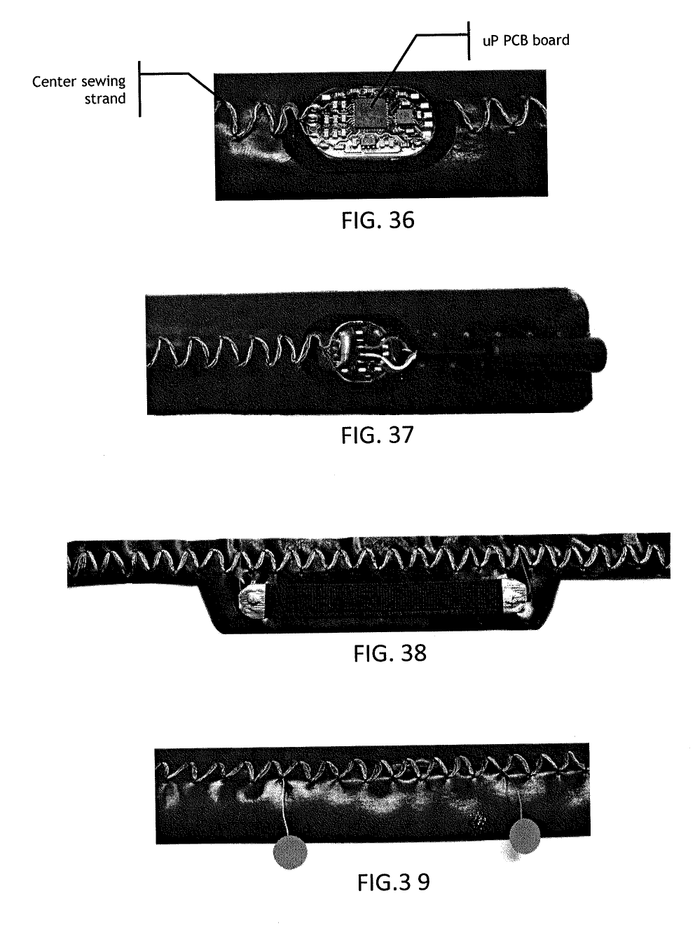

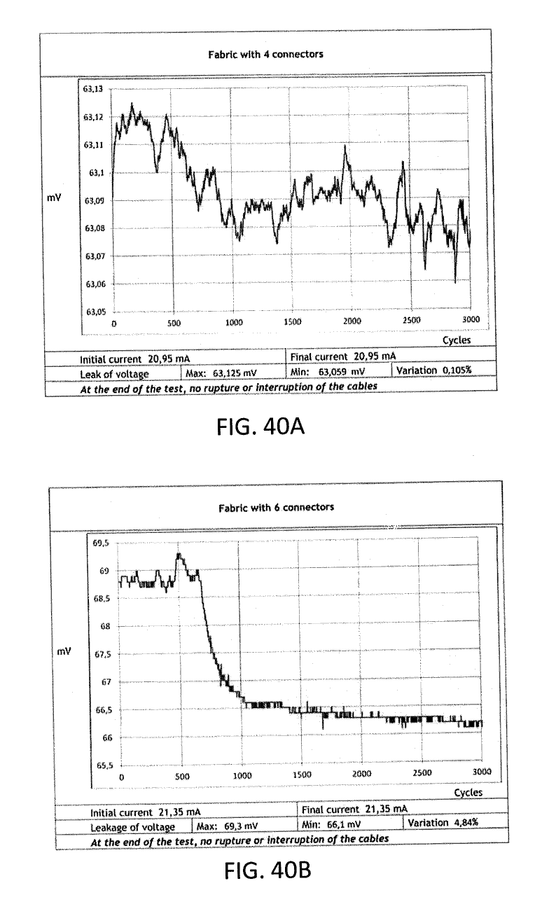

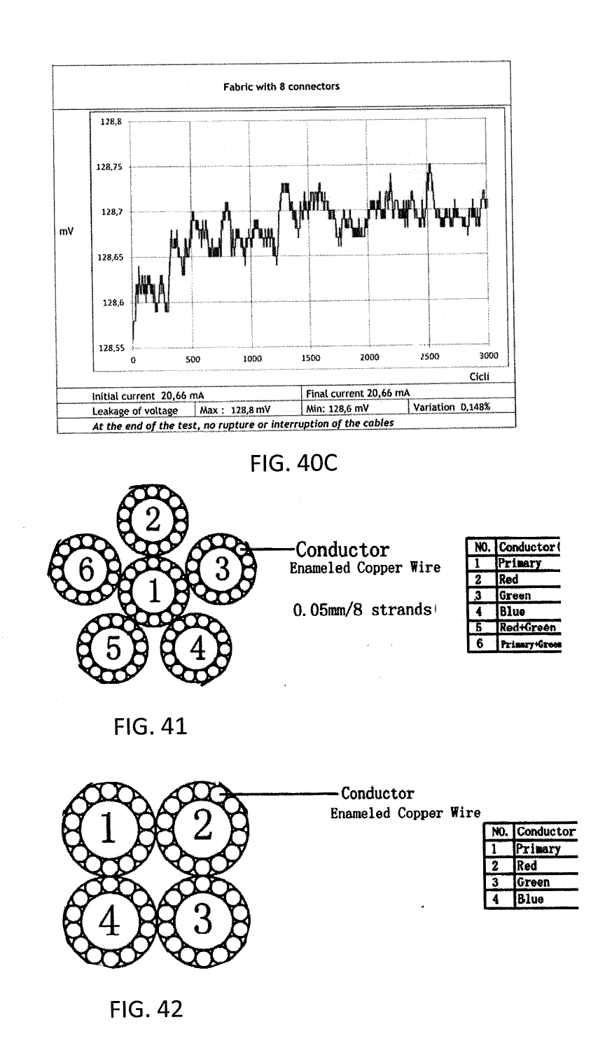

The use of an elastic electrical connector as a garment electrification method has been tested by an external certified laboratory with a cycling test bench machine doing a tensile strength with 20% of elongation to verify the electrical continuity of the conductors. Note that other (e.g., 30%, 40%, 50%, 60%, 70%, 80%, 90%, 100%, etc.) elongations have been successfully tested with similar results as well. FIGS. 40A-40C illustrate these results, which verify that these devices have a surprisingly high reliability and effectiveness.

Each single screen test has been applied by thermal transfer onto a piece of elastic fabric (same fabric material of the ribbon) with dimensions of 45×20 cm. One end of the sample was bound to the frame of the test device, while the other end was fixed to the pneumatic piston. The electric wires of the samples, connected in series each other's, have been connected to the source of direct current power supply through a current-limiting resistor. Potential voltage leak at the ends of the wire was monitored by means of a data logger. The tests have been conducted on three different screen test samples: one with 4 conductors, one with 6 conductors and one with 8 conductors.

TABLE 1
TEST PARAMETERS

| Distance between jaws | 270 | mm |
|---|---|---|
| Elongation | 20% | |
| Cycle frequency | 1 | Hz |
| Number of cycles | 3000* | |
| Voltage acquisition frequency | 1 | Hz |

*Note: the number of cycles have been calculated considering one ‘dressing’ and one ‘undressing’ per day, for a product life duration exceeding 4 years (as a comparison standard washing cycles are between 40 and 50 times).

FIGS. 41 and 42 schematically illustrate cross-sections through six and four strand wire bundles. The wires in this example are all enameled copper wires that are 0.05 mm/8 strands thickness. These wires may each be individually color and/or patterned coded, as indicated by the numeric keys to the right of each figure.

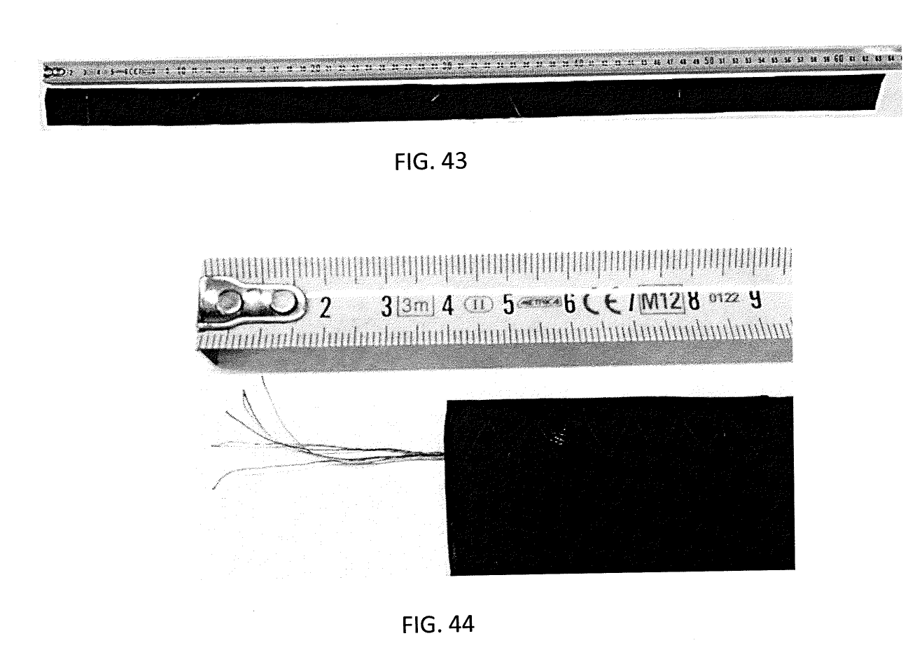

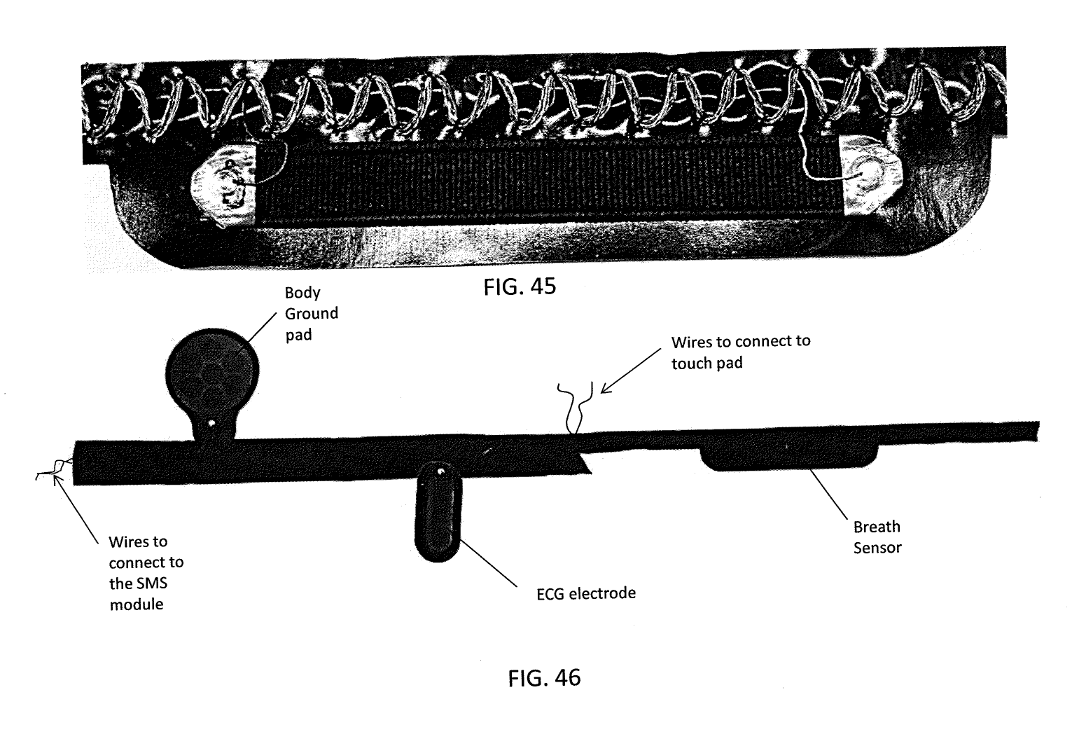

FIG. 43 illustrates an example of a length of elastic electrical connector, shown as long, relatively thin (e.g., between about 2 and 5 cm) and relatively flat (e.g., less than 2 mm). FIG. 44 shows an enlarged view of just the distal end, with the wires (six are shown) exposed. FIG. 45 is an enlarged view of a portion of an elastic electrical connector shown connected to a sensor, specifically a stretch sensor. In practice multiple electrical components (including sensors, PCBs, microphones, electrodes, speakers, etc.) may be connected by the elastic electrical connectors described herein. For example, FIG. 46 illustrates an elastic electrical connector showing multiple electrical components electrically connected to the wires of the elastic electrical connector.

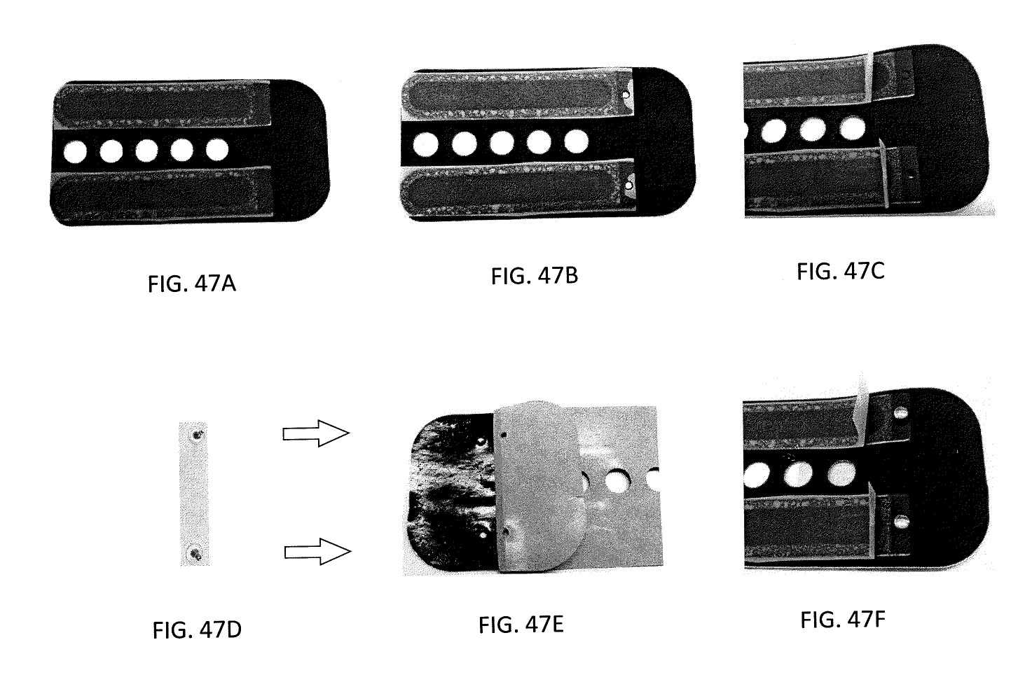

FIGS. 47A-47F illustrate one method of making a sensor that may be connected to the elastic electrical connectors described herein.

As mentioned above, the connectors described herein may be part of a system including one or more flexible connectors (which may be referred to as a “spidon”), that may connect multiple electrical components, including connecting such components to a Sensor Management System (SMS), having male and/or female connectors with their components. The spidon may be configured as a harness with multiple intelligent strands (e.g., made of twisted enameled multi (2 to 20, 2 to 18, 2 to 16, 2 to 14, 2 to 12, etc.) wires sewed against one side of a fabric strip in a sinusoidal (e.g., zig-zag) pattern, and may include isolating glue. The spidon may connect and therefore include electrodes, sensors, haptic actuators, touch-points and ICs such as microcontrollers and IMUs. A spidon may be designed for garment application where signals coming from multiple sensors, electrodes, touch points and haptic actuators placed in different parts of the garment/body have to be connected to microprocessors placed in different parts of the garment/body and to (an) external devices such as a Multi Media Module device (MMM). The SMS connector is part of the Spidon and may be positioned in the upper center of each shirt, which corresponds to the center between the wearer's shoulder blades, the place in the human body less sensitive to weight and to touch.

The SMS may be placed in each shirt rather than in the MMM. This solution increases the cost of the system: rather than buying an MMM with a SMS and use it with many shirts with no SMS, the user now has to buy a MMM without an SMS and use it with many shirts each one having an SMS. However this solution allows to increase the number of sensors, electrodes, touch points and haptic actuators in the garment without having to increase the size of the male and female connectors on the MMM. Potential users may not wear an SMS with more than 36 pins because its size would become too intrusive and uncomfortable.

By placing the SMS in the connector glued to the shirt, each sensor, electrode, touch point or haptic actuator may be directly connected to the SMS microprocessor through the already mentioned strands. The SMS microprocessor is then responsible for acquiring and processing each sensor, electrode, touch point or haptic actuator data and signals, and for sending those calculations to the MMM through a digital serial port that requires just two pins on the SMS connector.

It should be noted that in case the SMS would have been placed in the MMM rather than in shirt connector, all the sensors, electrodes, touch points or haptic actuators would have been connected to the MMM, thus dramatically increasing the number of pins on the connector and, as a result, increasing its overall size. In this case, a high number of sensors, electrodes, touch points or haptic actuators could be achieved only at the cost of a bigger connector size. On the contrary, the chosen solution ensures small connector dimensions and a high number of sensors, electrodes, touch points or haptic actuators (up to forty-four connections or more) at the same time.

In addition to the already described architecture, additional technology allows the system to increase the number of sensors, electrodes, touch points or haptic actuators without increasing the number of strands that need to be embedded into the garment and connected to the SMS connector. This may be achieved by using the intelligent dedicated strands that were already mentioned above. These intelligent strands which connect embed sensors, electrodes, touch points, haptic actuators and microprocessors that communicate with the SMS microprocessor in a similar way as the MMM and SMS are. Each bundle may include multiple strands or wires. For example, four twisted enameled wires may be used: two wires to carry signal (e.g., acting as a digital serial communication bus), and two for the power supply and ground.

Following a similar principle as the one described for the SMS, it is possible to consider additional ‘modules’, each containing one or more, each additional microcontroller, embedded into intelligent strands can be then connected to a high number of different sensors, electrodes, touch points and haptic actuators placed on the garment. These modules are connected to the SMS by the strands. The microcontroller, in fact, manages not only sensor conditioning, but also digital communication.

In addition to this first advantage, there are two other important features that should be noted. First, by using this overall system architecture, the number of wires that go around the garment is considerably reduced, because the sensors, electrodes, touch points or haptic actuators are not connected to the SMS but to the microcontrollers, and also because all the microcontrollers can share the same digital serial bus for communicating with the SMS microcontroller. This is possible because each microprocessor is identified by an address, thus it can be uniquely identified while communicating with the SMS. The fact that the number of wires is reduced by this solution, surely improves garment wearability and comfort for the final users. Wearability and comfort is important for wearable computers like ours that operate when in direct contact with a large portion of our skin (entire upper body/shirts, entire lower body/tights, hands/gloves, feet/socks, head/balaclava and more) contrary to computers, smart phones that are used while on desks, in hands or in pockets or intelligent watches or wrist bands that are worn on our wrists.

It should be also noted that the number of different microprocessors that can share the same digital serial bus is theoretically infinite or very high and limited primarily by the space on the garment and by the computational power of the SMS microcontroller that needs to manage all the microcontrollers placed around the garment (including the interrogation frequency, bandwidth, etc.).

Lastly, the combination of microcontrollers, sensors, electrodes, touch points or haptic actuators connected to it, allows to create a sort of “smart sensorized node” that can be managed independently from the SMS and can help to distribute the data processing and to relieve the SMS microcontroller processing load.

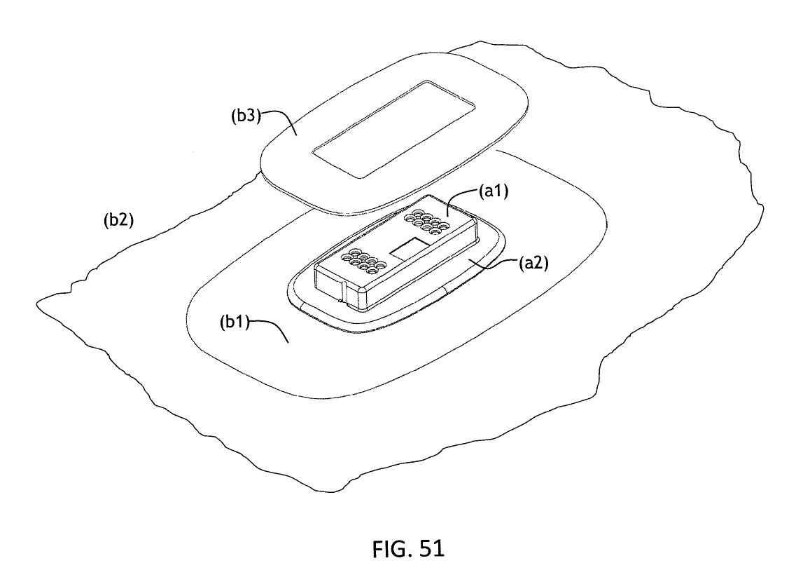

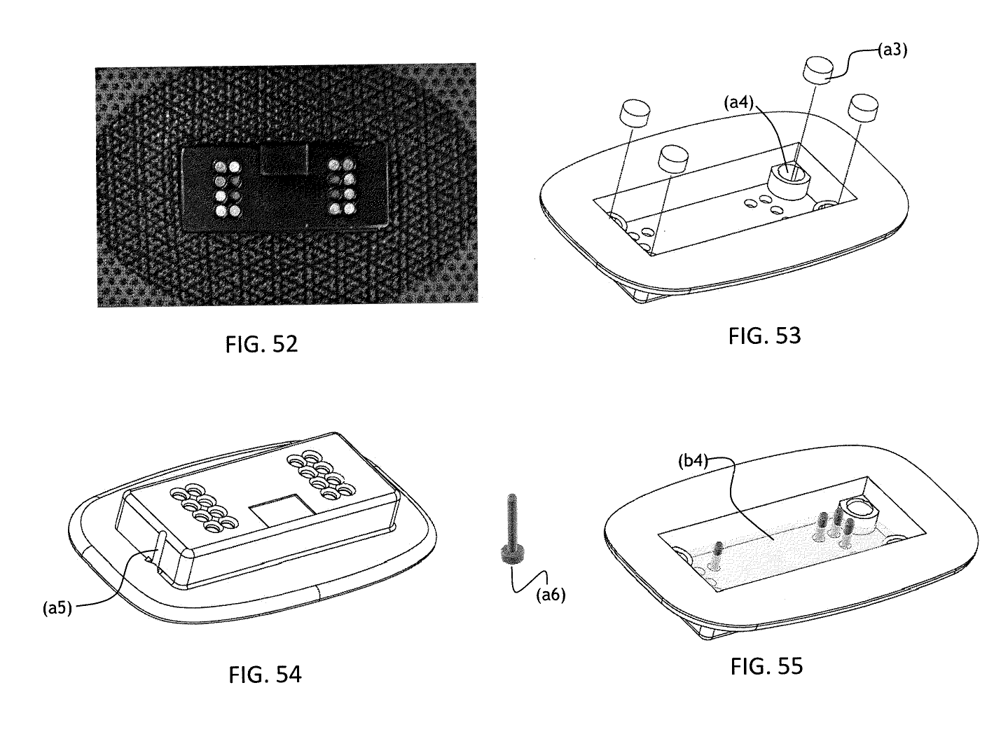

Referring to FIG. 51, the connector body (a1) is made off polycarbonate plastic material with an overall thickness of 2 mm in order to guarantee shock protection. At the base there is a flat flange (a2) which allows a good stability on the soft plastic layer support (b1) which is hot-melted to the garment fabric (b2). One additional purpose of this flange is to fix the connector to the first plastic layer through an additional layer (b3) which is hot-melted to the flange in order to ‘sandwich’ the connector to further stabilize it. The final assembling is shown in FIG. 52.

In FIG. 53, the SMS connector has four magnets (a3) placed at the four corners cylindrical seat (a4) that allows to lock easily and to stabilize the external device to be connected. This connector has a 68 IP grade to be completely waterproof to endure regular washing (it is an ‘intelligent’ garment thus needs to be regularly cleaned after use), thus the magnets are positioned at the back side of the surface in order to avoid possible oxidation and rust deposit.

In FIG. 54, despite the matching position between the external device and the SMS connector is constrained, on both sides right and left, is present a semi-cylindrical slot (a5), designed to avoid unwanted reversed connections.

The connector may also be made waterproof, e.g., or at least water/moisture resistant, as shown in FIG. 55. The female contacts receptacle has been designed to avoid any water access to the inner parts. After the female contacts (a6) insertion in the corresponding pinhole, the back side of the connector is filled with epoxy resin (b4) in order to seal completely any interstice.

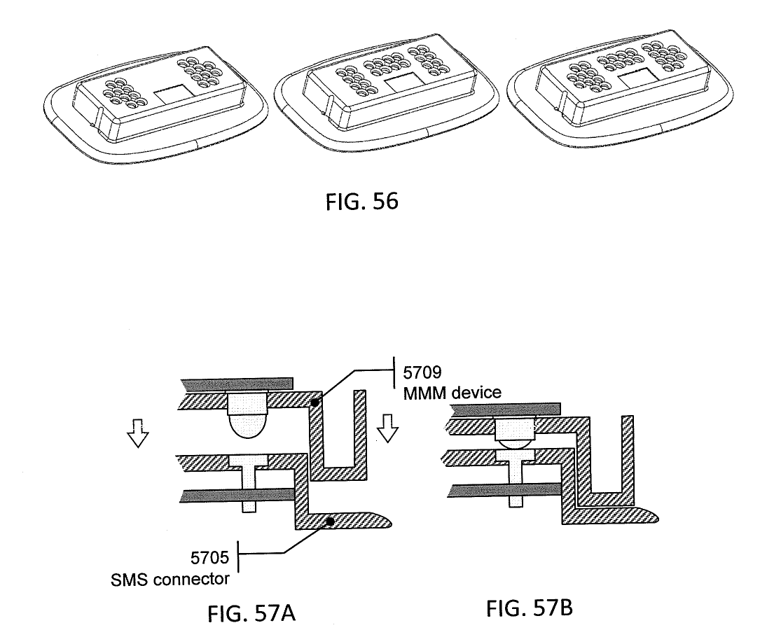

One basic pins configuration is shown in FIG. 54 with 12 poles, but it could also be configured with 16, 20, 24 and 28 poles as shown in the variations of FIG. 56 (showing, 20, 24 and 28, respectively).

FIG. 57 illustrates the electrical connections between the SMS (Sensor Manager System) connector and the external Multimedia Module device (MMM) are through female contacts (a6) and pogo pins (b5) that thanks to the internal spring ensure stable and reliable electrical contacts even under extreme shocks and vibrations.

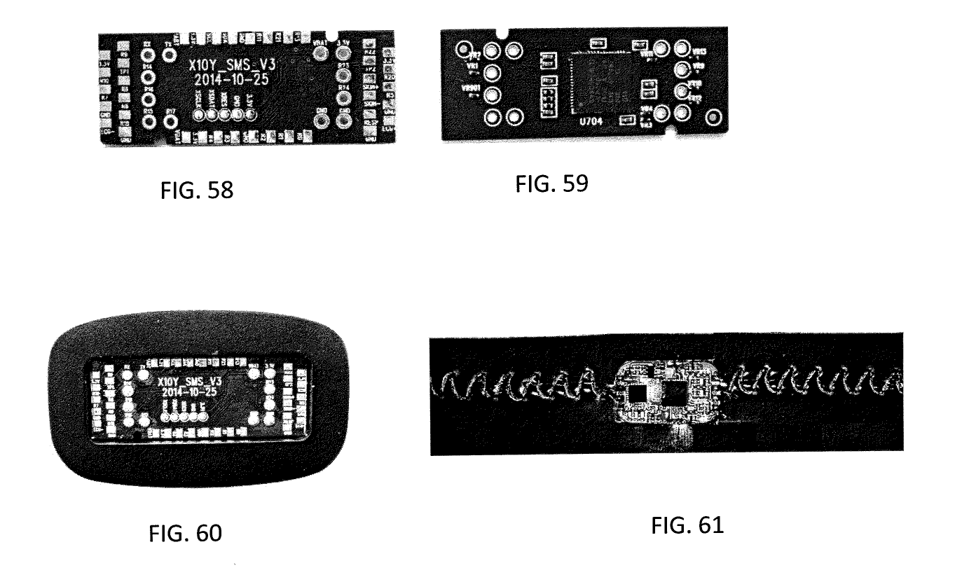

FIGS. 58-61 illustrate an SMS. The SMS shell shown contains a printed circuit board assembly (PCBA) (FIG. 58), directly soldered to the female contacts and acting as a central unit able to acquire and process data and signals from sensors, electrodes, touch points or haptic actuators embedded into the garment and transmit them to the MMM.

The SMS main component is a microcontroller. As already mentioned, the main purpose of this microcontroller is to manage the acquisition of data and signals coming from sensors (e.g. ECG electrodes, EMGs, string gauges, skin conductance, IMUs, etc.), electrodes, touch points or haptic actuators. The same component is also involved into a first phase of data processing (e.g. digital filters) and into the communication of these calculations to the Multimedia Module through a serial digital line.

In one example, shown in FIG. 59, twelve pins (female contacts) ensure the electrical connection. These pins are used for having the digital communication, the power supply coming from the Multimedia Module battery (regulated +3.3 V and protected VBAT) and other hardware features (e.g. sensing of the connection between Multimedia Module and SMS). In FIG. 60, the SMS PCBA solder layer has been designed to allow several connections to various types of sensors, electrodes, touch points or haptic actuators distributed throughout the garments to cover the body specific parts (arms, hands, legs, feet, shoulders, head, thorax, back, abdomen, etc.) through a special harness made with elastic ribbon to which is sewed a strand of 2 to 12 (or more) enameled conductors/wires. The strand (bundle of wires) is sewn at the peak and trough of the zig-zag pattern, with each side in this example measuring from a minimum of 2 mm to a maximum of 4 cm and with angles between 10 and 179° in order to allow the ribbon to stretch from 10% to 500% of its length. The ribbon band is made with the same fabric utilized to make the part of the garment (sleeve, shoulder, etc.) where it is applied. Since stretchable fabrics stretch in various directions (from 1 to 6 or more) the ribbon is applied following the exact stretching direction of the part where it is applied. This process ensures that the ribbon has the same elongation and the same return as the fabric where it is applied to improve functionality (conductivity and data collection) and comfort in wearing the garment. It also improves the looks of the garment (seamless stretching and return of the garment when body is in motion). The elastic ribbon is glued to the stretchable fabric through an adhesive film especially formulated for fabric applications, this adhesive is on the same wiring layer and it is used for hot fixing to the garment's elastic tissue in order to block and keep the zig-zag strand shape after hot application. The zig-zag shape has been optimized to assure the wires elongation during donning and usage avoiding the mechanical stress of the copper conductive material.

In any of the connectors described herein in which the insulated wires are sewn onto the substrate, a separate thread material (e.g., cotton, polyester, blend, etc.) may be used to sew the bundle of wires against the substrate (fabric) at the appropriate regions. A single loop of thread, or multiple loops of thread may be used to hold the wires in place. The thread may pass around the bundle of wires one or more times, and through the substrate one or more times. The stitches securing the wires to the substrate may be separated by a spacing distance (e.g., see FIG. 31, element 103).

The wired elastic ribbons connect different sensors types as: IMUs, EMGs, electrodes, touch points, ink sensors by conductive washers connections (e.g., FIG. 31), haptic actuators, PCBA (FIG. 40) and any kind of electrical connections.

In case of PCBA incorporation, this must be previously covered by epoxy resin to prevent any water, sweat or any kind of liquid penetration inside the electronic circuit. The coverage has a smooth and rounded shape in order to have a good touch feeling and an attractive appearance from the external side of the garment

The conductive washers may be used for connect the copper wire, soldered on it, to the ink sensors and are made by silver-chloride thin steel film in order to have a strong bending resistance and good protection against rust and oxidation, maintaining optimal conductivity values. The coupling between the washer and the ink surface is made thanks a special conductive adhesive named z-axis (manufactured by 3M) that allows transmission of electrical signals between the two different material surfaces.

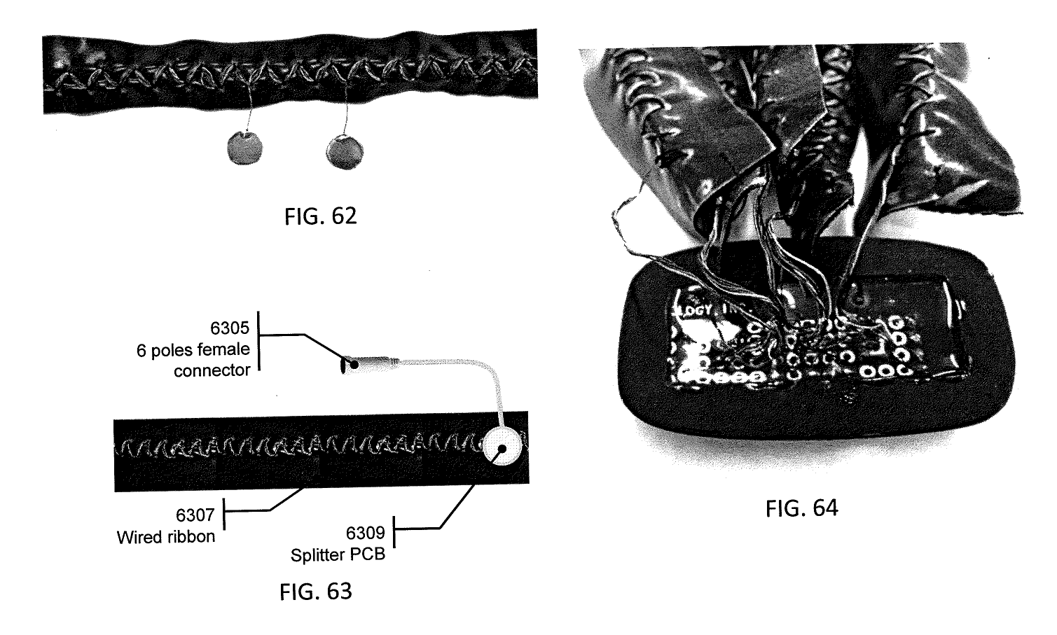

With this system, it is possible have also input/output electrical connections, like connectors or external modules in every parts of the garments thanks to the “splitter PCB” (SPP) that allows the connection of the thin enameled conductor to standard harnesses. As per the PCBA, the SPP must be protected by epoxy resin coverage after cabling. FIG. 62 illustrates a connector electrically connected to a pair of electrodes.

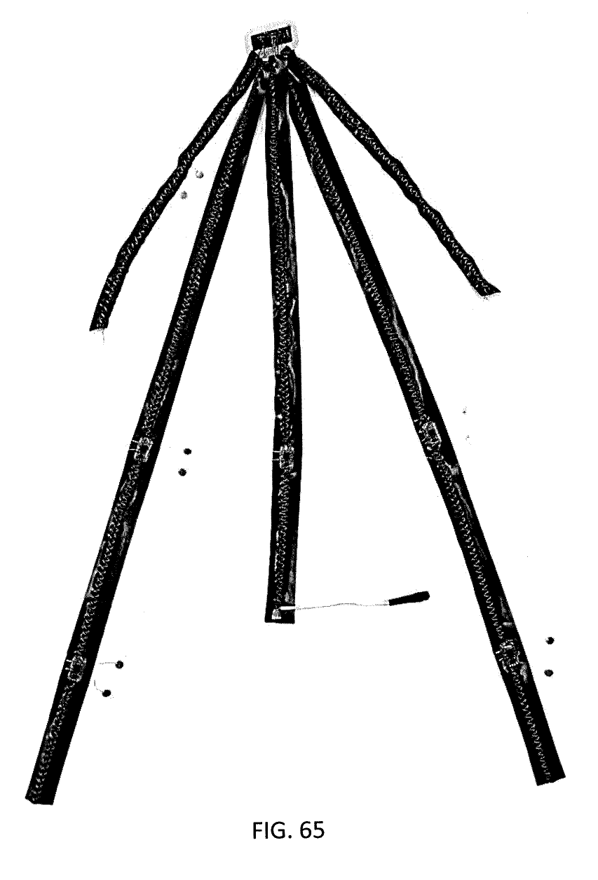

All the wired ribbons terminations are soldered to the SMS PCBA pads on the Solder layer and, after test, are incorporated by epoxy resin inside the SMS connector shell (FIG. 63) in order to completely prevent water penetration. A Spidon (e.g., FIG. 64, FIG. 65) may then be ready to be coupled with the garment. First of all the SMS connector may be inserted through a slot present on the high back side of the shirt and mechanical fixed to the garment as described above (e.g., FIG. 51), then following the draw projected by a laser projector, the various wired strips may be positioned to the right place of the internal garment surface and using a hydraulic press for thermo printing, fixed to the tissue.

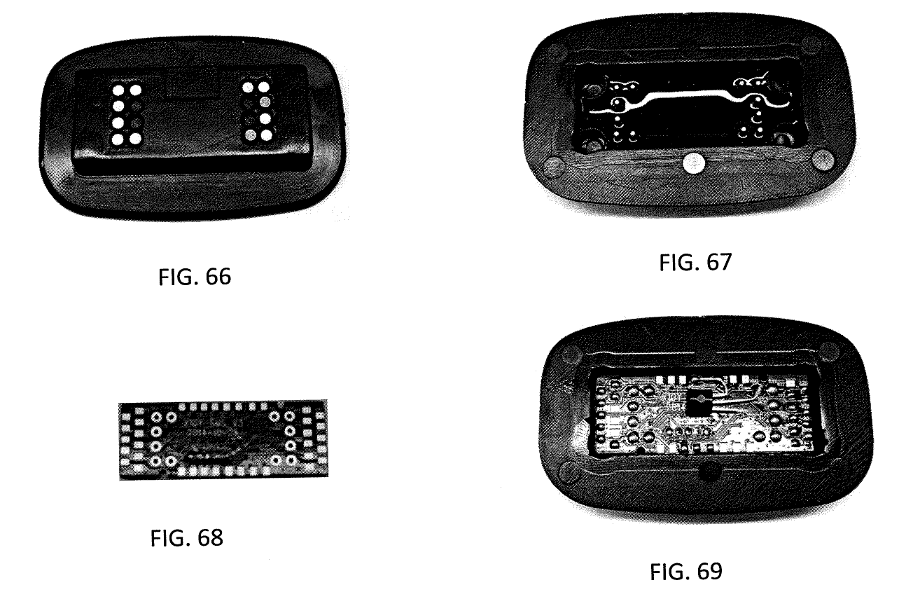

Other examples of SMS and SMS connectors are shown in FIGS. 66-69.

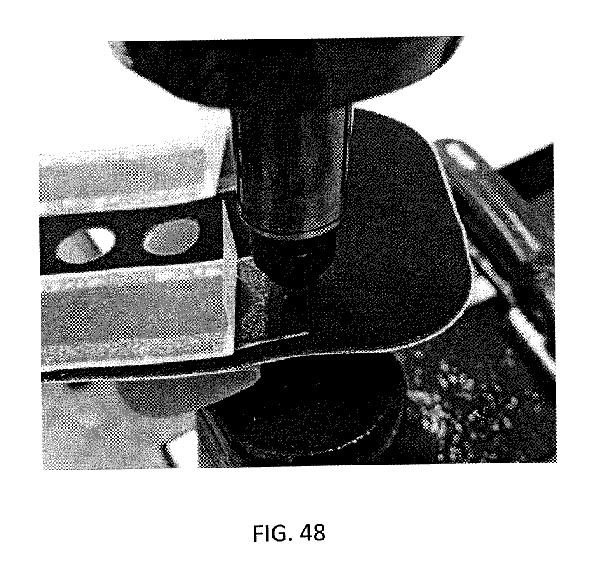

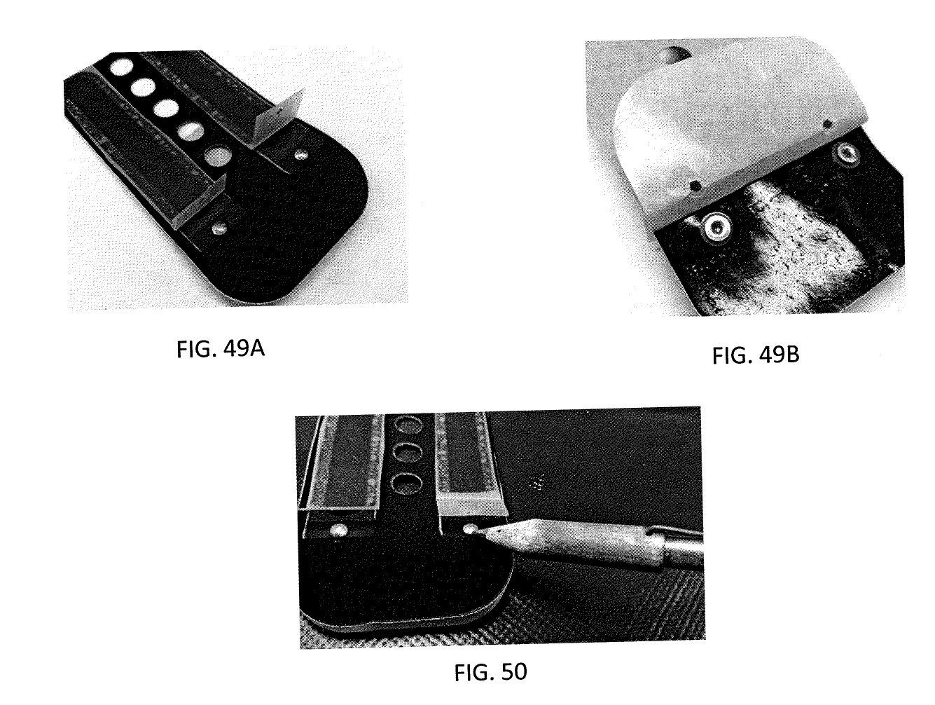

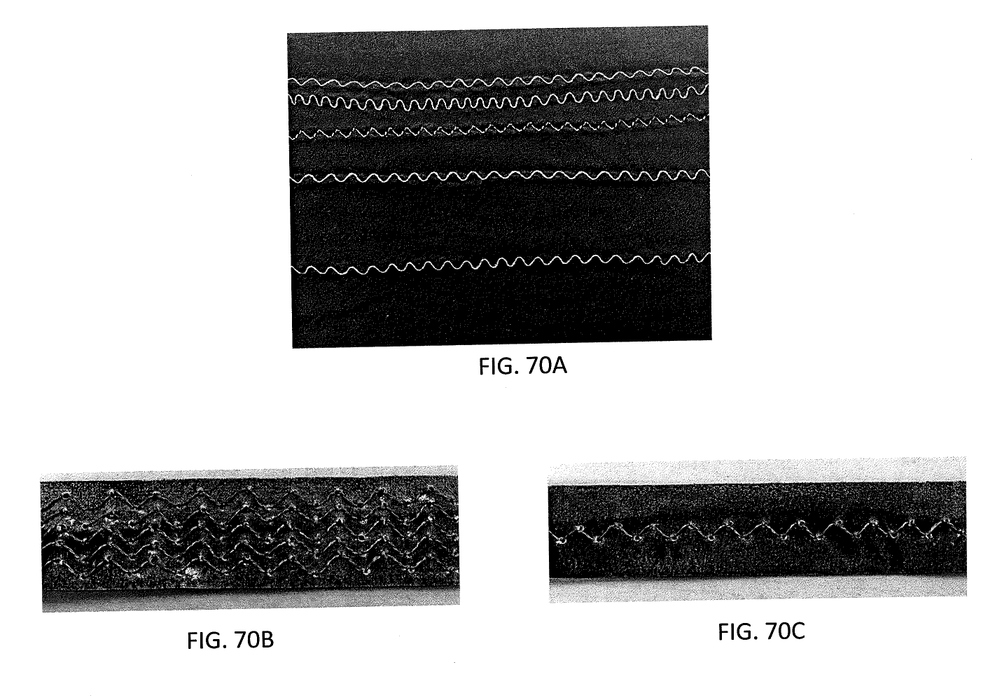

FIGS. 70A-70C illustrate other examples of flexible connectors having a wires attached in a sinusoidal or zig-zag pattern. In these example, one or a bundle of fibers is attached (including sewn into the fabric, rather than using an additional thread to sew the wires onto the substrate as described above in FIGS. 31-69). Thus, these embodiments may not have all of the advantages, including ease of removing and connecting a wire, as described above.

In some variations it may be useful to use conductive threads or other high-conductivity connectors, such as those shown in FIG. 70A-70C. In this example, the conductive thread is stitched onto the garment in a wavy (e.g., zig-zag, sigmoidal, etc.) pattern that allows some stretching in the net direction of the stitching. As described above, respiration (sensors) traces may be formed of stretchable conductive ink patterns to take advantage of the change in conductivity with the change in resistivity with stretching of the conductive ink pattern. In this example, the sewn pattern of threads includes an approximately 35-40 degree zig-zag pattern allowed the stitch to elongate slightly with the fabric. In some example, the conductive thread is a metallic conductive thread. The angle formed at each turning point (in the wavy pattern) and the width of the pattern may depend upon the textile used. In general, the higher the stretchability of the textile, the smaller the angle. The number of threads may vary; in general, any number of threads may be used depending, for example, on the number of sensors and their pins that need to be connected. The threads are typically sewn directly on the garment. The electrical insulation of the thread may be obtained by an external coating on the thread (e.g. silicone, polyester, cotton, etc.) and/or by a layer of insulating adhesive, as described above. The thread connectors may also be used as part of a transfer as described above. For example, a conductive thread may be sewn on a band made on the same fabric of the garment and then transferred by a thermal process to the garment, e.g., using a layer of adhesive.

One or more conductive threads may be applied directly to a fabric (such as a compression garment) or to a transfer (e.g., patch of fabric or other material that is then attached to the garment). Conductive threads may be insulated (e.g., enameled) before being sewn. In some variations the conductive thread may be grouped prior to sewing onto a fabric or other substrate. For example, a plurality (e.g., 2, 3, 4, 5, etc.) of threads may be insulated and wound together, then stitched into a substrate, such as the compression fabric. For example, in one variation, an apparatus includes a garment having an IMU and two EMGs with inputs fed into circuitry (e.g., microchip) on the apparatus, including on a sensor module/manager. The components may be operated on the same electronic ‘line’, where the line is a plurality of electrically conductive threads that are combined together for stitching through the substrate. In one example, two microchips can be operated by the same ‘line’ made of 4 wires, where each wire is electrically isolated from each other. In stitching a material, the stitch may be formed of two sets of wires; one on top of the substrate and one beneath the substrate, as is understood from mechanical sewing devices; in some variations a stitch formed of conductive thread may include an upper conductive thread (or group of conductive threads) and a lower conductive thread (or group of conductive threads), where the upper conductive thread(s) is primarily on the upper surface and the lower conductive thread(s) are primarily on the lower surface (but one or either may pass through the substrate to engage with the other).

For example, a conductive thread may include a very fine (e.g., 0.7 millimeters gauge/thickness) ‘wire’ made of 4 twisted and enameled (thus electrically isolated from each other) wires covered with a binding solution (that is silicon or water based) or protected by a jacket, having a total diameter of about 0.9 millimeters. A conductive wire may be sewn in a wavy (e.g., zig-zag) pattern, such as a pattern having 45 to 90 degrees angles between the legs of the zig-zag, directly on a fabric or substrate. In some example, the pattern is formed on a substrate of material (e.g., fabric) and attached to the garment. For example, the substrate may be a 1 cm to 3 cm self-adhesive strip of fabric.

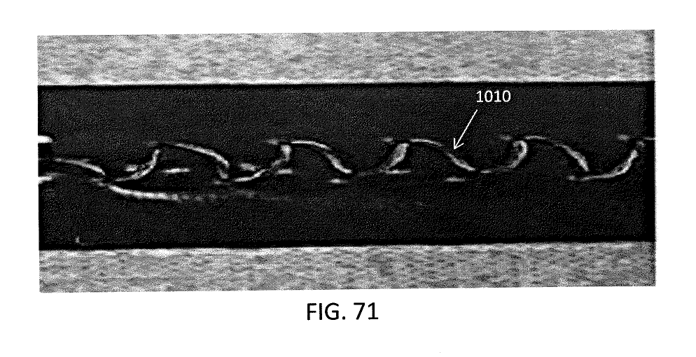

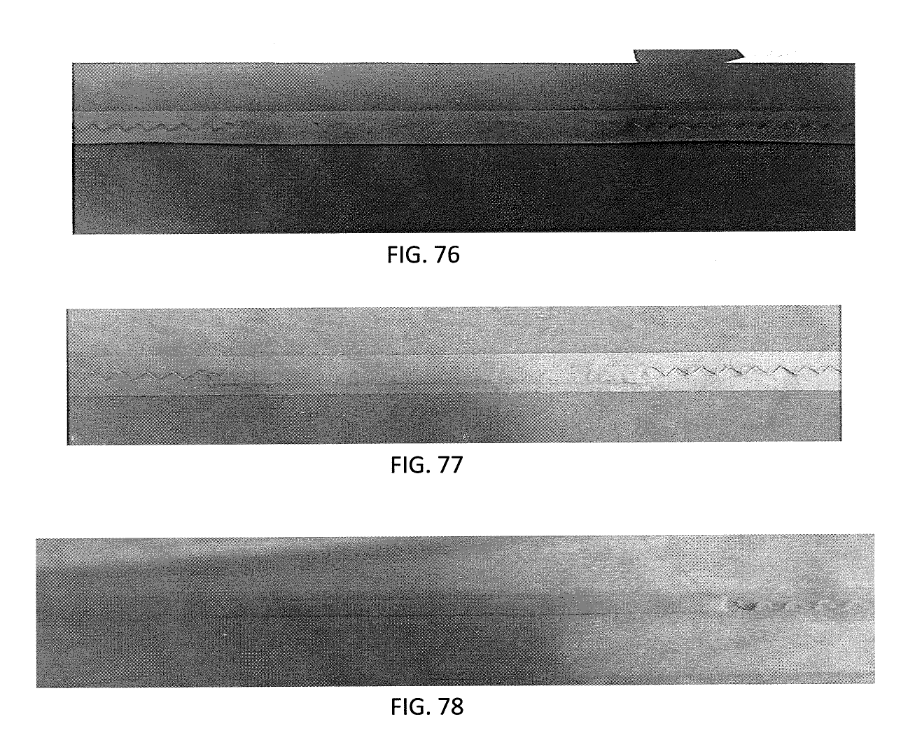

FIGS. 71-78 illustrate the connection and formation of one type of sensor to an elastic electrical connector as described above. In this example, a stretch sensor may be formed by impregnating an elastic material with conductive particles, allowing it to dry and then coupling contacts at the ends, to form terminals. Once the terminals are attached, the elastic material may be coupled to a wire connector, such as the pre-prepared wire ribbon material shown in FIG. 71. In FIG. 71, a wire ribbon material is sewn into a strip of fabric with a pair of twisted wires 1010 (though more than two wires may be used), shown as twisted, enameled (insulated) wires. The wires are sewn into the strip of fabric (e.g., compression fabric) in a zig-zag pattern and the fabric strip may include a fabric adhesive or may be configured for thermally applying to another fabric (e.g., garment), so that the conductive connectors can be applied directly to the fabric without having to sew directly onto the fabric, and providing a covering for the wires. The fabric onto which the wires are sewn is typically the same material to which they are to be applied (e.g., a compression garment fabric). In some variations one side of the fabric onto which the zig-zag pattern of insulated wires is sewn, which may be referred to as an applicator fabric, include or is treated for use with a fabric adhesive (including thermally active adhesive). In practice, long lengths of wire may be prepared ahead of time and cut to need for application to a garment Note that in general, a wire ribbon material may be used as an electrical connector connecting one or more sensors to other portions of the garments described herein, including a data module, and/or an SMS component. This wire ribbon material may be referred to herein as a wire ribbon material or as a stitched zig-zag connector. This material may be advantageously prepared in long lengths and cut to the desired length for securing (e.g., adhesively securing) the garment and/or sensor.

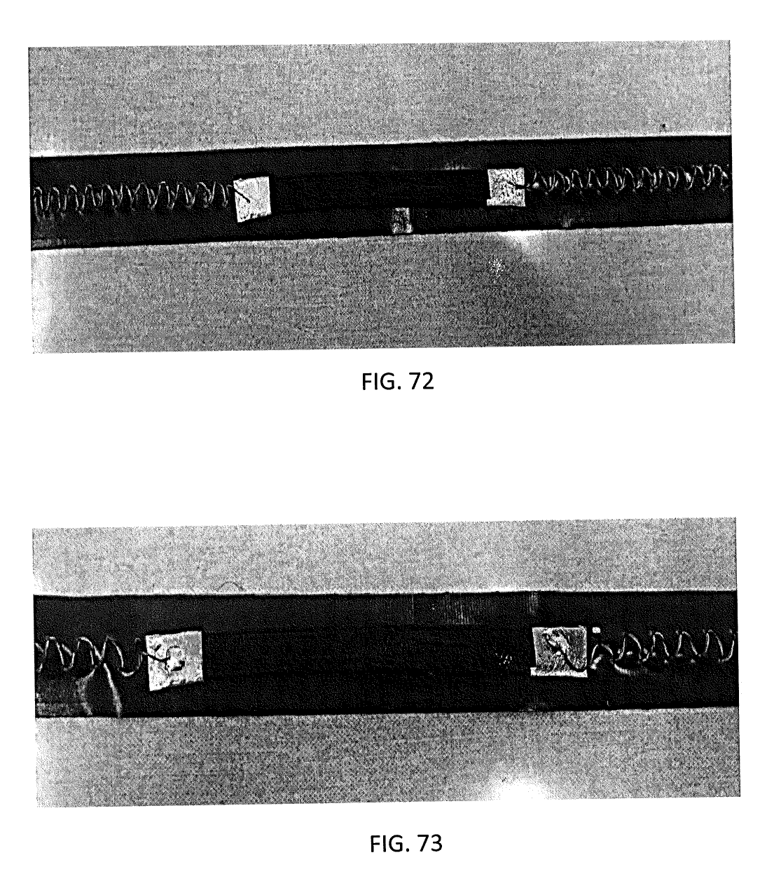

For example, in FIG. 72, the conductive elastic ribbon is place on a thermo adhesive glued surface of the wired ribbon in a region that does not include wire, and connected to the conductive wire ends. For example, as shown in FIG. 73, the conductors (wires) are soldered to the copper terminals.

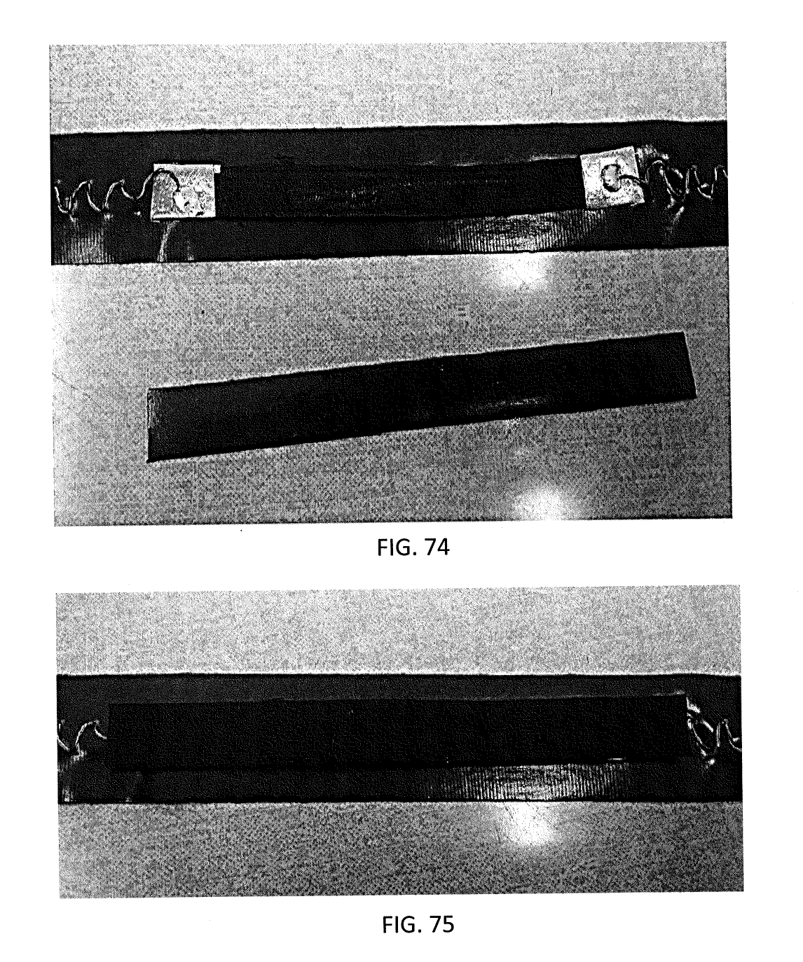

Once applied to the conductive wires, the elastic ribbon may be enclosed within a fabric (e.g., an insulating fabric, which may be the same as the fabric to which it's being applied). In some variations the elastic ribbon may be enclosed in an insulator material and/or coated with an insulator. In FIGS. 74 and 75 the external side of the conductive elastic ribbon (including the contacts) is sealed with an adhesive tissue ribbon to a width of approximately 33 mm). The tissue (covering) ribbon may be fixed over the elastic ribbon by, e.g., thermo press (when using a thermally activated adhesive) as shown in FIG. 75.

Thereafter, the resulting ribbon including the conductive elastic material and zig-zag wires may be attached to a garment, such as a compression garment.

### Example

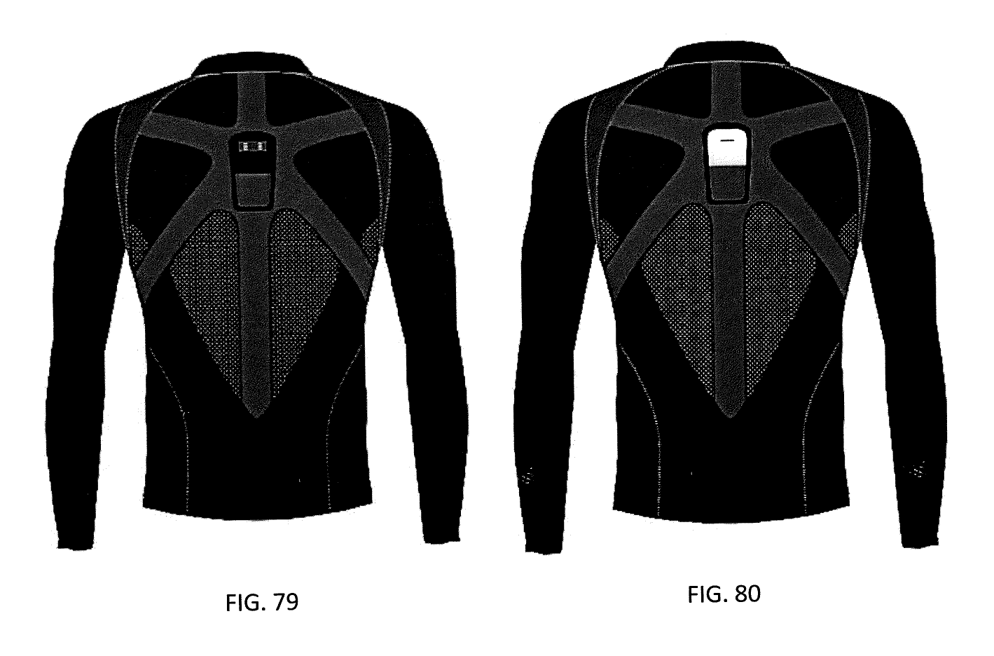

FIGS. 79 and 80 illustrate example of a SMS networks on garments. The data acquisition from the sensors is managed by the SMS network. The SMS network in this example is based on a Cortex M3 MCU, is embedded into a shirt, with the primary SMS module just under the neck (FIG. 79). During usage, Phone Module and SMS stay connected through a mechanical coupling system.

In this example, the phone is also inserted into a pocket that may offer a better support to it (FIG. 80). In addition to this physical connection, the two devices may also share an electrical connection that allows the exchange of data through a serial digital port.

Neither the SMS system, nor the shirt (or tights) are provided with their own source or power supply (e.g. battery) in this example, thus, by using the electrical connection mentioned above, the Phone Module battery may be also used to supply the complete system. The apparatus may be used for sport and outdoors activities, such as running, parkour, cycling, etc. Other applications are related to the medical field, where this kind of system can be used for monitoring patients over long periods of time (e.g. Holter electrocardiogram).

This configuration may prevent failure due close contact of the Phone Module with the human body, by maintaining lightness and small dimensions, limiting the relative movement between Phone Module and SMS caused by the movements of the body during sport activities, etc.

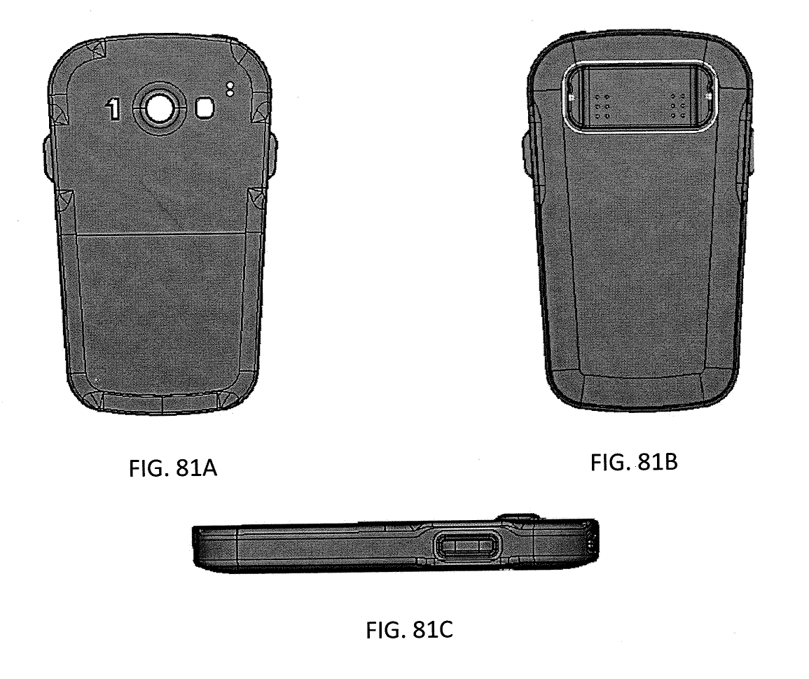

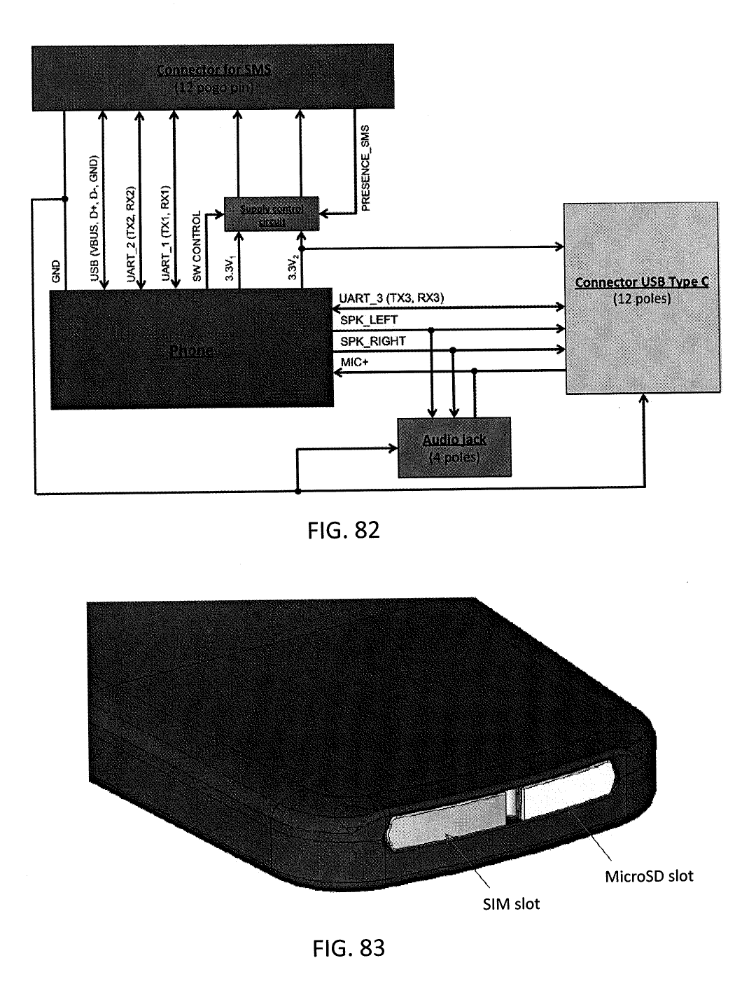

FIGS. 81A-81C illustrate another example of a phone module similar to that discussed above. FIG. 82 shows a schematic of this example. FIG. 83 shows an example of a bottom of a phone, including slots for SIM cards and MicroSD cards.

In this example, the phone has four main I/O ports: one USB port, for data communication and battery charging, two serial UART ports (logic value @+3.3V, FIG. 85), for data communication with the SMS and other external accessory sensors, and another serial UART port (logic value @+3.3V), for data communication with accessories connected to the USB type C connector.

All the UART ports come from the MT6735 CPU, thus their usage was enabled by default at the Android OS level. With the exception of the UART line connected to the USB type C port, all the mentioned I/O ports are connected to the Phone Module pogo pin placed inside the connector for the primary SMS module.

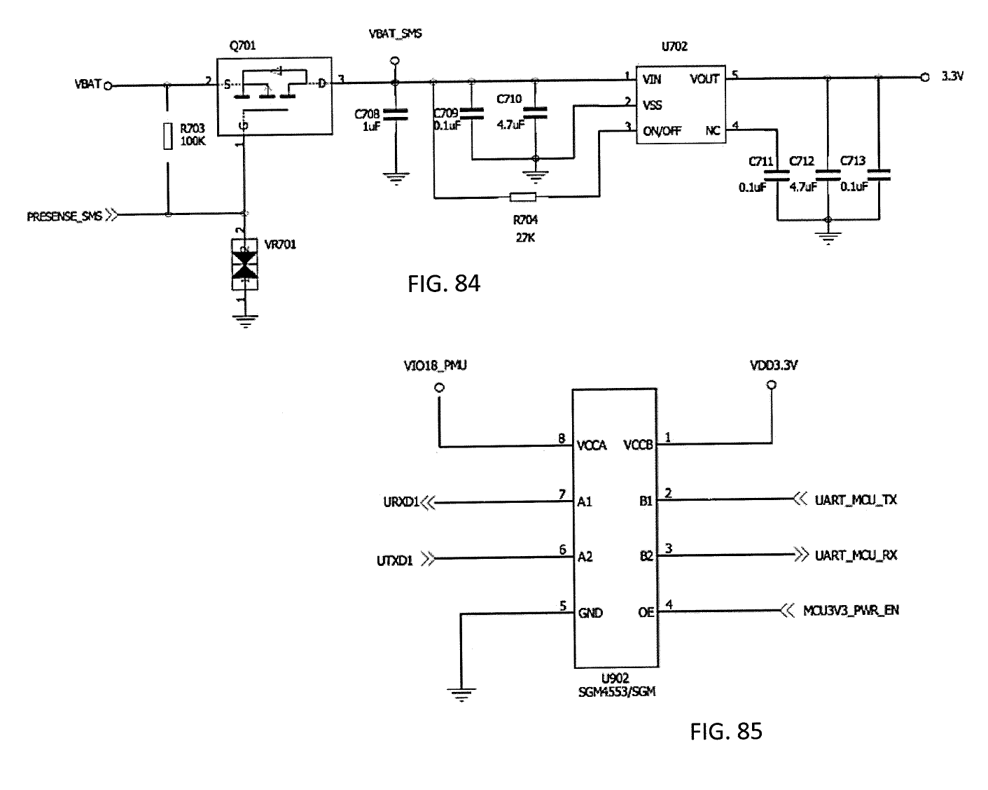

In this example, the phone module has two output supply lines (VOUT=+3.3V and IOUT=250 mA) both connected to two of the twelve pogo pin placed inside the primary SMS connector. These lines are used to supply the SMS network, the sensors (e.g., IMU and MCU embedded into the garment) and other external accessories (e.g. external sensors connected to the garment). It follows that these lines may ensure a stable and reliable source of supply over long period of time, regardless the battery load and charge state. Both the two lines are disabled by default and enabled only when Phone Module and SMS are connected together. A specific control circuit inside the Phone (FIG. 84), based on a P-MOS transistor, is used for this purpose.

Whenever the SMS is connected to the Phone (the Phone is turned ON), PRESENCE_SMS, which is the name of one of the pogo pin, is driven to GND level. This will enable the two +3.3V output supply lines. The LDO U702 must guarantee an output voltage of +3.3V and an average maximum output current of 250 mA (33V1). The same concept is applied to the second supply line (3.3V2).

If the Phone Module is turned off, the two lines do not give any power, not even when the SMS is connected.

In addition to the mentioned hardware control, that system may also implement a software control that will be used to disable/enable the power supply lines, e.g., through the Android OS. This may help management of the firmware updates of the sensors (e.g. supply_line(true); and supply_line(false);).

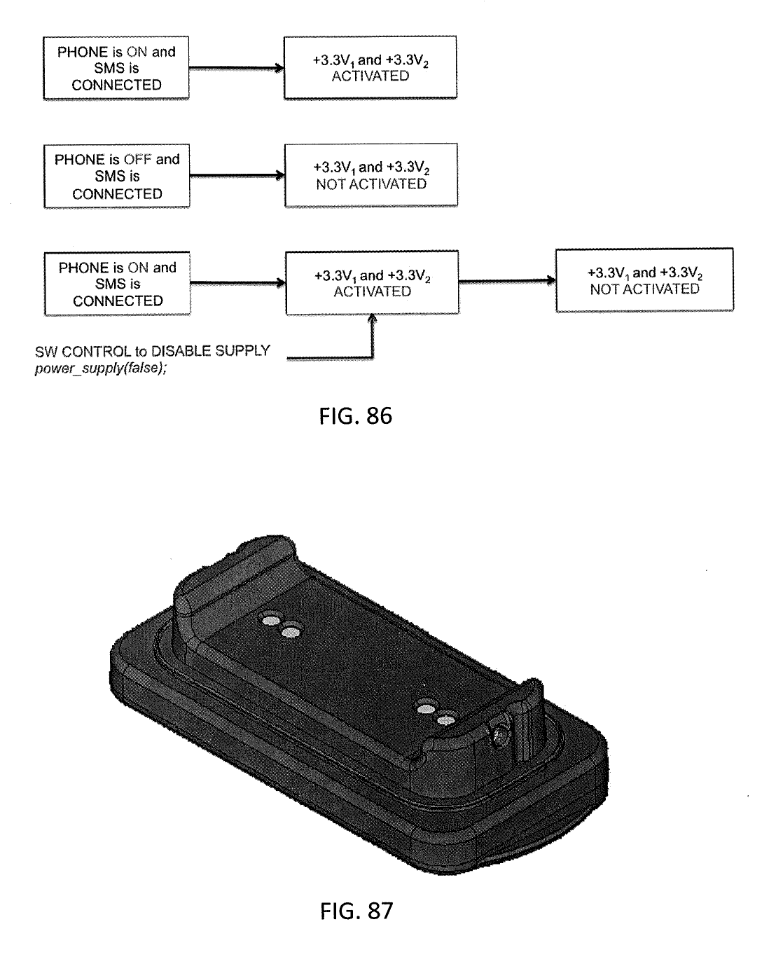

A recap of the way the power supply management may work is shown in FIG. 86. The phone may be charged, e.g., with a charger, such as the one shown in FIG. 87.

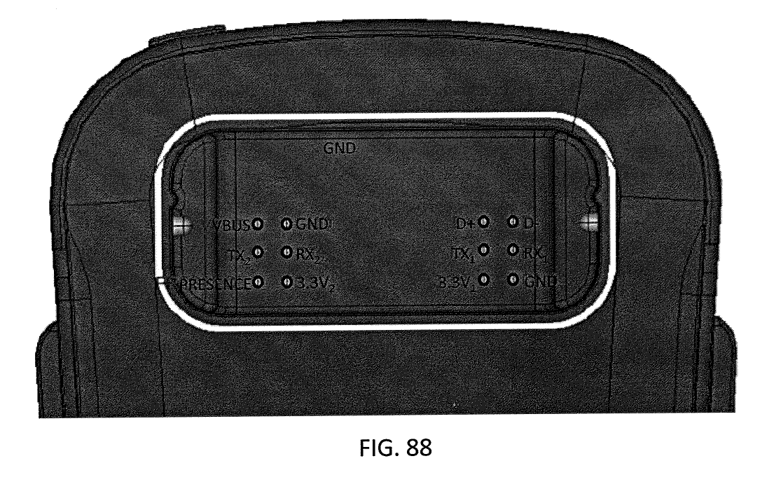

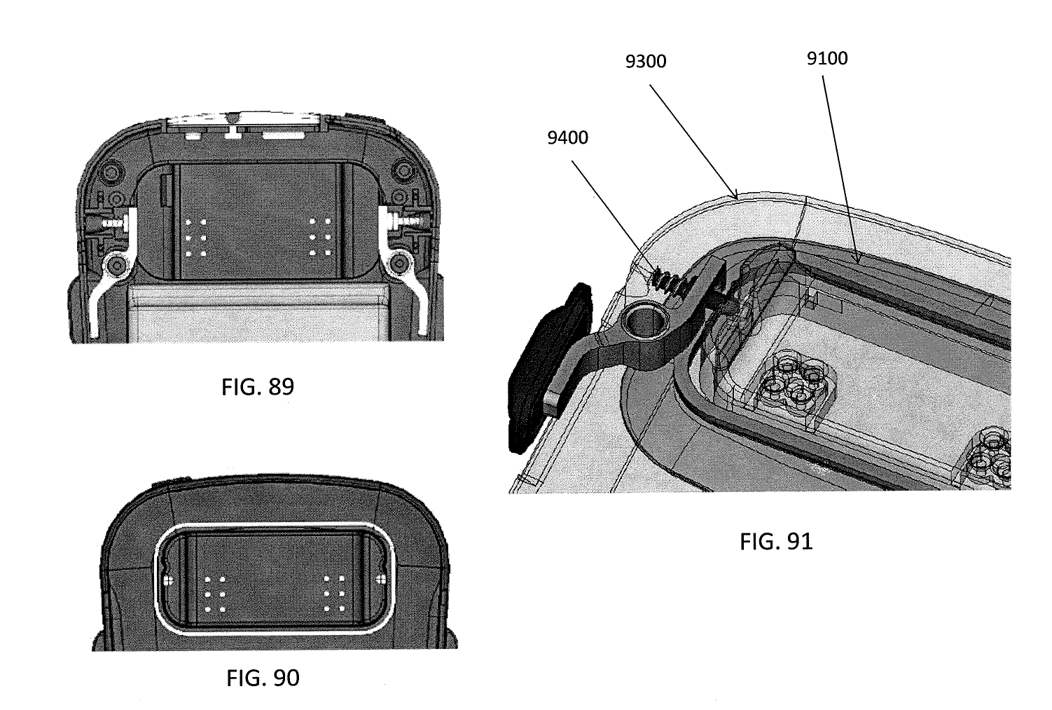

FIG. 88 shows an example of a set of connectors on the back of a phone that may connect to the primary SMS module, as discussed above. FIGS. 89-91 illustrate a mechanical connection between a primary SMS module 9100 and a phone 9300. The coupling mechanism between Phone Module and SMS in this example is ensured by a locking mechanism based on two push buttons placed on the side of the Phone (FIG. 91) and two metallic pins (9400, FIG. 91) able to lock tougher the SMS and Phone Module housings. The SMS can be connected to the Phone Module just by pushing the Phone onto the SMS, while in order to disconnect them it is necessary to push the two rubber buttons placed on the side of the Phone; this will release the lock mechanism (the two pins 9400).

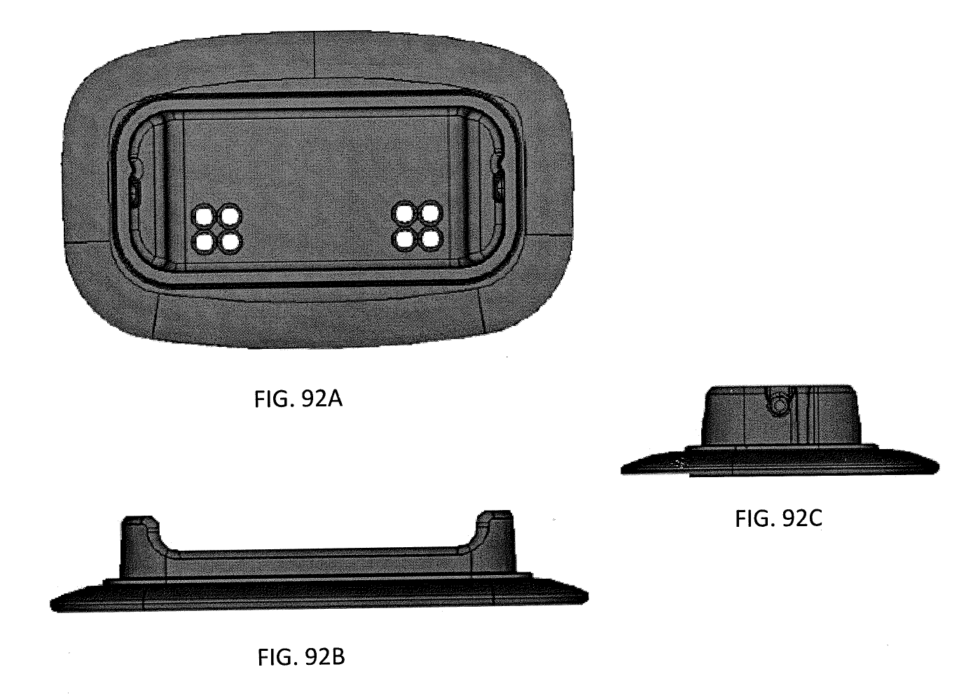

FIGS. 92A-92C show another variation of a primary SMS module.

An SMS module may be embedded into the back of a garment. The primary SMS module may be composed by a single PCB based on, e.g., a Cortex M3 MCU made by Cypress. This PCB module may be embedded into a plastic housing able to protect the electronics. This plastic case may also embed the metal contacts (pins) used to ensure the electrical connection with the Phone Module pogo pins.

The SMS may be part of the garment and may be fully waterproof since garments may be washed by the users after each use. The waterproof capability may be aided by a layer of epoxy resin between the PCB and the pin used for the connection with the Phone Module. A second layer of resin may be placed on the bottom of the SMS PCB, thus covering and protecting the connections with the sensors (these connections are made by wires that come from the sensors that are eventually soldered onto the bottom of the SMS PCB).

Another variation of the SMS may be used with some variation of garments. For example, garments that include sensors for monitoring biological signals (e.g. electrocardiogram ECG and/or electroencephalogram EEG) at medical grade (e.g. 12 lead ECG) may include specific (custom) SMS modules and/or nodes.

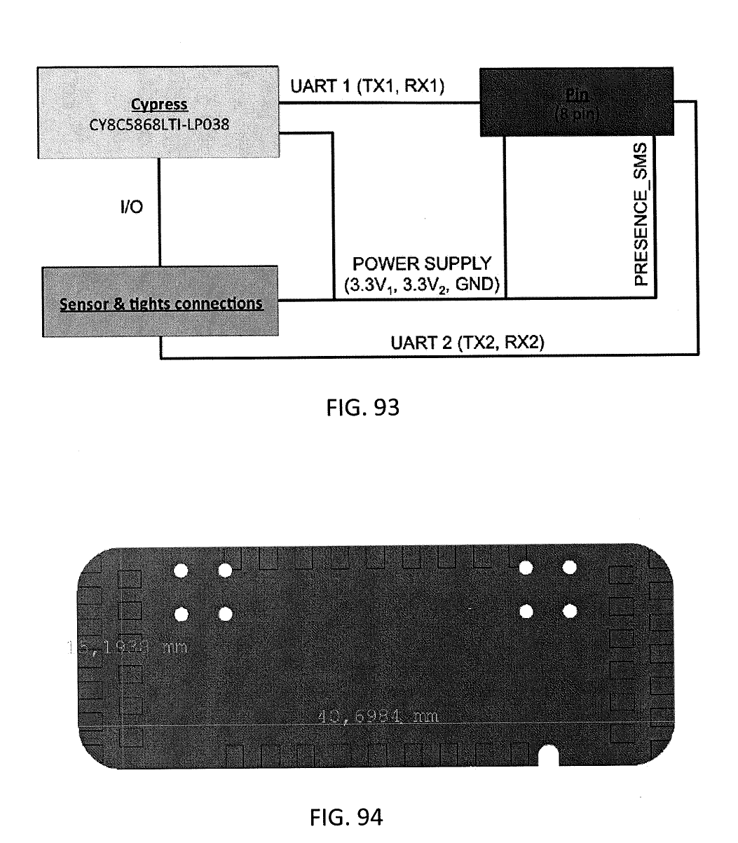

FIG. 93 is a block diagram of an SMS device which underlines the connections between an SMS system on a garment (upper left), a second garment (lower left) and a phone (upper right).

FIG. 94 shows one example of an SMS PCB having dimensions of 40.69×15.19 mm.

The PCB has a rectangular shape with rounded corners. The orientation cut on the PCB is not required since the eight drills/holes for the metal contacts are not symmetrical. The PCBA total thickness may be 4 mm (with 1.0 mm PCB).

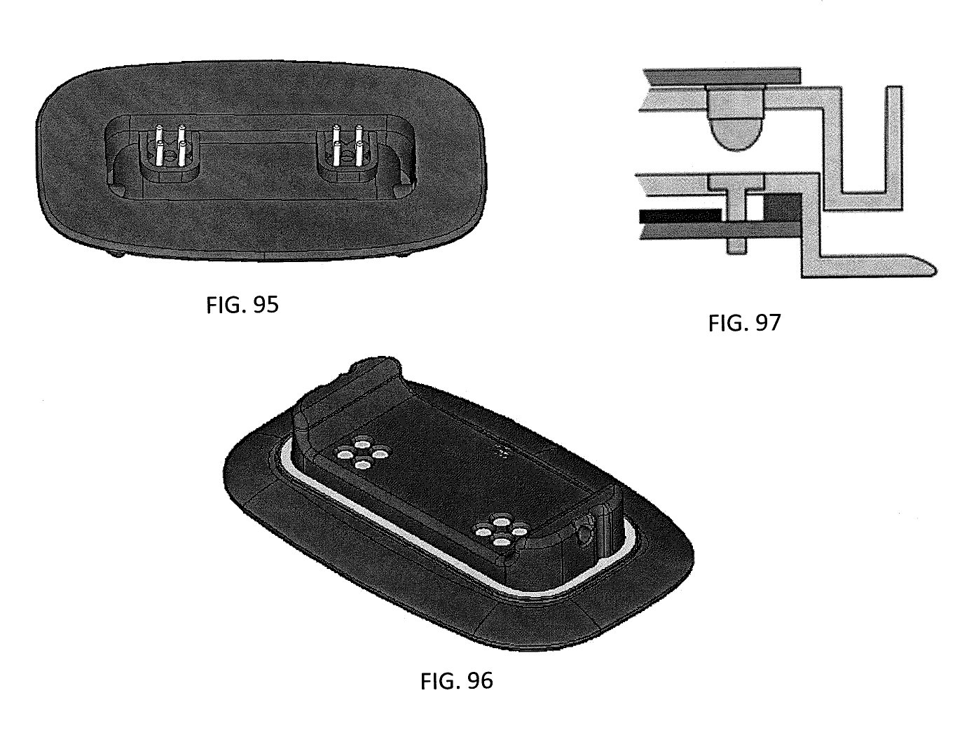

FIGS. 95-97 illustrate one example of a housing for an SMS module (primary SMS module). The SMS housing in this example is made of Polycarbonate or Polycarbonate+ABS and its walls thickness is between 1.5 mm to 2 mm. The SMS housing may be used with an epoxy resin compound poured inside the SMS housing as illustrated above, e.g., between the PCB and the metal contacts. Each group of contacts (on the SMS the eight contacts are dived into two groups of four) is surrounded by walls (height 2 mm) that define the areas where the resin must be poured (FIG. 97). In addition a second layer of resin will be poured on top of the SMS PCB. The walls may be used as a base for placing the SMS PCBA as shown in FIG. 97. The height of 2 mm may allow enough space for the electronic components (mainly the Cypress MCU) placed on the PCB top layer, which faces the bottom of the SMS housing.

As mentioned above, a Phone Module may use twelve pogo pin to ensure an electrical connection with the primary SMS module. The SMS may be provided with eight gold-coated contacts that ensure the electrical connection with the pogo pin. Those contacts may go through the SMS PCB (where there are eight metallized drills) and will be completely surrounded by resin as described into the previous section.

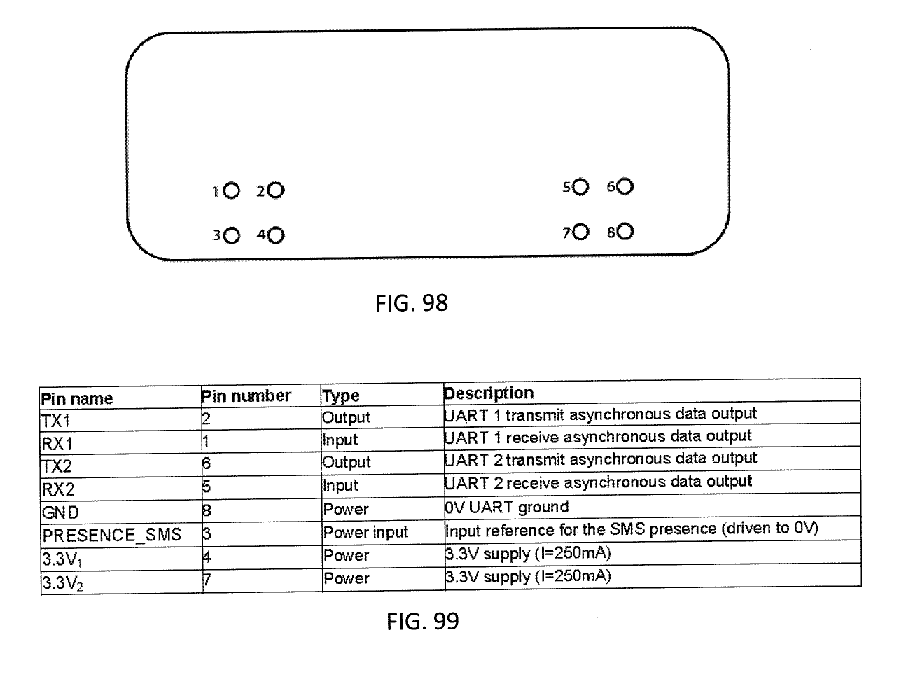

An example of a pinout of the SMS contacts is shown in the table of FIG. 99, with reference to FIG. 98.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A garment, the garment comprising:
a garment body formed of a first fabric;
a plurality of sensors permanently affixed to the garment body;
a primary sensor management system (SMS) module, wherein the primary SMS module includes: a phone securement connector, a plurality of primary sensor data inputs, a plurality of electrical output connectors, and a housing enclosing processing circuitry coupled to the plurality of primary sensor data inputs and the plurality of electrical output connectors;
a plurality of secondary SMS nodes, wherein the secondary SMS nodes each include: a plurality of node sensor inputs receiving input from one or more of the plurality of sensors, one or more node sensor outputs and node processing circuitry comprising a digitizer and an encoder configured to sample analog sensor data from the node sensor inputs and encode a digital representation of the analog sensor data for transmission on the one or more node sensor outputs; and
one or more elastic electrical connectors attached to the garment body and connecting the plurality of secondary SMS nodes to the primary SMS module, wherein the one or more elastic electrical connectors each include: an elongate strip of a second fabric, and a plurality of insulated wires extending along a length of one side of the elongate strip in a sinusoidal or zig-zag pattern;
wherein the primary SMS module is affixed to the garment body on an outer central back portion of the garment body so that the primary SMS module is positioned on a wearer's upper back when the wearer is wearing the garment.

2. The garment of claim 1, wherein the housing of the primary SMS module includes a projection region configured to prevent access to a charging port on a phone attached to the phone securement connector.

3. The garment of any of claims 1-2, wherein the plurality of sensors comprises a plurality of n sensors and the plurality of sensor data inputs comprises m sensor data inputs, where m is less than n.

4. The garment of any of claims 1-3, wherein the garment body comprises one of: a shirt, a bra, and a harness.

5. The garment of any of claims 1-4, wherein the plurality of sensors comprises one or more of: stretch sensors, pressure sensors, capacitive sensors, inductive sensors, electrodes, touch point sensors, accelerometers, inertial measurement (IMU) sensors.

6. The garment of any of claims 1-5, wherein the plurality of sensor comprises ECG electrodes.

7. The garment of any of claims 1-6, wherein the phone securement connector is configured to removably secure a phone to the garment.

8. The garment of any of claims 1-7, wherein the phone securement connector comprises one or more of: a magnetic connector or a mechanical connector.

9. The garment of any of claims 1-8, wherein the plurality of electrical output connectors comprise pin connectors.

10. The garment of any of claims 1-9, wherein the processing circuitry of the primary SMS is configured to serially transfer digitized sensor data received by the primary sensor data inputs to one or more of the electrical output connectors.

11. The garment of any of claims 1-10, further comprising a phone having a mate for the phone securement connector, to releasably secure the phone to the primary SMS sensor module.

12. The garment of any of claims 1-11, wherein the primary sensor management system is integrated on an outer portion of the garment body so that the phone securement connector is exposed.

13. The garment of any of claims 1-12, wherein at least some of the plurality of secondary SMS nodes comprises an epoxy covering the processing circuitry.

14. The garment of any of claims 1-13, wherein each of the plurality of secondary SMS nodes is not covered by a housing.

15. The garment of any of claims 1-14, wherein the processing circuitry of each secondary SMS node is configured to sample received analog sensor data at a first rate and to transmit the digital representation of the analog sensor data at a second data rate that is slower than the first data rate.

16. The garment of any of claims 1-15, wherein one or both of the primary sensor management system and the secondary SMS node comprises a memory for storing sensor data, representations of sensor data, or sensor data and representations of sensor data.

17. The garment of any of claims 1-16, wherein the one or more elastic electrical connectors are adhesively attached to the garment body.

18. The garment of any of claims 1-17, wherein at least some of the plurality of secondary SMS nodes are affixed to the elongate strip of a second fabric of the one or more electrical connectors.

19. The garment of any of claims 1-18, wherein each of the insulated wires is electrically insulated with a thermoremovable insulator.

20. The garment of any of claims 1-19, wherein the insulated wires comprise a bundle of insulated wires that are attached to one side of the elongate strip of second fabric by a stitch at each of a peak and a trough of the sinusoidal or zig-zag pattern, and wherein the length between peak and trough stitches is between about 1 mm and 15 mm.

21. The garment of any of claims 1-20, wherein the first fabric and the second fabric are formed from the same material.

22. The garment of any of claims 1-21, wherein the first fabric comprises a compression fabric.

23. The garment of any of claims 1-22, wherein at least one of the sensors of the plurality of sensors is connected directly to a sensor data input of the primary SMS module.

24. The garment of any of claims 1-23, wherein the primary SMS module housing includes an interface portion, the interface portion including a sensor module interface and a charging interface; wherein the sensor module interface further configured to receive the signals from the plurality of interactive sensors such that the charging interface is fully or partially covered.

25. The garment of claim 24, wherein the charging interface is configured to receive electrical energy from an electrical energy source configured to engage with the interface portion and charging interface such that the sensor module interface is partially or fully covered.

26. The garment of claim 24, wherein the interface portion, charging interface, and sensor module interface are configured such that the sensor module interface and charging interface cannot be used simultaneously.

27. The garment of any of claims 1-24, wherein the primary SMS module comprises a battery.

28. The garment of any of claims 1-24, wherein the one or more elastic electrical connectors has a maximum thickness of less than about 2 mm.

29. The garment of any of claims 1-24, wherein the sinusoid or zig-zag pattern has an amplitude from about 0.5 mm to 15 mm.

30. The garment of any of claims 1-24, wherein the one or more elastic electrical connectors has an adhesive coating comprising a hot melt film having a low melting point.

31. The garment of any of claims 1-24, wherein the one or more elastic electrical connectors has an adhesive coating having a thickness of between 10 and 200 micrometers thick.

32. The garment of any of claims 1-24, wherein the plurality of insulated wires comprises between 2 and 10 wires.

33. The garment of any of claims 1-24, wherein either or both the first and second fabric comprises a polyamide/elastane blend fabric.

34. A garment, the garment comprising:
a garment body formed of a first fabric;
a plurality of n sensors permanently affixed to the garment body;
a primary sensor management system (SMS) module, wherein the primary SMS module includes: a phone securement connector, a plurality of m primary sensor data inputs, where m is less than n, a plurality of electrical output connectors, and processing circuitry coupled to the plurality of primary sensor data inputs and digitally encoding sensor output for transmission from the plurality of electrical output connectors;
a plurality of secondary SMS nodes, wherein the secondary SMS nodes each include: a plurality of node sensor inputs receiving input from one or more of the plurality of sensors, one or more node sensor outputs and node processing circuitry comprising a digitizer and an encoder configured to sample analog sensor data from the node sensor inputs and encode a digital representation of the analog sensor data for transmission on the one or more node sensor outputs; and
one or more electrical connectors connecting the plurality of secondary SMS nodes to the primary SMS module, wherein the one or more elastic electrical connectors each include: a plurality of insulated wires extending in a sinusoidal or zig-zag pattern;
wherein the primary SMS module is affixed to the garment body on an outer central back portion of the garment body so that the primary SMS module is positioned on a wearer's upper back when the wearer is wearing the garment.

35. A garment, the garment comprising:
a garment body formed of a first fabric;
a plurality of n sensors permanently affixed to the garment body;
a primary sensor management system (SMS) module, wherein the primary SMS module includes: a phone securement connector, a plurality of m primary sensor data inputs, where m is less than n, a plurality of electrical output connectors, and processing circuitry coupled to the plurality of primary sensor data inputs and the plurality of electrical output connectors;
a plurality of secondary SMS nodes, wherein the secondary SMS nodes each include: a plurality of node sensor inputs receiving input from one or more of the plurality of sensors, one or more node sensor outputs and node processing circuitry connected to the plurality of node sensor inputs and the one or more node sensor outputs; and
one or more electrical connectors connecting the plurality of secondary SMS nodes to the primary SMS module.
